(19) United States
(12) Patent Application Publication
(10) Pub. No.: US 2009/0233955 A1
(43) Pub. Date: Sep. 17, 2009

(54) 1H-Pyrrolo[2,3-B]Pyridnes

(76) Inventors: James S. Frazee, King of Prussia, PA (US); Marlys Hammond, King of Prussia, PA (US); Sharada Manns, King of Prussia, PA (US); Scott Kevin Thompson, King of Prussia, PA (US); David G. Washburn, King of Prussia, PA (US); Kazuya Kano, Ibaraki (JP); Hiroko Nakamura, Ibaraki (JP)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/720,951

(22) PCT Filed: Dec. 8, 2005
(86) PCT No.: PCT/US05/44485

§ 371 Date: May 21, 2009

Related U.S. Application Data
(60) Provisional application No. 60/634,149, filed on Dec. 8, 2004.

(51) Int. Cl. A61K 31/437; C07D 471/04; A61P 9/00

(52) U.S. Cl. 514/300; 546/113

(57) ABSTRACT

Derivatives of pyrrolo[2,3-b]pyridine which are useful as SGK-1 kinase inhibitors are described herein. The invention described herein also describes pharmaceutical compositions containing derivatives of pyrrolo[2,3-b]pyridine and methods of using pyrrolo[2,3-b]pyridine derivatives and pharmaceutical compositions thereof in the treatment of diseases mediated by SGK-1.

### FIELD OF THE INVENTION

This invention relates to novel compounds, compositions containing them, their use as inhibitors of SGK-1 kinase, and their use in the treatment of diseases mediated at least in part by SGK-1 kinase.

### BACKGROUND OF THE INVENTION

An important large family of enzymes is the protein kinase enzyme family. Currently, there are about 400 different known protein kinases. However, because three to four percent of the human genome is a code for the formation of protein kinases, there may be many thousands of distinct and separate kinases in the human body. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

Serum and Glucocorticoid-Regulated Kinase 1 (SGK-1) is a serine/threonine protein kinase, whose function is thought linked to cell proliferation and electrolyte homeostasis. SGK-1 is a member of a family of intracellular kinases which includes protein kinase B. While it is transcriptionally induced by glucocorticoids and mineralocorticoids, it is activated by insulin and IGF-1 mediated phosphorylation through PI3-kinase and PDK-1. SGK-1 is thought to mediate several mechanisms, which contribute to disease states. As indicated above, IGF-1 activates SGK-1 and is involved in fibronectin synthesis, an element of renal fibrosis. Consequently, SGK-1 may mediate IGF-1 action on fibronectin synthesis. The anti-diuretic aldosterone induces expression of SGK-1, which in turn activates the epithelial Na+ channel thereby affecting Na+ transport. Accordingly, SGK-1 may serve to mediate aldosterone-induced Na+ retention in renal and cardiovascular disease. SGK-1 may also mediate repair processes involving cell proliferation, for instance, through thrombin. Thrombin causes renal cell proliferation and increases SGK-1 expression in renal cells. Therefore, SGK-1 may provide a novel therapy for the regulation of electrolyte balance in renal and cardiovascular disease and in damaging cell proliferation in renal disease.

The present inventors have discovered novel 1H-pyrrolo[2,3-b]pyridine compounds and/or methods for inhibition of SGK-1 kinase activity. The 1H-pyrrolo[2,3-b]pyridines described herein are useful in the treatment of disorders associated with SGK-1 activity.

### SUMMARY OF THE INVENTION

This invention comprises methods of treatment of disorders in mammals which are mediated at least in part by SGK-1 kinase through administration of a compound of formula (I) or a pharmaceutically acceptable salt, solvate or pharmaceutical composition thereof. This invention also comprises compounds of formula (II) and pharmaceutically acceptable salts, solvates or pharmaceutical compositions thereof, wherein the compounds of formula (II) are useful in the treatment of disorders which are mediated at least in part by SGK-1 kinase. This invention also comprises compounds of formula III and fluorescent kinase ligands of formula IV.

### DETAILED DESCRIPTION OF THE INVENTION

In one embodiment, this invention provides a method of treating a disorder in a mammal, said disorder being mediated by SGK activity, comprising administering to said mammal a therapeutically effective amount of a compound of formula (I),

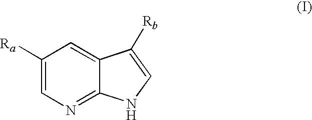

wherein:

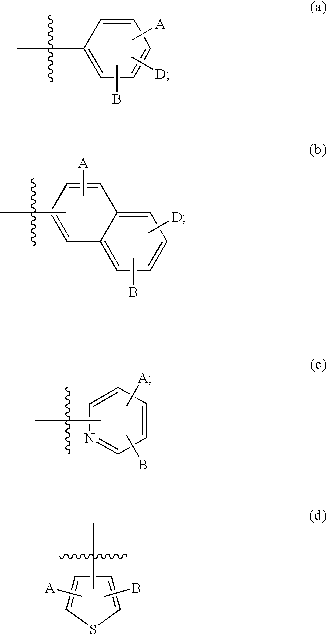

### Rₐ is

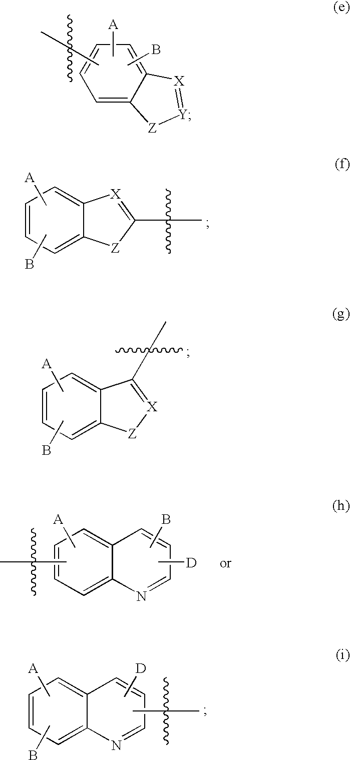

wherein:

A, B, D, and R^{1a} are each independently hydrogen; OR; CN; halogen; CO₂R;

CONR₁R₂; NR₁R₂; NR₃R₄; aryl; heteroaryl; (C₁₋₃)alkyl-NR₁R₂; (C₁₋₆)alkyl, or (C₁₋₆)haloalkyl;

X and Y are each independently CR^{1a} or N;

Z is NR, O or S;

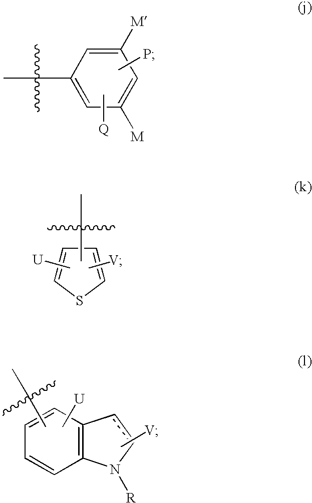

### R_{b} is

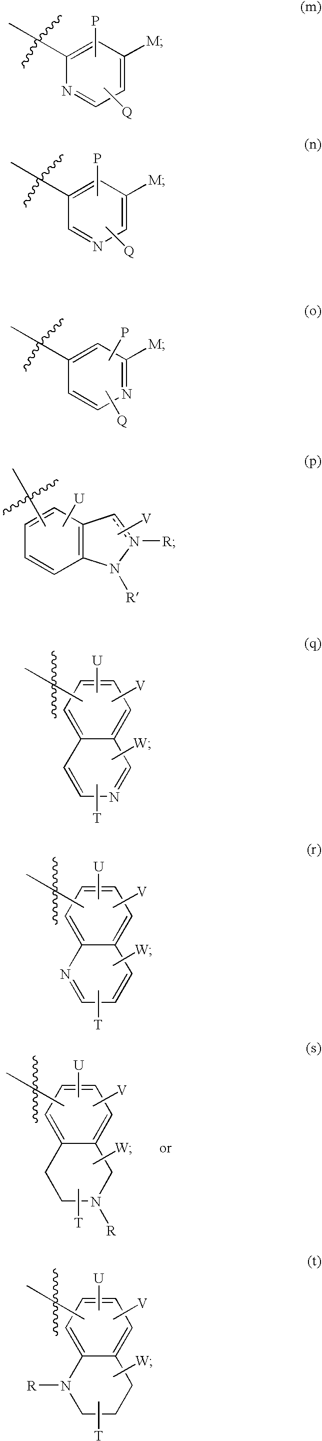

M is independently hydrogen; (C₁₋₃)alkyl-NR₁R₂; (C₁₋₃)alkyl-OR; halogen; CO₂R; OR; NR₁R₂; (C₁₋₃)alkyl-NR₃R₄; CONR₁R₂; (C₁₋₆)alkyl-CONR₁R₂; CHO; (C₁₋₆)alkylCO₂R; (C₁₋₆)alkyl; or NR₃R₄;

M′ is independently hydrogen; (C₁₋₃)alkyl-NR₁R₂; (C₁₋₃)alkyl-OR; halogen; CO₂R; OR; NR₁R₂; (C₁₋₃)alkyl-NR₃R₄; CONR₁R₂; (C₁₋₆)alkyl-CONR₁R₂; CHO; (C₁₋₆)alkylCO₂R; NR₃R₄; (C₁₋₆)alkyl; or phenyl;

P, Q, T, U, V and W are each independently hydrogen; halogen; (C₁₋₆)alkyl; (C₁₋₃)alkylOR; (C₁₋₆)haloalkyl; CO₂R; CHO; (C₁₋₆)alkyl-CO₂R; (C₁₋₆)alkyl-NR₁R₂; OR; NR₁R₂; NR₃R₄; CONR₁R₂; (C₁₋₆)alkyl-CONR₁R₂; aryl; or heteroaryl;

R and R′ are independently at each occurrence hydrogen; (C₁₋₃)alkylaryl; (C₁₋₃)alkylheteroaryl; (C₁₋₃)alkyl; or (C₁₋₆)haloalkyl;

R₁ and R₂ are independently at each occurrence hydrogen; (C₁₋₆)alkyl; (C₁₋₃)alkylNRR′; (C₁₋₃)alkylOR; (C₁₋₆)cyanoalkyl; NRR′; (C₁₋₃)alkylaryl, (C₁₋₃)alkylheteroaryl; (C₁₋₆)haloalkyl; or together with the nitrogen that they are attached form a 4, 5, 6, or 7 member non-aromatic ring, said ring optionally containing up to 2 additional heteroatoms selected from NR; 0; or S(O)ₙ; and said ring optionally substituted with from 1-3 substituents selected from halogen; (C₁₋₆)alkyl; OR; NRR′; CN; halogen; (C₁₋₆)haloalkyl; phenyl; heteroaryl or heterocyclyl;

n is independently at each occurrence 0, 1 or 2;

R₃ is independently at each occurrence hydrogen; (C₁₋₆)alkyl; or (C₁₋₆)haloalkyl;

R₄ is C(═O)(C₁₋₆)alkyl; C(═O)(C₁₋₃)alkyl-NRR′; C(═O)—(C₁₋₃)alkylaryl (wherein said aryl is optionally substituted with 1-3 substituents selected from halogen, (C₁₋₃)alkyl and (C₁₋₃)alkoxy); C(═O)—(C₁₋₄)alkylheteroaryl; C(═O)-phenyl (wherein the phenyl group is optionally substituted with 1-3 substituents selected from halogen, (C₁₋₆)alkyl or OR);

or a pharmaceutically acceptable salt or solvate thereof.

In a second embodiment, this invention provides a method of treating a disorder in a mammal, wherein said disorder is a proliferative response to an insult or injury comprising administering to said mammal a therapeutically effective amount of a compound of formula (I).

In a third embodiment, this invention provides a method of treating a disorder in a mammal, wherein said disorder is excess water retention comprising administering to said mammal a therapeutically effective amount of a compound of formula (I).

In a fourth embodiment, this invention provides a method of treating a disorder in a mammal, wherein said disorder is a renal disorder and said method comprises administering to said mammal a therapeutically effective amount of a compound of formula (I).

In a fifth embodiment, this invention provides a method of treating a disorder in a mammal, wherein said disorder is a cardiovascular disease and said method comprises administering to said mammal a therapeutically effective amount of a compound of formula (I).

In a sixth embodiment, this invention describes a compound of formula (II)

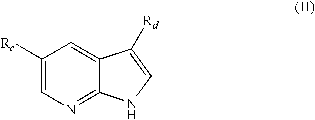

wherein:

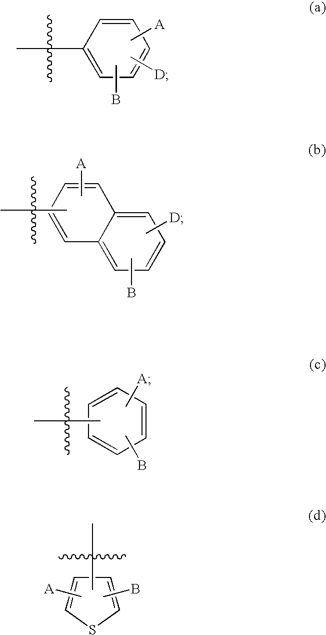

### R_{c} is

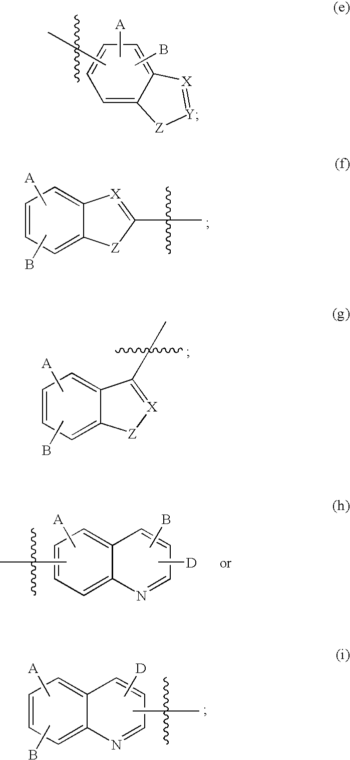

wherein:

A, B, D, and R^{1a} are each independently hydrogen; OR; CN; halogen; CO₂R; CONR₁R₂; NR₁R₂; NR₃R₄; S(O)ₙ(C₁₋₆)alkyl (wherein said alkyl is optionally substituted with 1-3 substituents selected from halogen; OR; NRR′; and CN); aryl; heteroaryl; (C₁₋₃)alkyl-NR₁R₂; (C₁₋₆)alkyl, or (C₁₋₆)haloalkyl;

X and Y are each independently CR^{1a} or N;

Z is NR, O or S;

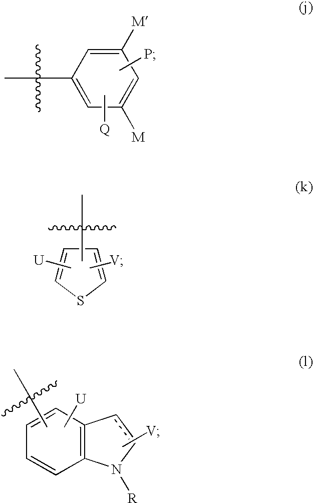

### R^{d} is

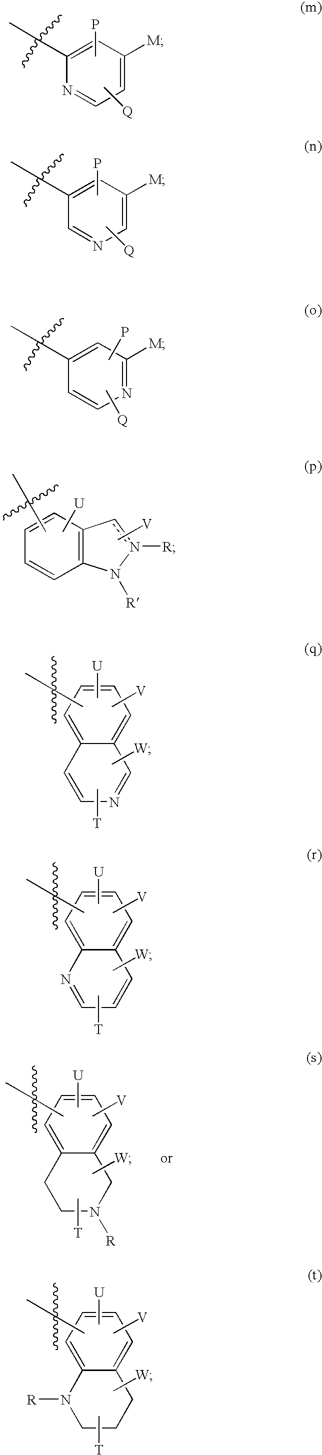

M is independently hydrogen; (C₁₋₂)alkyl-NR₁R₂; (C₁₋₃)alkyl-OR; halogen; CO₂R; OR; S(O)ₙ(C₁₋₆)alkyl (wherein said alkyl is optionally substituted with 1-3 substituents selected from halogen; OR; NRR′; and CN); NR₁R₂; (C₁₋₃)alkyl-NR₃R₄; SO₂NR₁R₂; CONR₁R₂; (C₁₋₆)alkyl-CONR₁R₂; CHO; (CO₁)alkylCO₂R; (C₁₋₆)alkyl; or NR₃R₄;

M′ is independently hydrogen; (C₁₋₃)alkyl-NR₁R₂; (C₁₋₃)alkyl-OR; halogen; CO₂R; OR; S(O)ₙ(C₁₋₆)alkyl (wherein said alkyl is optionally substituted with 1-3 substituents selected from halogen; OR; NRR′; and CN); NR₁R₂; (C₁₋₃)alkyl-NR₃R₄; SO₂NR₁R₂; CONR₁R₂; (CO)alkyl-CONR₁R₂; CHO; (C₁₋₆)alkylCO₂R; NR₃R₄; (C₁₋)alkyl; or phenyl;

P and Q are each independently hydrogen; halogen; (C₁₋₆)alkyl; (C₁₋₃)alkylOR; (C₁₋₆)haloalkyl; CO₂R; CHO; S(O)ₙ(C₁₋₆)alkyl (wherein said alkyl is optionally substituted with 1-3 substituents selected from halogen; OR; NRR′; and CN); (C₁₋₆)alkyl-CO₂R; (C₁₋₃)alkyl-NR₁R₂; (C₁₋₃)alkyl-NR₃R₄; OR; NR₁R₂; NR₃R₄; CONR₁R₂; (C₁₋₆)alkyl-CONR₁R₂; SO₂NR₁R₂; aryl; or heteroaryl;

T, U, V and W are each independently hydrogen; halogen; (C₁₋₆)alkyl; (C₁₋₃)alkylOR; (C₁₋₆)haloalkyl; CO₂R; CHO; S(O)ₙ(C₁₋₆)alkyl (wherein said alkyl is optionally substituted with 1-3 substituents selected from halogen; OR; NRR′; and CN); (C₁₋₆)alkyl-CO₂R; (C₁₋₃)alkyl-NR₁R₂; (C₁₋₃)alkyl-NR₃R₄; OR; NR₁R₂; NR₃R₄; CONR₁R₂; (C₁₋₆)alkyl-CONR₁R₂; SO₂NR₁R₂; aryl; or heteroaryl;

R and R′ are independently at each occurrence hydrogen; (C₁₋₆)alkylaryl; (C₁₋₃)alkylheteroaryl; (C₁₋₆)alkyl; or (C₁₋₆)haloalkyl;

R₁ and R₂ are independently at each occurrence hydrogen; (C₁₋₆)alkyl; (C₁₋₃)alkylNRR′; (C₁₋₃)alkylOR; (C₁₋₆)cyanoalkyl; NRR′; (C₁₋₃)alkylaryl, (C₁₋₃)alkylheteroaryl; (C₁₋₆)haloalkyl; or together with the nitrogen that they are attached form a 4, 5, 6, or 7 member non-aromatic ring, said ring optionally containing up to 2 additional heteroatoms selected from NR; O; or S(O)ₙ; and said ring optionally substituted with from 1-3 substituents selected from halogen; (C₁₋₆)alkyl; OR; NRR′; CN; halogen; (C₁₋₆)haloalkyl; phenyl; heteroaryl or heterocyclyl;

n is independently at each occurrence 0, 1 or 2;

R₃ is independently at each occurrence hydrogen; (C₁₋₆)alkyl; or (C₁₋₆)haloalkyl;

R₄ is C(═O)(C₁₋₆)alkyl; C(═O)(C₁₋₃)alkyl-NRR′; C(═O)—(C₁₋₆)alkylaryl (wherein said aryl is optionally substituted with 1-3 substituents selected from halogen, (C₁₋₃)alkyl and (C₁₋₃)alkoxy); C(═O)—(C₁₋₃)alkylheteroaryl; S(O)ₙ(C₁₋₆)alkyl; SO₂NR₁R₂; C(═O)-phenyl (wherein the phenyl group is optionally substituted with 1-3 substituents selected from halogen, (C₁₋₆)alkyl or OR);

or a pharmaceutically acceptable salt or solvate thereof; and

provided at least one of M, P, and Q is (C₁₋₆)alkyl-CO₂R or (C₁₋₆)alkyl-CONR₁R₂; and

provided at least one of U and V, or at least one of U, V, T and W is (C₁₋₆)alkyl-CO₂R or (C₁₋₆)alkyl-CONR₁R₂

In a seventh embodiment, this invention describes a compound of formula (II) wherein R_{c} is (a), (b), (c) or (d).

In an eighth embodiment, this invention describes a compound according to formula (II) wherein R_{c} is (a).

In a ninth embodiment, this invention describes a compound according to formula (II) wherein R_{d} is (j), (l), (m), (n) or (o).

In a tenth embodiment, this invention describes a compound according to formula (II) wherein R_{d} is (j) and R_{c} is (a), (b), (c) or (d).

In an eleventh embodiment, this invention describes a compound according to formula (II) wherein R_{d} is (j), R_{c} is (a), (b), (c) or (d), and at least one of m, p, or q is (C₁₋₆)alkylCO₂R.

In a twelfth embodiment, this invention describes a compound according to formula (II) wherein R_{d} is (j), R_{c} is (a), (b), (c) or (d), and at least one of m, p, or q is (C₁₋₆)alkylCO₂H.

In a thirteenth embodiment, this invention describes a compound according to formula (I) wherein R_{d} is (j) and at least one of m, p, or q is (C₁₋₆)alkylCO₂R and R_{c} is (a) or (b).

In a fourteenth embodiment, this invention describes a compound according to formula (II) wherein R_{d} is (j) and at least one of m, p, or q is (C₁₋₆)alkylCO₂H and R_{c} is (a) or (b).

In a fifteenth embodiment, this invention relates to and covers one or more of the specific compounds set out in the Preparations and Examples below, or a pharmaceutically acceptable salt or solvate of those compounds; and methods for making same.

In a sixteenth embodiment, this invention describes a pharmaceutical composition comprising a therapeutically effective amount of a compound of formula (II) or any one of the structural embodiments recited herein, or a pharmaceutically acceptable salt or solvate thereof, and one or more of pharmaceutically acceptable carriers, diluents and excipients.

In a seventeenth embodiment, this invention describes a kinase-inhibiting compound of formula (III);

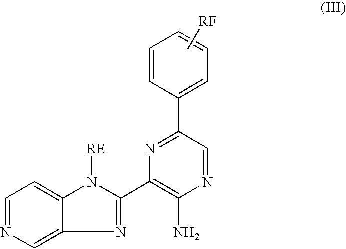

wherein Re is hydrogen, or (C₁₋₆)alkyl; and

Rf is NH(C═O)—(C₁₋₆)alkyl-NH₂, or (CONH)—(C₂₋₄)alkyl-NH₂; or

a pharmaceutically acceptable salt or solvate thereof.

In an eighteenth embodiment, the compound of formula (III) is:

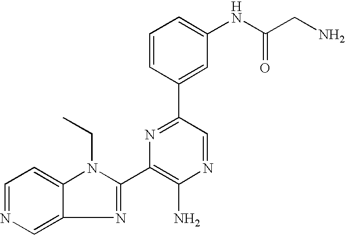

or a pharmaceutically acceptable salt or solvate thereof.

In a nineteenth embodiment, this invention describes a compound of formula (IV), useful as a displaceable ligand in a kinase fluorescent polarization assay

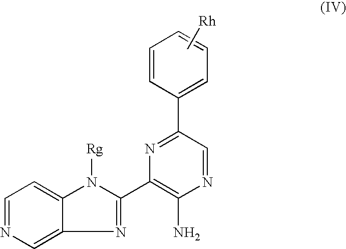

wherein Rg is hydrogen or (C₁₋₆)alkyl, Rh is NH(C═O)—(C₁₋₃)alkyl-NH—Ri, or (CONH)—(C₂₋₄)alkyl-NH—Ri; and Ri is (C═O)—Fl; and Fl is a fluorescent molecule.

In a twentieth embodiment, the compound of formula (IV) is

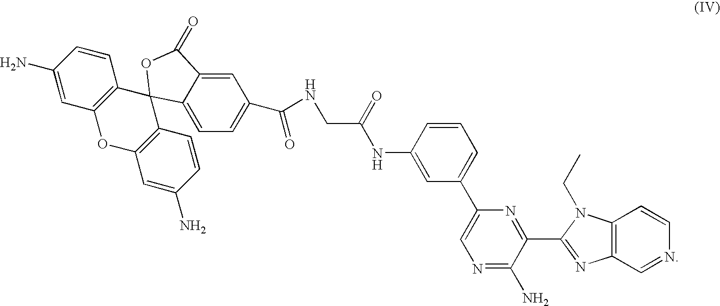

In a twenty-first embodiment, this invention describes a method of measuring a molecule's kinase binding activity comprising the displacement of a fluorescent ligand of formula IV from a kinase enzyme and quantitation of the result.

As described herein, the term “therapeutically effective amount” means that amount of compound or pharmaceutical composition containing said compound that will elicit the particular pharmacological response being sought in the relevant system. Further, the term “therapeutically effective amount” means any amount which results in improved treatment, healing, prevention, effect, or amelioration of a disease, disorder, side effect or condition, or a decrease in the rate of advancement of a disease, disorder or condition. The term also includes within its scope amounts effective to enhance normal physiological function.

As described herein, the term “alkyl” refers to a straight- or branched-chain hydrocarbon radical having no less than one carbon atom and no more than 12 carbon atoms unless specified otherwise. For example, the term “(C₁₋₆) alkyl” refers to an alkyl group containing at least one carbon atom and no more than six carbon atoms. Some non-limiting examples of (C₁₋₆)alkyl radicals include methyl, ethyl, n-propyl, isopropyl, n-butyl, isobutyl, t-butyl, n-pentyl, isopentyl, n-hexyl, and 2-methyl pentane.

As described herein, alkyl groups may be substituted as specified. The term “haloalkyl” refers to an alkyl group as otherwise defined that is further substituted with one or more halogen groups. By way of non-limiting example, the term “C₁₋₆ haloalkyl” includes such radicals as trifluoromethyl, 2-chloroethyl, pentafluoroethyl, [2,2,2-trifluoro-1-(trifluoromethyl)ethyl], and the like. Additional examples encountered herein include such descriptors as (C₁₋₆)alkylCO₂R. Thus, the aforementioned (C₁₋₆)alkylCO₂R will be understood to include such substituents as —C(CH₃)₂—CO₂R, —CH(CH₂CH₃)CH₂—CO₂R, and the like.

In some cases, an alkyl group as defined herein may be present as a diradical, for example, the group O—(C₁₋₄)alkyl-phenyl. In such cases, the alkyl group may be straight chain or branched as previously explained.

As described herein, the term “alkylaryl” refers to an alkyl radical which is substituted with an aryl group, where the term “alkyl” of the specified chain length and “aryl” are as defined herein. By way of non-limiting example, the term (C₁₋₃)alkylaryl includes such radicals as benzyl, 1-phenylethyl, 2-phenylethyl, 1-naphthylethyl, and the like.

As described herein, the term “alkylheteroaryl” refers to an alkyl radical of the specified chain length which is substituted at some point with a heteroaryl group, where the term “heteroaryl” and “alkyl” are as otherwise defined herein. By way of non-limiting example, the term “(C₁₋₃)alkylheteroaryl” includes such radicals as 2-(2-pyridylethyl), 3-(2-pyridylethyl), 1-(3-propyl-1H-pyrrole), and the like.

As described herein, the term “aryl” refers to an optionally substituted benzene ring or to an optionally substituted benzene ring fused to an additional ring, wherein said additional ring may be a benzene ring, a dihydrobenzene ring, a tetrahydro benzene ring, a cyclopentene ring, a cyclopentane ring, a cycloheptadiene ring, a cycloheptene ring, or a cycloheptane ring. The aryl radical may be connected anywhere synthetically accessible and unless otherwise specified, the aryl group is optionally substituted with from 1- to 5-groups selected from halogen, (C₁₋₆)alkyl, (C₁₋₆)alkoxy, (C₁₋₆)haloalkyl, oxo, hydroxyl, ((C₁₋₆)alkyl)₂N, CO₂(C₁₋₆)alkyl, CHO, CO₂H, S—(C₁₋₆)alkyl, CON((C₁₋₆)alkyl)₂; (C₁₋₆)alkylCO₂H, (C₁₋₆)alkylCO₂(C₁₋₆)alkyl, C(═O)—(C₁₋₆)alkyl, C(═O)-phenyl (wherein said phenyl is optionally substituted with from 1- to 3-groups selected from halogen, (C₁₋₃)alkyl or (C₁₋₃)alkoxy), O—(C₁₋₄)alkyl-N((C₁₋₆)alkyl)₂; O—(C₁₋₄)alkyl-heterocyclyl (wherein said heterocyclyl is optionally substituted with from 1- to 3-groups selected from halogen, (C₁₋₃)alkyl or (C₁₋₃)alkoxy), O—(C₁₋₄)alkyl-heteroaryl (wherein said heteroaryl is optionally substituted with from 1- to 3-groups selected from halogen, (C₁₋₃)alkyl or (C₁₋₃)alkoxy), O—(C₁₋₄)alkyl-phenyl (wherein said phenyl is optionally substituted with from 1- to 3-groups selected from halogen, (C₁₋₃)alkyl or (C₁₋₃)alkoxy) and (C₁₋₆)alkylN((C₁₋₆)alkyl)₂. Some non-limiting examples of “aryl” as used herein include benzene, naphthalene, 1,2,3,4-tetrahydronaphthalene, benzocycloheptane and substituted versions thereof.

As described herein, the term “heteroaryl” refers to a monocyclic, bicyclic or tricyclic ring system having a total of from five- to seventeen-backbone atoms, wherein at least one of the rings is aromatic, and each ring contains from five- to seven-backbone atoms. The “heteroaryl” ring contains from one- to four-heteroatoms in the backbone independently selected from N, O and S. When a backbone sulfur or sulfurs are present, each sulfur may be independently present as S, SO or SO₂. When a nitrogen atom or nitrogen atoms are present in the backbone, each nitrogen atom is optionally and independently substituted with (C₁₋₆)alkyl, (C₁₋₃)alkylaryl (wherein said aryl is phenyl and is optionally substituted with from 1- to 3-groups selected from halogen, (C₁₋₃)alkyl and (C₁₋₃)alkoxy), C(═O)—(C₁₋₆)alkyl, CO₂—(C₁₋₆)alkyl, CO₂—(C₁₋₃)alkylaryl (wherein said aryl is phenyl and said phenyl is optionally substituted with 1- to 3-substituents independently selected from halogen, (C₁₋₃)alkyl and (C₁₋₃)alkoxy), (C═O)-phenyl (wherein said phenyl is optionally substituted with 1- to 3-substituents selected from halogen, (C₁₋₃)alkyl and (C₁₋₃)alkoxy). Unless otherwise specified, the “heteroaryl” ring may be further substituted with 1- to 5-groups selected from halogen, (C₁₋₆)alkyl, (C₁₋₆)alkoxy, (C₁₋₆)haloalkyl, oxo, hydroxyl, ((C₁₋₆)alkyl)₂N, CO₂(C₁₋₆)alkyl, CHO, CO₂H, S—(C₁₋₆)alkyl, CON((C₁₋₆)alkyl)₂; (C₁₋₄)alkylCO₂H, (C₁₋₆)alkylCO₂(C₁₋₆)alkyl, C(═O)—(C₁₋₆)alkyl, C(═O)-phenyl (wherein said phenyl is optionally substituted with from 1- to 3-groups selected from halogen, (C₁₋₃)alkyl or (C₁₋₃)alkoxy), O—(C₁₋₄)alkyl-N((C₁₋₆)alkyl)₂; O—(C₁₋₄)alkyl-heterocyclyl (wherein said heterocyclyl is optionally substituted with from 1- to 3-groups selected from halogen, (C₁₋₃)alkyl or (C₁₋₃)alkoxy), O—(C₁₋₄)alkyl-heteroaryl (wherein said heteroaryl is optionally substituted with from 1- to 3-groups selected from halogen, (C₁₋₃)alkyl or (C₁₋₃)alkoxy), O—(C₁₋₄)alkyl-phenyl (wherein said phenyl is optionally substituted with from 1- to 3-groups selected from halogen, (C₁₋₃)alkyl or (C₁₋₄)alkoxy) and (C₁₋₆)alkylN((C₁₋₆)alkyl)₂. Some non-limiting examples of “heteroaryl” rings as used herein include furanyl, thiophenyl, pyrrolyl, imadozyl, pyrazolyl, triazolyl, tetrazolyl, thiazolyl, oxazolyl, isoxazolyl, pyridyl, pyridazyl, pyrazinyl, quinolinyl, isoquinolinyl, benzofuranyl, benzothiophenyl, indolyl, indazolyl, phenoxazine, and substituted versions thereof.

As used herein, the term “heterocyclyl” refers to a monocyclic, bicyclic or tricyclic ring system ring system having a total of from five- to seventeen-backbone atoms, wherein none of the rings is aromatic, and each ring contains from five- to seven-backbone atoms. The “heterocyclyl” ring system may contain one or more sites of unsaturation and must contain from one- to four-heteroatoms in the backbone independently selected from N, O and S. When a backbone sulfur or sulfurs are present, each sulfur may be independently present as S, SO or SO₂. When a nitrogen atom or nitrogen atoms are present in the backbone, each nitrogen atom is optionally and independently substituted with (C₁₋₆)alkyl, (C₁₋₃)alkylaryl (wherein said aryl is phenyl and is optionally substituted with from 1- to 3-groups selected from halogen, (C₁₋₃)alkyl and (C₁₋₃)alkoxy), C(═O)—(C₁₋₆)alkyl, CO₂—(C₁₋₆)alkyl, CO₂—(C₁₋₃)alkylaryl (wherein said aryl is phenyl and said phenyl is optionally substituted with 1- to 3-substituents independently selected from halogen, (C₁₋₃)alkyl and (C₁₋₃)alkoxy), (C═O)-phenyl (wherein said phenyl is optionally substituted with 1- to 3-substituents selected from halogen, (C₁₋₃)alkyl and (C₁₋₃)alkoxy). Unless otherwise specified, the “heteroaryl” ring may be further substituted with 1- to 5-groups selected from halogen, (C₁₋₆)alkyl, (C₁₋₆)alkoxy, (C₁₋₆)haloalkyl, oxo, hydroxyl, ((C₁₋₆)alkyl)₂N, CO₂(C₁₋₆)alkyl, CHO, CO₂H, S—(C₁₁)alkyl, CON((C₁₋₆)alkyl)₂; (C₁₋₆)alkylCO₂H, (C₁₋₆)alkylCO₂(C₁₋₁₆)alkyl, C(═O)—(C₁₋₆)alkyl, C(═O)-phenyl (wherein said phenyl is optionally substituted with from 1- to 3-groups selected from halogen, (C₁₋₃)alkyl or (C₁₋₃)alkoxy), O—(C₁₋₄)alkyl-N((C₁)alkyl)₂; O—(C₁₋₄)alkyl-heterocyclyl (wherein said heterocyclyl is optionally substituted with from 1- to 3-groups selected from halogen, (C₁₋₃)alkyl or (C₁₋₃)alkoxy), O—(C₁₋₄)alkyl-heteroaryl (wherein said heteroaryl is optionally substituted with from 1- to 3-groups selected from halogen, (C₁₋₃)alkyl or (C₁₋₃)alkoxy), O—(C₁₋₄)alkyl-phenyl (wherein said phenyl is optionally substituted with from 1- to 3-groups selected from halogen, (C₁₋₃)alkyl or (C₁₋₃)alkoxy) and (C₁₋₆)alkylN((C₁₋₆)alkyl)₂. Some non-limiting examples of “heterocyclyl” rings as used herein include tetrahydrofuran, pyran, 1,4-dioxane, 1,3-dioxane, piperidine, pyrrolidine, piperazine, morpholine, thiomorpholine, tetrahydrothiophene, tetrahydrothiopyran, pyrazolidine, hexahydroazepine, decahydroquinoline, and substituted versions thereof.

This invention also contemplates the use of “physiologically functional derivatives” of compounds of formula (I), (II), or (III), wherein “physiologically functional derivatives” refers to any acceptable derivative of a compound of the present invention, for example, an ester or amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry and Drug Discovery, 5^{th} Edition, Vol. 1: Principles and Practice, which is herein incorporated to the extent that it teaches physiologically functional derivatives.

As used herein, the term “optionally” means that the subsequently described event(s) may or may not occur, and includes both event(s), which occur, and events that do not occur.

As used herein, the term “solvate” refers to a complex of variable stoichiometry formed by a solute and a compound of this invention, or a salt or physiologically functional derivative thereof, and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds of this invention as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of this invention are considered to be within the scope of this invention.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of this invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

For purposes of this invention, it will be understood that phrases such as “a compound of formula (I), (II), or (III), and pharmaceutically acceptable salts, solvates and functional derivatives thereof” are intended to encompass the compound of formula (I), (II), or (III), a functional derivative of a compound of formula (I), (II), or (III), a solvate of formula (I), (II), or (III) or any pharmaceutically acceptable combination of these. Thus, by way of non-limiting example used here for illustrative purposes the phrase “a compound of formula (I), (II), or (III), and pharmaceutically acceptable salts and solvates thereof” may include a pharmaceutically acceptable salt of formula (I), (II), or (III), a pharmaceutically acceptable solvate of a compound of formula (I), (II), or (III), or a pharmaceutically acceptable solvate of a salt of a compound of formula (I), (II), or (III).

While it is possible that, for use in therapy, therapeutically effective amounts of a compound of this invention, as well as salts, solvates and physiological functional derivatives thereof may be administered as the raw chemical, it is possible to present the active ingredient as a pharmaceutical composition. Accordingly, the invention further provides pharmaceutical compositions, which include therapeutically effective amounts of compounds of this invention and salts, solvates and physiological functional derivatives thereof, and one or more pharmaceutically acceptable carriers, diluents, or excipients. The compounds of this invention and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of this invention or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, or 1 mg to 700 mg, or 5 mg to 100 mg of a compound of the invention, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of this invention for the treatment of cardiovascular disease, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of the compound per se. It is envisaged that similar dosages would be appropriate for treatment of the other conditions referred to above.

The compounds of this invention, and salts, solvates and physiological functional derivatives thereof, are believed to have utility in chronic renal disease, congestive heart failure, and cardiovascular remodeling as a result of inhibition of the protein kinase SGK-1.

The present invention thus also provides compounds and pharmaceutically acceptable salts or solvates thereof, or physiologically functional derivatives thereof, for use in medical therapy, and particularly in the treatment of disorders mediated at least in part by SGK-1 activity.

The SGK-1 activity referred to herein is any SGK-1 activity that is deemed to be desirable to modulate in a mammalian subject. SGK-1 activity may take the form of, for instance, an abnormal increase in activity, or an aberration in the timing and or control of SGK-1 activity. Such inappropriate activity may result then, for example, from overexpression or mutation of the protein kinase leading to inappropriate or uncontrolled activation. Alternatively, SGK-1 activity may be deemed within a normal range but still may be a good candidate for regulation, inhibition or modulation where it is determined that such activities would contribute to a desired result.

The present invention is directed to methods of regulating, modulating, or inhibiting SGK-1 for the prevention and/or treatment of disorders related to unregulated SGK-1 activity. In particular, the compounds of the present invention can also be used in the treatment of certain forms of renal and cardiovascular disease as well as congestive heart failure.

A further aspect of the invention provides a method of treatment of a mammal suffering from a disorder mediated by SGK-1 activity, which includes administering to said subject an effective amount of a compound of the invention or a pharmaceutically acceptable salt, solvate, or a physiologically functional derivative thereof.

A further aspect of the present invention provides the use of a compound of this invention, or a pharmaceutically acceptable salt or solvate thereof, or a physiologically functional derivative thereof, in the preparation of a medicament for the treatment of a disorder characterized by SGK-1 activity.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I), (II), or (III) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons), herein incorporated by reference. These groups are removed at a convenient stage of the compound synthesis using methods that are readily apparent to those skilled in the art. The selection of processes as well as the reaction conditions and order of their execution shall be consistent with the preparation of compounds of Formula (I), (II), or (III). Those skilled in the art will recognize if a stereocenter exists in compounds of Formula (I), (II), or (III). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), herein incorporated by reference.

The compounds of the present invention were prepared by the methods illustrated in Schemes I through VI.

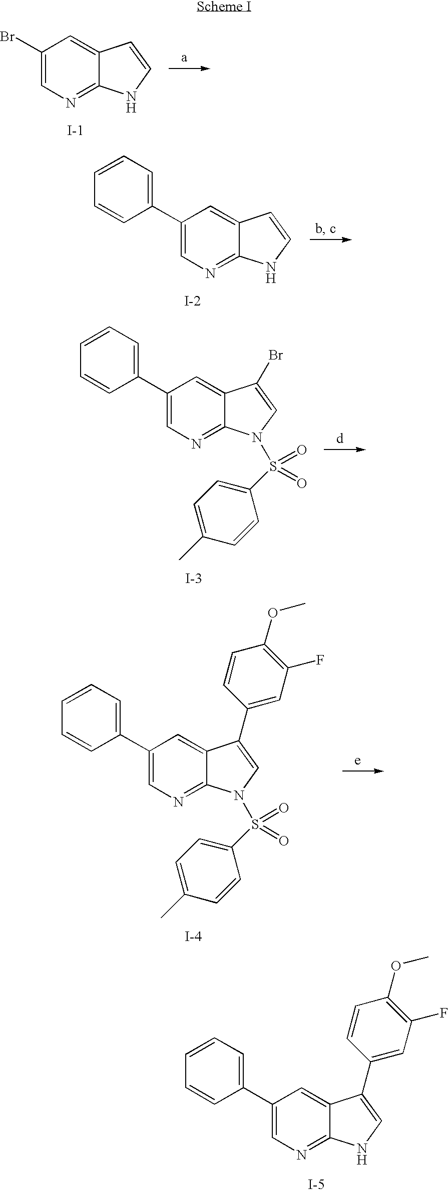

Reagents and Conditions: (a) PhB(OH)₂, PdCl₂(dppf), K₂CO₃, 2.5:1 dioxane/water, 80° C.; (b) Br₂, CHCl₃ or NBS, CHCl₃; (c) TsCl, Bu₄NHSO₄, 6N NaOH/CH₂Cl₂, r.t., 1 h or (i) LDA, THF, −78° C., (ii) TsCl, −78° C.→r.t.; (d) 3-F-4-MeOPhB(OH)₂, PdCl₂(PPh₃)₂, 2M Na₂CO₃, DMF, 90° C. or 3-F-4-MeOPhB(OH)₂, PdCl₂(dppf), K₂CO₃, 2.5:1 dioxane/water, 80° C., 12 h, or 3-F-4-MeOPhB(OH)₂, PdCl₂(dppf), K₂CO₃, 2.5:1 dioxane/water, μν, 150° C., 5 min; (e) 2.5 N NaOH, dioxane, reflux, 1 h or Bu₄NF, THF, 60° C., 30 m.

Azaindole (I-1) (the term azaindole may be used interchangeably in this specification with pyrrolo[2,3-b]pyridine(e)) is reacted with phenylboronic acid or any other suitable boronic acid or boronate under Suzuki coupling conditions to afford 5-aryl or 5-heteroaryl-7-azaindole (I-2). For example, 5-bromo-7-azaindole is coupled with phenylboronic acid using the combination of [1,1′-bis(diphenylphosphino)ferrocene]dichloropalladium(II) and potassium carbonate in a mixture of 2.5:1 dioxane/water at a temperature of about 80° C. to about reflux for about 1 h to 16 h. The Suzuki reaction is well-known to those of skill in the art (for a review, see Suzuki, A. In Modern Arene Chemistry; Astruc, D. Ed.; Wiley-VCH Verlag: Weinheim, Germany, 2002, pp. 53-106, herein incorporated by reference in its entirety). Intermediate (I-2) is brominated in the 3-position with bromine in chloroform or other suitable organic solvent at about 0° C. to about room temperature for about 10 minutes to about 60 minutes, or NBS or a similar commercially available brominating reagent and a base such as triethylamine in an organic solvent such as dichloromethane or THF at about room temperature for about 1 hour to about 16 hours, and the product is treated with p-toluenesulfonyl chloride (tosyl chloride) or a similar commercially available tosyl reagent and a base such as aqueous sodium hydroxide in an organic solvent such as dichloromethane at a temperature of about 0° C. to about room temperature for about 1 hour to about 12 hours to afford (I-3). Alternatively, the tosyl group may be introduced under anhydrous conditions by treatment of the intermediate with a base such as lithium diisopropylamide (LDA) in an organic solvent such as THF at a temperature of about −78° C. to about −40° C. for ten minutes to about 2 hours followed by addition of tosyl chloride at a temperature of about −78° C. to about room temperature for about 30 minutes to about 3 hours. For example, 3-bromo-5-phenyl-7-azaindole hydrobromide was treated with tosyl chloride and tetra-N-butylammonium hydrogen sulfate in a bilayer of dichloromethane and 2N sodium hydroxide to afford 3-bromo-1-[(4-methylphenyl)sulfonyl]-5-phenyl-1H-pyrrolo[2,3-b]pyridine. The use of protecting groups to mask reactive functionality is well-known to those of skill in the art, and other protecting groups are listed in standard reference volumes, such as Greene, “Protective Groups in Organic Synthesis” (published by Wiley-Interscience), herein incorporated by reference in its entirety. Suzuki reaction of I-3 with an appropriate aryl- or heteroarylboronic acid or boronate affords intermediate (I-4). For example, 3-bromo-1-[(4-methylphenyl)sulfonyl]-5-phenyl-1H-pyrrolo[2,3-b]pyridine (I-3) is coupled with 3-fluoro-4-methoxyphenylboronic acid in the presence of bis(triphenylphosphine)dichloropalladium(II) and sodium carbonate in DMF at a temperature of about 70° C. to about 110° C. for about 6 hours to about 24 hours to afford 3-[3-fluoro-4-(methyloxy)phenyl]-1-[(4-methylphenyl)sulfonyl]-5-phenyl-1H-pyrrolo[2,3-b]pyridine (1-4). Removal of the tosyl protecting group is accomplished by treatment of the intermediate (1-4) with either aqueous base and a cosolvent such as dioxane at reflux or tetra-N-butylammonium fluoride in a suitable organic solvent such as THF at 60-90° C. for a time of about 30 minutes to about 12 hours. For example, 3-[3-fluoro-4-(methyloxy)phenyl]-1-[(4-methylphenyl)sulfonyl]-5-phenyl-1H-pyrrolo[2,3-b]pyridine (1-4) is treated with tetra-N-butylammonium fluoride in THF at 60° C. for 30 minutes to afford 3-[3-fluoro-4-(methyloxy)phenyl]-5-phenyl-1H-pyrrolo[2,3-b]pyridine (1-5).

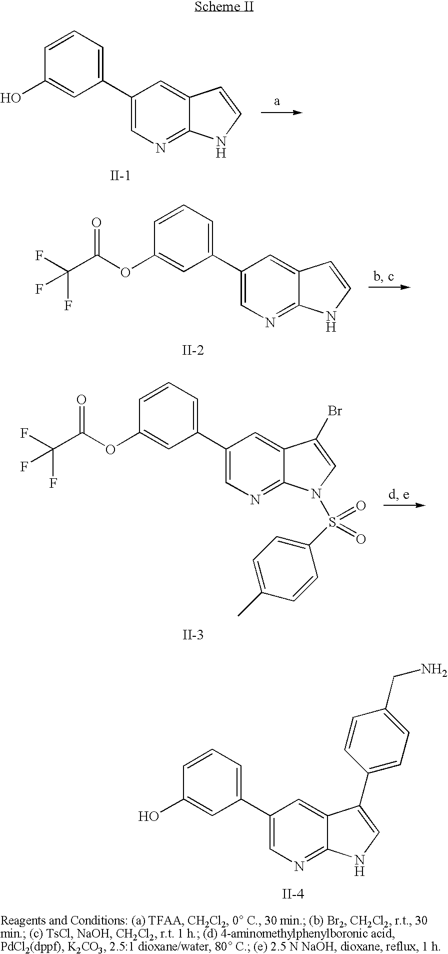

Certain protecting groups beyond the N-1 protecting group of the 7-azaindole core may be required to complete the synthesis of the compounds of interest. Those groups requiring protection would be recognizable to those skilled in the art and the choice of protecting group would be made based on the nature of the functionality to be protected. For example, Scheme II shows that the protection of the intermediate (II-1) can be accomplished using trifluoroacetic anhydride or any other commercially available trifluoroacetylating agent in an organic solvent such as dichloromethane at a temperature of about −10° C. to about 10° C. for about 10 minutes to about 6 h. The remainder of the synthetic scheme then follows the same general route as outlined in Scheme I. It should be noted that there may or may not be additional steps required to remove the protecting group prior to isolation of the compound of interest. Those steps would be recognizable to those skilled in the art.

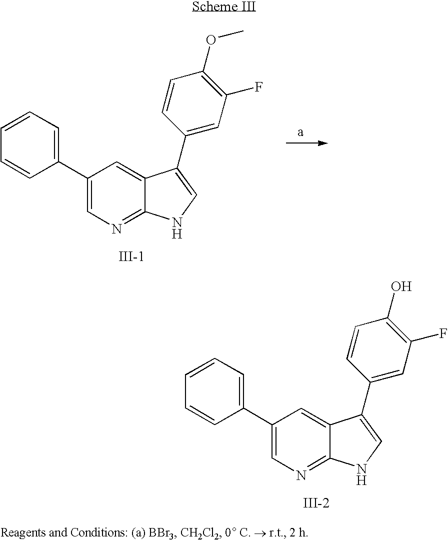

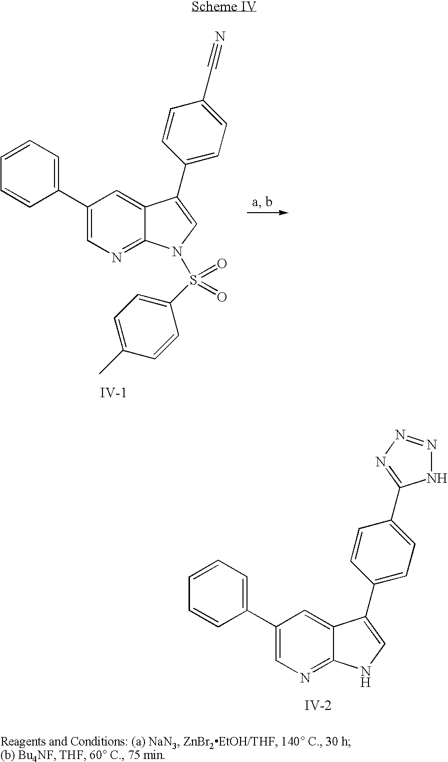

Further manipulations of the functional groups on the aryl rings at either the 3- or the 5-position of the azaindole core may be necessary to complete the synthesis of the compounds of interest. These modifications can take place either before or after the removal of the tosyl protecting group at the 1-position. For example, compound of interest (III-1) could be further manipulated to compound of interest (III-2). In another example, intermediate (IV-1) undergoes functional group manipulation and subsequent protecting group removal to provide compound of interest (IV-2). Such functional group transformations, conditions to effect such functional group transformations, and stages at which the transformations are best carried out are known to those skilled in the art.

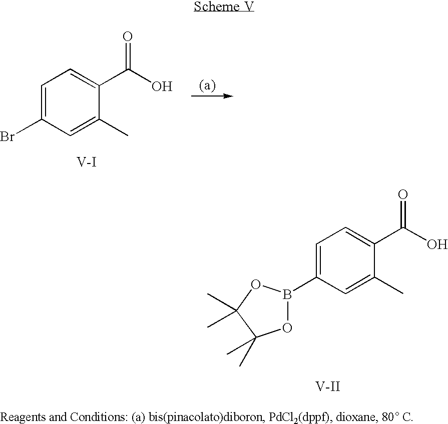

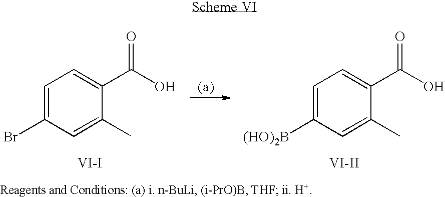

Certain boronate esters or boronic acids may require preparation as intermediates. Conversion to the pinacol boronate ester can be accomplished by palladium-catalyzed coupling of the intermediate aryl bromide or iodide with bis(pinacolato)diboron, as exemplified by the conversion of intermediate (V-I) to (V-II) (see J. Org. Chem. 1995, 60, 7508; J. Org. Chem. 2003, 68, 3729.). Alternatively, direct conversion to the boronic acid can be carried out by trapping an arylmetal intermediate, generated by treating an aryl bromide or iodide with a reagent such as n-butyllithium, with a trialkylborate such as trimethylborate or triisopropylborate followed by acidic workup as exemplified by the conversion of intermediate (VI-I) to boronic acid (VI-II) in Scheme VI (see J. Med. Chem. 2000, 43, 517.). Such functional group transformations, conditions to effect such functional group transformations, and stages at which the transformations are best carried out are known to those skilled in the art.

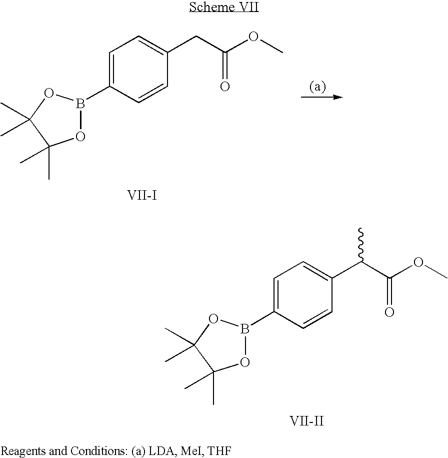

Certain boronate esters or boronic acid intermediates may require modification. For example, treatment of boronate ester (VII-I) with lithium diisopropylamide (LDA) followed by addition of iodomethane provides the intermediate (VII-I). Such functional group transformations, conditions to effect such functional group transformations, and stages at which the transformations are best carried out are known to those skilled in the art.

### EXAMPLES

### General

Proton nuclear magnetic resonance (¹H NMR) spectra were recorded at 400 MHz, and chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform, DMSO-d₆ is hexadeuteriodimethylsulfoxide, and CD₃OD or d₄-CH₃OH is tetradeuteriomethanol. Mass spectra were obtained using electrospray (ES) or atmospheric pressure chemical ionization (APCI) techniques. E. Merck Silica Gel 60 F-254 thin layer plates were used for thin layer chromatography. Flash chromatography was carried out on E. Merck Kieselgel 60 (230-400 mesh) silica gel or on an ISCO Combi-flash purification system using pre-filled silica gel cartridges. Preparative HPLC was performed using Gilson chromatography systems using a 30×100 mm Xterra Prep RP column at a flow rate of 40 mL/min. The solvent system used was a variable gradient of 18% to 90% acetonitrile/water using either 0.1% TFA or ammonium hydroxide to adjust the pH to 10. Celite® is a filter aid composed of acid-washed diatomaceous silica, and is a registered trademark of Manville Corp., Denver, Colo.

### Preparations

### Preparation 1. Methyl 2-methyl-2-[4-(4,4,5,5-tetramethyl-1,3,2-dioxaborolan-2-yl)phenyl]propanoate

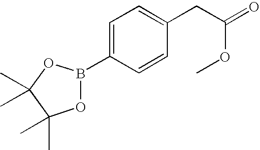

### a) Methyl[4-(4,4,5,5-tetramethyl-1,3,2-dioxaborolan-2-yl)phenyl]acetate

To a solution of [4-(4,4,5,5-tetramethyl-1,3,2-dioxaborolan-2-yl)phenyl]acetic acid (1 g, 3.8 mmol) in MeOH (19 mL) was added 2 drops of conc. HCl and the mixture was stirred at room temperature for 12 h. Solvent was removed and the residue was extracted with CH₂Cl₂. The organic layer was washed with sat. NaHCO₃ and concentrated to give the product as a colorless oil (1 g, 95%). ¹H-NMR (CDCl₃) δ: 1.359 (12H, s), 3.666 (2H, s), 3.703 (3H, s), 7.313 (2H, d, J=7.6 Hz), 7.08 (2H, d, J=7.6 Hz).

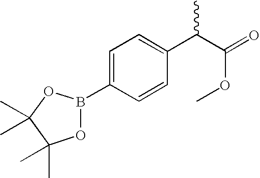

### b) Methyl 2-[4-(4,4,5,5-tetramethyl-1,3,2-dioxaborolan-2-yl)phenyl]propanoate

To a solution of methyl[4-(4,4,5,5-tetramethyl-1,3,2-dioxaborolan-2-yl)phenyl]acetate (1.04 g, 3.78 mmol) in THF (6 mL) was added LDA (3.5 mL, 1.5M in THF) dropwise at −78° C. under argon. After stirring for 20 min, the reaction mixture was warmed up to 0° C., MeI (3.22 g, 22.6 mmol) was added and the mixture was stirred for 1 h at room temperature. The reaction was quenched by adding HCl (2M solution) until pH˜2. The mixture was extracted with EtOAc, concentrated and purified via combiflash to give the product as a white solid (0.77 g, 70%). ¹H-NMR (CDCl₃) δ: 1.354 (12H, s), 1.517 (3H, d, J=7.2 Hz), 3.667 (3H, s), 3.750 (1H, m), 7.323 (2H, d, J=7.2 Hz), 7.796 (2H, d, J=7.2 Hz).

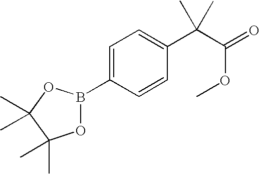

### a) Methyl 2-methyl-2-[4-(4,4,5,5-tetramethyl-1,3,2-dioxaborolan-2-yl)phenyl]propanoate

To a solution of methyl 2-(4-bromophenyl)-2-methylpropanoate (3 g, 11.67 mmol) and bis(pinacolato)diboron (4.45 g, 17.5 mmol) in dioxane (42 mL) was added Pd(dppf)Cl₂ (0.29 g, 0.36 mmol) and KOAc (3.44 g, 35 mmol). The reaction mixture was stirred at 95° C. for 8 h. After cooling to room temperature, EtOAc and brine were added and the mixture was filtered through Celite. The layers were separated and the organic layer was concentrated and purified by flash column chromatography. The crude product was then recrystallized with hot hexane to give the pure product (2.08 g, 59%) as colorless crystals. MS (ES) m/e 305.4 [M+H]⁺

### Preparation 2,4-(dihydroxyboranyl)-2-ethylbenzoic acid

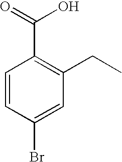

### a) 4-bromo-2-ethylbenzoic acid

To a solution of 4-bromo-2-fluorobenzoic acid (2 g, 9.13 mmol) in THF (15 mL) at 0° C. was added ethylmagnesium bromide (1M in THF, 32.0 mL, 32.0 mmol) slowly using an addition funnel. The reaction was stirred four hours, and then 25 mL of 2N HCl was added slowly to the reaction at 0° C. Ethyl acetate (30 mL) was added, and the layers were separated. NaOH (30 mL, 2.5 N) was added to the organic layer with stirring for 30 minutes. The layers were separated, and the aqueous layer was washed with ethyl acetate (10 mL). The aqueous layer was acidified to pH 2-3 with 6 N HCl. The aqueous layer was extracted three times with ethyl acetate (30 mL). The combined organic extracts were dried over Na₂SO₄, filtered, and concentrated to give the title compound as a white solid (0.575 g, 28%). ¹H NMR (400 MHz, CDCl₃) δ 7.93 (d, J=8.4 Hz, 1H), 7.50 (d, J=2.0 Hz, 1H), 7.45 (dd, J=2.0, 8.8 Hz, 1H), 3.07 (q, J=7.6 Hz, 2H), 1.29 (t, J=7.6 Hz, 3H). LCMS m/e 229.2 [M]⁺.

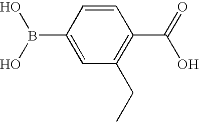

### b) 4-(dihydroxyboranyl)-2-ethylbenzoic acid

To a solution of 4-bromo-2-ethylbenzoic acid (0.250 g, 1.09 mmol) in THF (18 mL) was added triisopropyl borate (3.52 mL, 15.3 mmol). The mixture was cooled to −78° C. and n-butyllithium (2.5 M in hexanes, 6.1 mL, 15.3 mmol) was added slowly over ten minutes. The reaction temperature was maintained at −78° C. for three hours. The reaction was warmed to 0° C. and quenched with 10 mL of 2N HCl. The aqueous layer was extracted twice with ethyl acetate (15 mL). The organic layer was stirred with 2.5 N NaOH (10 mL) for ten minutes. The layers were separated, and the aqueous layer was acidified to pH 3 with 6N HCl. The aqueous layer was extracted twice with ethyl acetate (15 mL). The combined organic extracts were dried over Na₂SO₄, filtered, and concentrated. The crude material was stirred in CH₂Cl₂. This suspension was filtered to obtain the title compound as a white solid (0.120 g, 57%). ¹H NMR (400 MHz, MeOD) δ 7.82 (m, 1H), 7.50 (m, 2H), 3.00 (q, J=7.2 Hz, 2H), 1.24 (t, J=7.6 Hz, 3H). LCMS m/e 194.2 [M+H]⁺.

### Preparation 3: 2-(2-methylpropyl)-4-(4,4,5,5-tetramethyl-1,3,2-dioxaborolan-2-yl)benzoic acid

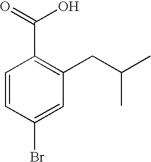

### a) 4-bromo-2-(2-methylpropyl)benzoic acid

This compound was prepared according to the procedure for 4-bromo-2-ethylbenzoic acid in Preparation 1(a).

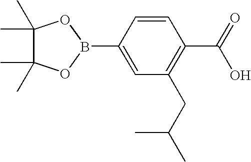

### b) 2-(2-methylpropyl)-4-(4,4,5,5-tetramethyl-1,3,2-dioxaborolan-2-yl)benzoic acid

To a solution of 4-bromo-2-(2-methylpropyl)benzoic acid (2.32 g, 9.02 mmol) in dioxane (120 mL) was added bis(pinacolato)diboron (2.75 g, 10.8 mmol), potassium acetate (2.66 g, 27.1 mmol), and [1,1′-bis(diphenylphosphino)ferrocene]dichloropalladium(II) complex with dichloromethane (0.22 g, 0.27 mmol). The mixture was heated at 80° C. for 18 hours. The reaction was cooled to room temperature and partitioned between ethyl acetate and water (50 mL each). Then 30 mL of 1 N HCl was added and the mixture was filtered through celite. The filter cake was washed with ethyl acetaete:water and the filtrate layers were separated. The aqueous layer was extracted twice with ethyl acetate (50 mL). The combined organic extracts were dried over Na₂SO₄, filtered, and concentrated. The crude mixture was stirred in 10 mL of hexanes for thirty minutes. Immediately, a solid precipitated, and the suspension was filtered. The tan solid (0.614 g) was product. The filtrate was stirred overnight and a solid slowly precipitated. The suspension was filtered to give 0.568 g product for a total of 1.18 g product (43%). ¹H NMR (400 MHz, CDCl₃) δ 8.00 (d, J=7.6 Hz, 1H), 7.72 (d, J=7.6 Hz, 1H), 7.68 (s, 1H), 2.95 (d, J=6.8 Hz, 2H), 1.95 (m, 1H), 1.39 (s, 12H), 0.94 (d, J=6.4 Hz, 6H). LCMS m/e 305.0 [M+H]⁺.

### Preparation 4: 2-(methylamino)-4-(4,4,5,5-tetramethyl-1,3,2-dioxaborolan-2-yl)benzoic acid

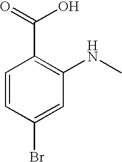

### a) 4-bromo-2-(methylamino)benzoic acid

To a solution of methylamine (2M in THF, 12.8 mL, 25.5 mmol) in THF (23 mL) at 0° C. was added n-butyllithium (2.5M in hexanes, 8.03 mL, 20.1 mmol) slowly. The mixture was stirred for one hour at 0° C. and then it was transferred via cannula to a solution of 4-bromo-2-fluorobenzoic acid (0.5 g, 2.28 mmol) in THF (5 mL) at −78° C. The reaction was stirred for thirty minutes before it was quenched at −78° C. with 27 mL of 1 N HCl. The aqueous layer was extracted four times with ethyl acetate (20 mL). The combined organic extracts were dried over Na₂SO₄, filtered, and concentrated. The crude product was purified using flash silica chromatography (0-7% MeOH/CH₂Cl₂) to give the title compound as a light orange solid (0.183 g, 35%). ¹H NMR (400 MHz, CDCl₃) δ 7.82 (d, J=8.8 Hz, 1H), 6.86 (d, J=1.2 Hz, 1H), 6.76 (dd, J=1.6, 8.4 Hz, 1H), 2.94 (s, 3H). LCMS m/e 229.8 [M]⁺.

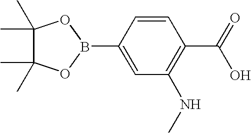

### b) 2-(methylamino)-4-(4,4,5,5-tetramethyl-1,3,2-dioxaborolan-2-yl)benzoic acid

This compound was prepared according to the procedure for 2-(2-methylpropyl)-4-(4,4,5,5-tetramethyl-1,3,2-dioxaborolan-2-yl)benzoic acid in Preparation 2(b). LCMS m/e 277.2 [M]⁺.

### Preparation 5: 4-(dihydroxyboranyl)-2-(dimethylamino)benzoic acid

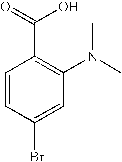

### a) 4-bromo-2-(dimethylamino)benzoic acid

This compound was prepared according to the procedure for 4-bromo-2-(methylamino)benzoic acid in Preparation 3(a). LCMS m/e 244.2 [M]⁺.

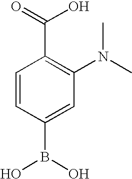

### b) 4-(dihydroxyboranyl)-2-(dimethylamino)benzoic acid

This compound was prepared according to the procedure for 2-(methylamino)-4-(4,4,5,5-tetramethyl-1,3,2-dioxaborolan-2-yl)benzoic acid in Preparation 3(b) with the following modification. The product, 4-(dihydroxyboranyl)-2-(dimethylamino)benzoic acid, precipitated from the aqueous layer during the extraction. The aqueous layer was filtered to give the title compound as a grey solid (68%). ¹H NMR (400 MHz, DMSO-d₆) δ 8.38 (s, 2H), 8.06 (s, 1H), 7.95 (d, J=7.6 Hz, 1H), 7.77 (d, J=7.6 Hz, 1H), 2.83 (s, 6H). LCMS m/e 210.0 [M+H]⁺.

### Preparation 6. 2-methyl-4-(4,4,5,5,-tetramethyl-1,3,2-dioxaborolan-2-yl)benzoic acid

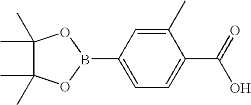

Prepared according to the procedure given in Preparation 2(b) starting with 4-bromo-2-methylbenzoic acid. LCMS m/e 263.2 [M+H]⁺.

### Preparation 7. 2,6-dimethyl-4-(4,4,5,5.-tetramethyl-1,3,2-dioxaborolan-2-yl)benzoic acid

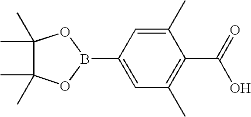

Prepared according to the procedure given in Preparation 2(b) starting with 4-bromo-2,6-dimethylbenzoic acid (J. Am. Chem. Soc. 1941, 63,1679). LCMS m/e 276.2 [M+H]⁺.

### Preparation 8. 2-(1,1-dimethylethyl)-4-(4,4,5,5-tetramethyl-1,3,2-dioxaborolan-2-yl)benzoic acid

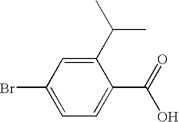

### a) 4-bromo-2-(2-propyl)benzoic acid

Prepared according to the procedure given in Preparation 1(a) starting with 4-bromo-2-fluorobenzoic acid. LCMS m/e 244.2 [M+H]⁺.

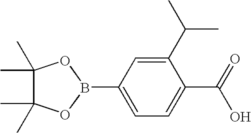

### b) 2-(1,1-dimethylethyl)-4-(4,4,5,5-tetramethyl-1,3,2-dioxaborolan-2-yl)benzoic acid

Prepared according to the procedure given in Preparation 2(b) starting with 4-bromo-2-(2-propyl)benzoic acid. LCMS m/e 291.0 [M+H]⁺.

### Preparation 9. 1-(1,1-dimethylethyloxycarbonyl)-6-(4,4,5,5-tetramethyl-1,3,2-dioxaborolan-2-yl)indole

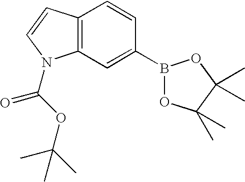

tert-Butyl pyrocarbonate (988 mg, 4.52 mmol) was added in one portion to a solution of 6-(4,4,5,5-tetramethyl-1,3,2-dioxaborolan-2-yl)indane (1.0 g, 4.11 mmol), DMAP (5 mg), and N,N-diisopropylethylamine (1.4 mL, 1.1 g, 8.23 mmol) in dichloromethane (15 mL). The reaction mixture was stirred at room temperature for 17 h and then diluted with 1:1 ethyl acetate/hexanes (50 mL). The resultant mixture was washed with 1 N HCl (20 mL) and saturated aqueous sodium chloride (25 mL). The organics were dried over anhydrous sodium sulfate and were concentrated. The residue was purified by flash column chromatography on silica gel (dichloromethane grading to 10% ethyl acetate in dichloromethane) to afford 1-(1,1-dimethylethyloxycarbonyl)-6-(4,4,5,5-tetramethyl-1,3,2-dioxaborolan-2-yl)indole (1.22 g, 87%) as a yellow oil. MS m/e 344 [M+H]⁺.

### Preparation 10. 1-(1,1-dimethylethyloxycarbonyl)-6-(4,4,5,5-tetramethyl-1,3,2-dioxaborolan-2-yl)indazole and 2-(1,1-dimethylethyloxycarbonyl)-6-(4,4,5,5-tetramethyl-1,3,2-dioxaborolan-2-yl)indazole

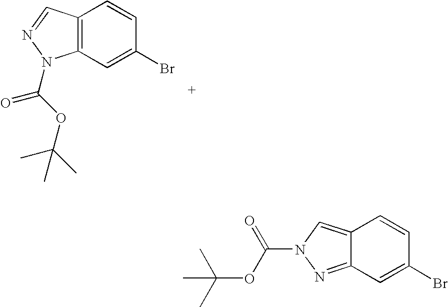

### a) 1-([1,1-dimethylethyl]oxycarbonyl)-6-bromoindazole and 2-([1,1-dimethylethyl]oxycarbonyl)-6-bromoindazole

Prepared as described in Preparation 8 starting with 6-bromoindazole.

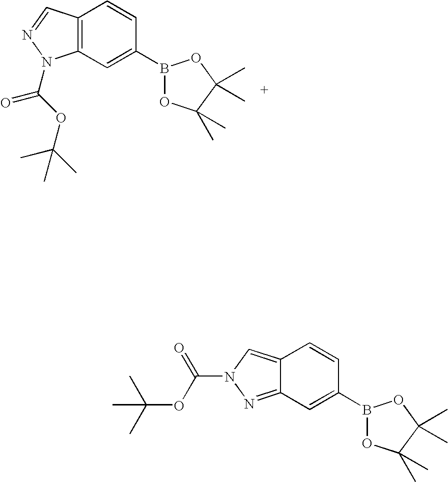

### b) 1-(1,1-dimethylethyloxycarbonyl)-6-(4,4,5,5-tetramethyl-1,3,2-dioxaborolan-2-yl)indazole and 2-(1,1-dimethylethyloxycarbonyl)-6-(4,4,5,5-tetramethyl-1,3,2-dioxaborolan-2-yl)indazole

Prepared as described in Preparation 2(b) starting with 1-(1,1-dimethylethyloxycarbonyl)-6-bromoindazole and 2-(1,1-dimethylethyloxycarbonyl)-6-bromoindazole to afford 1-(1,1-dimethylethyloxycarbonyl)-6-(4,4,5,5-tetramethyl-1,3,2-dioxaborolan-2-yl)indazole and 2-(1,1-dimethylethyloxycarbonyl)-6-(4,4,5,5-tetramethyl-1,3,2-dioxaborolan-2-yl)indazole as a mixture. MS m/e 289 [M-t-Bu]⁺.

### Preparation 11. 2-chloro-4-(4,4,5,5-tetramethyl-1,3,2-dioxaborolan-2-yl)benzoic acid

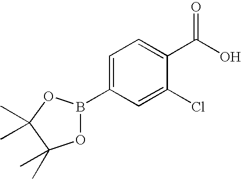

To a solution of 4-bromo-2-chlorobenzoic acid (0.15 g, 0.64 mmol) and bis(pinacolato)diboron (0.19 g, 0.76 mmol) in dioxane (30 mL) was added [1,1′-Bis(diphenylphosphino)ferrocene]dichloropalladium(II) complex with dichloromethane (0.015 g, 0.019 mmol) and potassium acetate (0.18 g, 1.92 mmol). The reaction mixture was stirred at 80° C. for 5 h. After cooling to room temperature, ethyl acetate (10 mL) and water (10 mL) were added. The aqueous layer was extracted with ethyl acetate (2×20 mL). The organic portions were concentrated and recrystallized with hot hexanes to give the pure product (0.13 g, 69%). MS (ES) m/e 283 [M+H]⁺

### Preparation 12. 4-(4,4,5,5-tetramethyl-1,3,2-dioxaborolan-2-yl)2,6-bis(trifluoromethyl)benzoic acid

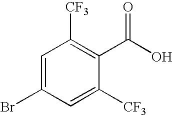

### a) 4-bromo-2,6-bis(trifluoromethyl)benzoic acid

A solution of 1,3-dibromo-5,5-dimethylhydantoin (1.1 g, 3.85 mmol) in concentrated sulfuric acid (4 mL) was cooled to 0° C. This was followed by the addition of 2,6-bis(trifluoromethyl)benzoic acid (1.0 g, 3.87 mmol). The resultant slurry was stirred at 0° C. for 2 h, followed by stirring overnight at room temperature. The reaction mixture was poured into ice water and stored at −5° C. for 2 h. A white precipitate formed which was isolated and dried to yield 4-bromo-2,6-bis(trifluoromethyl)benzoic acid (1.02 g, 78%).

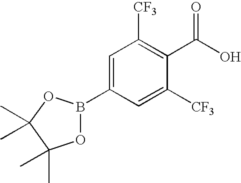

### b) 4-(4,4,5,5-tetramethyl-1,3,2-dioxaborolan-2-yl)2,6-bis(trifluoromethyl)benzoic acid

To a solution of 4-bromo-2,6-bis(trifluoromethyl)benzoic acid (0.5 g, 1.48 mmol) and bis(pinacolato)diboron (0.45 g, 1.78 mmol) in dioxane (20 mL) was added Pd(dppf)Cl₂ (0.036 g, 0.044 mmol) and KOAc (0.43 g, 4.44 mmol). The reaction mixture was heated in the microwave at 150° C. for 20 min. After cooling, EtOAc (10 mL) and brine (5 mL) were added and the mixture was filtered through celite. The filtrate was acidified and the product was isolated by filtration. Recrystallization from hexanes provided 4-(4,4,5,5-tetramethyl-1,3,2-dioxaborolan-2-yl)2,6-bis(trifluoromethyl)benzoic acid (0.2 g, 32%). LC-MS m/e 385 (M+H)⁺.

### Preparation 13. 2-(azidomethyl)-4-(4,4,5,5-tetramethyl-1,3,2-dioxaborolan-2-yl)methyl benzoate

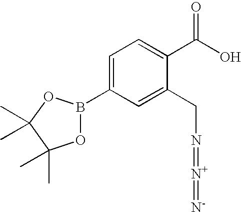

Methyl 4-bromo-2-methylbenzoate (1.4 g, 6.1 mmol) was dissolved in carbon tetrachloride (10 mL). N-Bromosuccinimide (1.19 g, 6.7 mmol) and AlBN (0.02 g, 0.12 mmol) were added and the resultant reaction mixture was refluxed for 5 h. The mixture was cooled to room temperature, washed with water (2×50 mL) and the organics were dried and concentrated. The residue was dissolved in DMF (3 mL), sodium azide (0.15 g, 2.33 mmol) was added and the mixture was heated at 100° C. for 2 h. The reaction mixture was cooled to room temperature and washed with water (2×10 mL). The organics were dried and concentrated. The residue was dissolved in dioxane (10 mL), and bis(pinacolato)diboron (0.51 g, 2.03 mmol) was added followed by Pd(dppf)Cl₂ (0.041 g, 0.05 mmol) and KOAc (0.49 g, 5.07 mmol). The reaction mixture was stirred at 80° C. for 8 h. After cooling down to room temperature, the reaction mixture was washed with water (20 mL) and saturated aqueous sodium chloride (10 mL). The organics were dried and purified by silica gel chromatography (100% hexanes grading to 100% ethyl acetate over 30 minutes) to give 2-(azidomethyl)-4-(4,4,5,5-tetramethyl-1,3,2-dioxaborolan-2-yl)methyl benzoate (0.42 g, 78%).

### Preparation 14. 2-[3,5-difluoro-4-(methyloxy)phenyl]-4,4,5,5-tetramethyl-1,3,2-dioxaborolane

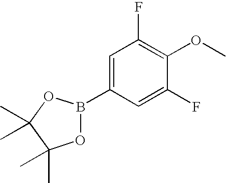

A solution of 4-bromo-2,6-difluoroanisole (223 mg, 1 mmole), bis(pinacaolato)diboron (275 mg, 1.09 mmole), [1,1′-bis(diphenylphosphino)ferrocene]dichloropalladium(II) (40 mg) and KOAc (300 mg) in 5 ml of dioxane was heated in the microwave at 1500 for 20 minutes. The reaction was diluted with H₂O and extracted with Et₂O. The extracts were washed with H₂O, dried and evaporated. The residue was chromatographed on a Florisil column, and the titled compound was eluted with Et₂O, 158 mg (58%). ¹H NMR (400 MHz, CDCl₃) δ 7.32 (d, J=8 Hz, 2H), 4.05 (s, 3H), 1.35 (s, 12H).

### Example 1

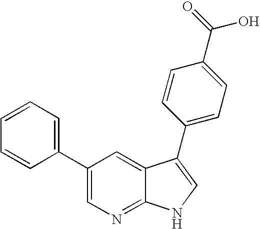

### Preparation of 4-(5-phenyl-1H-pyrrolo[2,3-b]pyridin-3-yl)-benzoic acid

### b) 5-Phenyl-1H-pyrrolo[2,3-b]pyridine

[1,1′-Bis(diphenylphosphino)ferrocene]dichloropalladium(II) (1.04 g, 1.27 mmol, 0.05 equiv) was added in one portion to a suspension of 5-bromo-1H-pyrrolo[2,3-b]pyridine ((5.00 g, 25.4 mmol, 1 equiv), phenylboronic acid (3.70 g, 30.5 mmol, 1.2 equiv), and potassium carbonate (10.5 g, 76.1 mmol, 3 equiv) in 2.5:1 dioxane/water (253 mL). The reaction mixture was placed under N₂ atmosphere and heated in an oil bath set to 80° C. After heating for 22.5 hours, the reaction mixture was cooled to room temperature, acidified with 6N HCl, and partitioned between ethyl acetate (100 mL) and water (100 mL). The mixture was filtered through a pad of pressed Celite, and the layers of the filtrate were separated. The aqueous layer was extracted with ethyl acetate (2 50-mL portions). The combined organics were washed with saturated aqueous sodium chloride (100 mL), dried over sodium sulfate and concentrated. The residue was dissolved in methanol (200 mL), 15 g DOWEX 50WX2-400 ion exchange resin were added, and the mixture was stirred gently for 3 hours. The resin was collected by filtration and washed with methanol (2 100-mL portions), dichloromethane (100 mL), and methanol (100 mL). The product was released from the resin by washing with 4N ammonia in methanol (3 100-mL portions). The 4N ammonia/methanol washings were concentrated in vacuo to provide 5-phenyl-1H-pyrrolo[2,3-b]pyridine as a light brown solid (4.86 g, 99%). ¹H NMR (400 MHz, CDCl₃) δ 10.33 (br s, 1H), 8.62 (s, 1H), 8.22 (s, 1H), 7.66 (d, J=8 Hz, 2H), 7.52 (t, J=7.6 Hz, 2H), 7.43 (m, 2H), 6.62 (s, 1H). LC-MS (ES) m/e 195 (M+H)⁺.

### b) 3-Bromo-5-phenyl-1H-pyrrolo[2,3-b]pyridine

Bromine (1.27 mL, 3.95 g, 24.7 mmol, 1 equiv) was added over a period of 35 minutes to a solution of 5-phenyl-1H-pyrrolo[2,3-b]pyridine (4.8 g, 24.7 mmol, 1 equiv) in chloroform (247 mL). The reaction mixture was stirred at room temperature for 15 min and then concentrated in vacuo. The pale orange foam containing 3-bromo-5-phenyl-1H-pyrrolo[2,3-b]pyridine was carried directly to the next reaction without further purification.

### c) 3-Bromo-1-[(4-methylphenyl)sulfonyl]-5-phenyl-1H-pyrrolo[2,3-b]pyridine

Tetrabutylammonium hydrogen sulfate (100 mg, catalytic) was added to a mixture of 3-bromo-5-phenyl-1H-pyrrolo[2,3-b]pyridine (24.7 mmol, 1 equiv) and p-toluenesulfonyl chloride (5.65 g, 29.6 mmol, 1.2 equiv) in a bilayer of dichloromethane (308 mL) and 6N NaOH (50 mL). The reaction mixture was stirred for 1 hour and then diluted with water (100 mL). The reaction mixture was filtered through a plug of Celite, and the filtrate layers were separated. The aqueous layer was extracted with dichloromethane (100 mL). The combined organics were dried over sodium sulfate and were concentrated. Purification of the residue by ISCO chromatography (120 g silica column, dichloromethane for 10 min, dichloromethane grading to 2% ethyl acetate/dichloromethane over 10 minutes, 2% ethyl acetate/dichloromethane grading to 10% ethyl acetate/dichloromethane over 1 minute, and 10% ethyl acetate/dichloromethane for 10 minutes) afforded 3-bromo-1-[(4-methylphenyl)sulfonyl]-5-phenyl-1H-pyrrolo[2,3-b]pyridine (7.19 g, 68% over two steps) as a tan solid. ¹H NMR (400 MHz, CDCl₃) δ 8.71 (s, 1H), 8.12 (d, J=8, 2H), 7.97 (s, 1H), 7.84 (s, 1H), 7.60 (d, J=7.2 Hz, 2H), 7.51 (t, J=7.6 Hz, 2H), 7.42 (t, J=6.8 Hz, 1H), 7.32 (d, J=8.4 Hz, 2H), 2.41 (s, 3H). LC-MS (ES) m/e 427 (M+H)⁺.

### (d) 4-[5-phenyl-1-(toluene-4-sulfonyl)-1H-pyrrolo[2,3-b]pyridin-3-yl)-benzoic acid

[1,1′-bis(diphenylphosphino)ferrocene]dichloropalladium(II) complex with dichloromethane (246 mg, 0.30 mmol, 0.05 equiv) was added to a suspension of 3-bromo-1-[(4-methylphenyl)sulfonyl]-5-phenyl-1H-pyrrolo[2,3-b]pyridine (2.57 g, 6.01 mmol, 1 equiv), 4-carboxyphenylboronic acid (1.2 g, 7.21 mmol, 1.2 equiv) and potassium carbonate (2.49 g, 18.0 mmol, 3 equiv) in 2.5:1 dioxane/water (60 mL). The reaction flask was equipped with a water-cooled condenser, and the reaction mixture was heated to reflux under a nitrogen atmosphere. After three hours, the reaction mixture was cooled to room temperature and acidified with concentrated HCl (ca. 8 mL). The mixture was then filtered through a pad of Celite, and the filtrate was partitioned between ethyl acetate (100 mL) and water (100 mL). The layers were separated, and the aqueous layer was further extracted with ethyl acetate (2 50-mL portions). The combined organics were dried over sodium sulfate and were concentrated. The 4-[5-phenyl-1-(toluene-4-sulfonyl)-1H-pyrrolo[2,3-b]pyridin-3-yl)-benzoic acid was used directly in the next step without further purification.

### e) 4-(5-phenyl-1H-pyrrolo[2,3-b]pyridin-3-yl)-benzoic acid

The crude 4-[5-phenyl-1-(toluene-4-sulfonyl)-1H-pyrrolo[2,3-b]pyridin-3-yl)-benzoic acid was taken up in a mixture of methanol (50 mL) and 2.5 N NaOH (20 mL). The reaction mixture was heated at 50 C for 30 minutes and cooled to room temperature. The reaction mixture was acidified with concentrated HCl and then partitioned between ethyl acetate (100 mL) and water (100 mL). The precipitate that formed was collected by filtration and set aside. The aqueous layer was further extracted with ethyl acetate (2 50-mL portions), and the combined organics were dried over anhydrous sodium sulfate and concentrated. The original precipitate was combined with the residue from the organics, and the combination was stirred with 10% methanol in dichloromethane (60 mL) for 30 min. The insolubles were collected by filtration, and the filtrate was concentrated in vacuo and stirred again with 10% methanol in dichloromethane (30 mL). The insolubles were again collected by filtration and combined with the first crop. This process was repeated once more to provide 4-(5-phenyl-1H-pyrrolo[2,3-b]pyridin-3-yl)-benzoic acid (1.13 g, 60%). ¹H NMR (400 MHz, CD₃OD) δ 8.54 (s, 2H), 8.14 (d, J=8 Hz, 2H), 7.87 (m, 3H), 7.73 (d, J=8.8 Hz, 2H), 7.51 (t, J=7.6 Hz, 2H), 7.40 (t, J=7.2 Hz. 1H). LC-MS (ES) m/e 315 (M+H)⁺.

### Examples 2-170

Compounds of Examples 2-170 were prepared following the same general procedure described above for Example 1.

### Example 2

({3-[5-(2-naphthalenyl)-1H-pyrrolo[2,3-b]pyridin-3-yl]phenyl}methyl). LC-MS (ES) m/e 350.2 [M+H]⁺.

### Example 3

4-[5-(2-naphthalenyl)-1H-pyrrolo[2,3-b]pyridin-3-yl]benzoic acid. LC-MS (ES) m/e 365.1 [M+H]⁺

### Example 4

{4-[5-(2-naphthalenyl)-1H-pyrrolo[2,3-b]pyridin-3-yl]phenyl}acetic acid. LC-MS (ES) m/e 379.2 [M+H]⁺.

### Example 5

3-{4-[5-(2-naphthalenyl)-1H-pyrrolo[2,3-b]pyridin-3-yl]phenyl}propanoic acid. LC-MS (ES) m/e 393.2 [M+H]⁺.

### Example 6

{3-[5-(2-naphthalenyl)-1H-pyrrolo[2,3-b]pyridin-3-yl]phenyl}methanol. LC-MS (ES) m/e 351.2 [M+H]⁺.

### Example 7

4-{5-[3-(methyloxy)phenyl]-1H-pyrrolo[2,3-b]pyridin-3-yl}benzoic acid. LC-MS (ES) m/e 345.2 [M+H]⁺.

### Example 8

3-(4-{5-[3-(methyloxy)phenyl]-1H-pyrrolo[2,3-b]pyridin-3-yl}phenyl)propanoic acid. LC-MS (ES) m/e 373.2 [M+H]⁺.

### Example 9

3-{3-[4-(aminomethyl)phenyl]-1H-pyrrolo[2,3-b]pyridin-5-yl}benzonitrile. LC-MS (ES) m/e 325.4 [M+H]⁺.

### Example 10

4-{5-[3-(aminocarbonyl)phenyl]-1H-pyrrolo[2,3-b]pyridin-3-yl}benzoic acid. LC-MS (ES) m/e 358.4 [M+H]⁺.

### Example 11

4-[5-(3-cyanophenyl)-1H-pyrrolo[2,3-b]pyridin-3-yl]benzoic acid. LC-MS (ES) m/e 340.2 [M+H]⁺.

### Example 12

4-{5-[6-(methyloxy)-3-pyridinyl]-1H-pyrrolo[2,3-b]pyridin-3-yl}benzoic acid. LC-MS (ES) m/e 346.2 [M+H]⁺.

### Example 13

3-{3-[3-(aminomethyl)phenyl]-1H-pyrrolo[2,3-b]pyridin-5-yl}benzonitrile. LC-MS (ES) m/e 325.4 [M+H]⁺.

### Example 14

4-[5-(1-naphthalenyl)-1H-pyrrolo[2,3-b]pyridin-3-yl]benzoic acid. LC-MS (ES) m/e 365.2 [M+H]⁺.

### Example 15

2-fluoro-4-[5-(1-naphthalenyl)-1H-pyrrolo[2,3-b]pyridin-3-yl]benzoic acid. LC-MS (ES) m/e 383.2 [M+H]⁺.

### Example 16

3-amino-5-[5-(1-naphthalenyl)-1H-pyrrolo[2,3-b]pyridin-3-yl]benzoic acid. LC-MS (ES) m/e 380.2 [M+H]⁺.

### Example 17

3-{4-[5-(1-naphthalenyl)-1H-pyrrolo[2,3-b]pyridin-3-yl]phenyl}propanoic acid. LC-MS (ES) m/e 393.2 [M+H]⁺.

### Example 18

3-(4-{5-[3-(aminocarbonyl)phenyl]-1H-pyrrolo[2,3-b]pyridin-3-yl}phenyl)propanoic acid. LC-MS (ES) m/e 386.2 [M+H]⁺.

### Example 19

3-{4-[5-(3-cyanophenyl)-1H-pyrrolo[2,3-b]pyridin-3-yl]phenyl}propanoic acid. LC-MS (ES) m/e 368.2 [M+H]⁺.

### Example 20

3-(4-{5-[6-(methyloxy)-3-pyridinyl]-1H-pyrrolo[2,3-b]pyridin-3-yl}phenyl)propanoic acid. LC-MS (ES) m/e 373.6 [M+H]⁺.

### Example 21

{4-[5-(1-naphthalenyl)-1H-pyrrolo[2,3-b]pyridin-3-yl]phenyl}acetic acid. LC-MS (ES) m/e 379.2 [M+H]⁺.

### Example 22

(4-{5-[3-(aminocarbonyl)phenyl]-1H-pyrrolo[2,3-b]pyridin-3-yl}phenyl)acetic acid. LC-MS (ES) m/e 372.2 [M+H]⁺.

### Example 23

{4-[5-(3-cyanophenyl)-1H-pyrrolo[2,3-b]pyridin-3-yl]phenyl}acetic acid. LC-MS (ES) m/e 354.2 [M+H]⁺.

### Example 24

(4-{5-[6-(methyloxy)-3-pyridinyl]-1H-pyrrolo[2,3-b]pyridin-3-yl}phenyl)acetic acid. LC-MS (ES) m/e 360.0 [M+H]⁺.

### Example 25

3-{4-[5-(3-methanesulfonylamino-phenyl)-1H-pyrrolo[2,3-b]pyridin-3-yl]-phenyl}-propionic acid. LC-MS (APCI) m/e 436.4 [M+H]⁺.

### Example 26

(4-[5-(3-methanesulfonylamino-phenyl)-1H-pyrrolo[2,3-b]pyridin-3-yl]-phenyl)-acetic acid. LC-MS (APCI) m/e 422.4 [M+H]⁺.

### Example 27

3-{4-[5-(3-methanesulfonyl-phenyl)-1H-pyrrolo[2,3-b]pyridin-3-yl]-phenyl}-propionic acid. LC-MS (APCI) m/e 421.2 [M+H]⁺.

### Example 28

{4-[5-(3-methanesulfonyl-phenyl)-1H-pyrrolo[2,3-b]pyridin-3-yl]-phenyl}-acetic acid. LC-MS (APCI) m/e 407.6 [M+H]⁺.

### Example 29

3-[3-fluoro-4-(methyloxy)phenyl]-5-phenyl-1H-pyrrolo[2,3-b]pyridine. LC-MS (ES) 473 [M+H]⁺.

### Example 30

5-phenyl-3-pyridin-4-yl-1H-pyrrolo[2,3-b]pyridine. LC-MS e/s 272 [M+H]⁺.

### Example 31

3-(5-phenyl-1H-pyrrolo[2,3-b]pyridin-3-yl)-benzoic acid LC-MS (APCI) m/e 315 [M+H]⁺.

### Example 32

N-[3-(5-phenyl-1H-pyrrolo[2,3-b]pyridin-3-yl)-phenyl]-acetamide. LC-MS (ES) m/e 328 [M+H]⁺.

### Example 33

4-(5-phenyl-1H-pyrrolo[2,3-b]pyridin-3-yl)-phenylamine. LC-MS (ES) m/e 286 [M+H]⁺.

### Example 34

4-(5-phenyl-1H-pyrrolo[2,3-b]pyridin-3-yl)-phenol. LC-MS (ES) m/e 287 [M+H]⁺.

### Example 35

4-(5-phenyl-1H-pyrrolo[2,3-b]pyridin-3-yl)-benzylamine. LC-MS (ES) m/e 300 [M+H]⁺.

### Example 36

3-(5-phenyl-1H-pyrrolo[2,3-b]pyridin-3-yl)-phenol. LC-MS (ES) m/e 287 [M+H]⁺.

### Example 37

5-(3,4-dimethoxyphenyl)-3-pyridin-4-yl-1H-pyrrolo[2,3-b]pyridine. LC-MS (ES) m/e 332 [M+H]⁺.

### Example 38

4-[5-(3,4-dimethoxyphenyl)-1H-pyrrolo[2,3-b]pyridin-yl]-phenol. LC-MS (ES) m/e 347 [M+H]⁺.

### Example 39

4-[5-(3,4-dimethoxyphenyl)-1H-pyrrolo[2,3-b]pyridin-yl]-phenylamine. LC-MS (ES) m/e 346 [M+H]⁺.

### Example 40

4-[5-(3,4-dimethoxyphenyl)-1H-pyrrolo[2,3-b]pyridin-3-yl]-benzoic acid. LC-MS (ES) m/e 375 [M+H]⁺.

### Example 41

4-[5-(4-chlorophenyl)-1H-pyrrolo[2,3-b]pyridin-3-yl]-benzoic acid. LC-MS (ES) m/e 349 [M+H]⁺.

### Example 42

N-[4-(5-phenyl-1H-pyrrolo[2,3-b]pyridin-3-yl)-phenyl]-acetamide. LC-MS (ES) m/e 328 [M+H]⁺.

### Example 43

3,5-bis-(4-hydroxyphenyl)-1H-pyrrolo[2,3-b]pyridine. LC-MS (ES) m/e 303 [M+H]⁺.

### Example 44

3,5-bis-(4-carboxyphenyl)-1H-pyrrolo[2,3-b]pyridine. LC-MS (ES) m/e 359 [M+H]⁺.

### Example 45

4-[5-(4-aminophenyl)-1H-pyrrolo[2,3-b]pyridin-3-yl)-benzylamine. LC-MS (ES) m/e 329 [M+H]⁺.

### Example 46

4-[5-(4-aminophenyl)-1H-pyrrolo[2,3-b]pyridin-3-yl)-benzoic acid. LC-MS (ES) m/e 344 [M+H]⁺.

### Example 47

4-[5-(2-fluorobiphen-4-yl)-1H-pyrrolo[2,3-b]pyridin-3-yl)-benzoic acid LC-MS (ES) m/e 409 [M+H]⁺.

### Example 48

N-[3-(5-thiophen-3-yl-1H-pyrrolo[2,3-b]pyridin-3-yl)-phenyl]-acetamide. LC-MS (ES) m/e 334 [M+H]⁺.

### Example 49

4-(5-thiophen-3-yl-1H-pyrrolo[2,3-b]pyridin-3-yl)-benzoic acid. LC-MS (ES) m/e 321 [M+H]⁺.

### Example 50

4-(5-thiophen-3-yl-1H-pyrrolo[2,3-b]pyridin-3-yl)-phenol. LC-MS (ES) m/e 293 [M+H]⁺.

### Example 51

4-(5-thiophen-3-yl-1H-pyrrolo[2,3-b]pyridin-3-yl)-benzamide. LC-MS (ES) m/e 320 [M+H]⁺.

### Example 52

N-[3-(5-pyridin-3-yl-1H-pyrrolo[2,3-b]pyridin-3-yl)-phenyl]-acetamide. LC-MS (ES) m/e 329 [M+H]⁺.

### Example 53

4-(5-pyridin-3-yl-1H-pyrrolo[2,3-b]pyridin-3-yl)-benzoic acid. LC-MS (ES) m/e 316 [M+H]⁺.

### Example 54

4-(5-pyridin-3-yl-1H-pyrrolo[2,3-b]pyridin-3-yl)-phenol. LC-MS (ES) m/e 288 [M+H]⁺.

### Example 55

4-(5-thiophen-3-yl-1H-pyrrolo[2,3-b]pyridin-3-yl)-benzylamine. LC-MS (ES) m/e 306 [M+H]⁺.

### Example 56

3-(1H-indol-5-yl)-5-thiophen-3-yl-1H-pyrrolo[2,3-b]pyridine. LCMS (ES) m/e 316 [M+H]⁺.

### Example 57

N-[4-(5-thiophen-3-yl-1H-pyrrolo[2,3b]pyridin-3-yl)-phenyl]-acetamide. LC-MS (ES) m/e 334 [M+H]⁺.

### Example 58

5-pyridin-3-yl-3-pyridin-4-yl-1H-pyrrolo[2,3-b]pyridine. LC-MS (ES) m/e 273 [M+H]⁺.

### Example 59

4-(5-pyridin-3-yl-1H-pyrrolo[2,3-b]pyridin-3-yl)-benzamide. LC-MS (ES) m/e 315 [M+H]⁺.

### Example 60

4-[3-(2-fluorobiphenyl-4-yl-1H-pyrrolo[2,3-b]pyridin-5-yl]-benzylamine. LC-MS (ES) m/e 394 [M+H]⁺.

### Example 61

4-(5-pyridin-3-yl-1H-pyrrolo[2,3-b]pyridin-3-yl)-phenylamine. LC-MS (ES) m/e 287 [M+H]⁺.

### Example 62

{3-[5-(4-methanesulfonylphenyl-1H-pyrrolo[2,3-b]pyridin-3-yl]-phenyl}-acetic acid. LC-MS (ES) m/e 407 [M+H]⁺.

### Example 63

N-[3-(3-thiophen-3-yl-1H-pyrrolo[2,3-b]pyridin-5-yl)-phenyl]-acetamide. LC-MS (ES) m/e 334 [M+H]⁺.

### Example 64

N-{3-[3-(3-pyridinyl)-1H-pyrrolo[2,3-b]pyridine-5-yl]phenyl}acetamide-3-yl-1H-pyrrolo[2,3-b]pyridin-5-yl)-phenyl]-acetamide. LC-MS (ES) m/e 329 [M+H]⁺.

### Example 65

4-[5-(3-acetylaminophenyl)-1H-pyrrolo[2,3-b]pyridin-3-yl]-benzoic acid. LC-MS (ES) m/e 372 [M+H]⁺.

### Example 66

N-{3-[3-(2,3-difluorophenyl)-1H-pyrrolo[2,3-b]pyridin-5-yl]-phenyl}-acetamide. LC-MS (ES) m/e 364 [M+H]⁺.

### Example 67

N-{3-[3-(4-hydroxyphenyl)-1H-pyrrolo[2,3-b]pyridin-5-yl]-phenyl}-acetamide. LC-MS (ES) m/e 344 [M+H]⁺.

### Example 68

N-{3-[3-(4-aminomethylphenyl)-1-pyrrolo[2,3-b]pyridin-5-yl]-phenyl}-acetamide. LC-MS (ES) m/e 357 [M+H]⁺.

### Example 69

N-{3-[3-(4-aminophenyl)-1H-pyrrolo[2,3-b]pyridin-5-yl]-phenyl}-acetamide. LC-MS (ES) m/e 343 [M+H]⁺.

### Example 70

N-{3-[3-(1H-indol-5-yl)-1H-pyrrolo[2,3-b]pyridin-5-yl]-phenyl}-acetamide. LC-MS (ES) m/e 367 [M+H]⁺.

### Example 71

4-[3-(2-fluorobiphenyl-4-yl)-1H-pyrrolo[2,3-b]pyridin-5-yl]-benzoic acid. LC-MS (ES) m/e 409 [M+H]⁺.

### Example 72

N-{3-[3-(4-pyridinyl)-1H-pyrrolo[2,3-b]pyridin-5-yl]phenyl}acetamide

### Example 73

N-{3-[5-(3-fluorophenyl)-1H-pyrrolo[2,3-b]pyridin-3-yl]-phenyl}-acetamide. LC-MS (ES) m/e 346 [M+H]⁺.

### Example 74

4-[5-(3-acetylaminophenyl)-1H-pyrrolo[2,3-b]pyridin-3-yl]-benzamide. LC-MS (ES) m/e 371 [M+H]⁺.

### Example 75

4-[5-(3-fluorophenyl)-1H-pyrrolo[2,3-b]pyridin-3-yl]-benzoic acid. LC-MS (ES) m/e 333 [M+H]⁺.

### Example 76

4-[5-(3-fluorophenyl)-1H-pyrrolo[2,3-b]pyridin-3-yl]-phenylamine. LC-MS (ES) m/e 304 [M+H]⁺.

### Example 77

N-{4-[5-(3-fluorophenyl)-1H-pyrrolo[2,3-b]pyridin-3-yl]-phenyl}-acetamide. LC-MS (ES) m/e 346 [M+H]⁺.

### Example 78

4-[5-(3-fluorophenyl)-1H-pyrrolo[2,3-b]pyridin-3-yl]-benzamide. LC-MS (ES) m/e 332 [M+H]⁺.

### Example 79

2-chloro-[4-(5-phenyl-1H-pyrrolo[2,3-b]pyridin-3-yl)-phenyl]-benzamide. LC-MS (ES) m/e 424 [M+H]⁺.

### Example 80

2-phenyl-N-[4-(5-phenyl-1H-pyrrolo[2,3-b]pyridin-3-yl)-phenyl]-acetamide. LC-MS (ES) m/e 404 [M+H]⁺.

### Example 81

2-chloro-N-[3-(5-phenyl-1H-pyrrolo[2,3-b]pyridin-3-yl)-benzyl]-benzamide. LC-MS (ES) m/e 438 [M+H]⁺.

### Example 82

2-phenyl-N-[3-(5-phenyl-1H-pyrrolo[2,3-b]pyridin-3-yl)-benzyl]-acetamide. LC-MS (ES) m/e 418 [M+H]⁺.

### Example 83

(4-{5-[3-(methyloxy)phenyl]-1H-pyrrolo[2,3-b]pyridin-3-yl}phenyl)acetic acid. LC-MS (ES) m/e 359.2 [M+H]⁺.

### Example 84

3-[4-(5-{4-[(methylsulfonyl)amino]phenyl}-1H-pyrrolo[2,3-b]pyridin-3-yl)phenyl]propanoic acid. LC-MS (ES) m/e 436.4[M+H]⁺.

### Example 85

[4-(5-{4-[(methylsulfonyl)amino]phenyl}-1H-pyrrolo[2,3-b]pyridin-3-yl)phenyl]acetic acid. LC-MS (ES) m/e 422.4 [M+H]⁺.

### Example 86

(4-{5-[3,4,5-tris(methyloxy)phenyl]-1H-pyrrolo[2,3-b]pyridin-3-yl}phenyl)acetic acid. LC-MS (ES) m/e 419.2 [M+H]⁺.

### Example 87

3-(4-{5-[3,4,5-tris(methyloxy)phenyl]-1H-pyrrolo[2,3-b]pyridin-3-yl}phenyl)propanoic acid. LC-MS (ES) m/e 433.2 [M+H]⁺.

### Example 88

{4-[5-(6-quinolinyl)-1H-pyrrolo[2,3-b]pyridin-3-yl]phenyl}acetic acid. LC-MS (ES) m/e 380.4 [M+H]⁺.

### Example 89

3-{4-[5-(6-quinolinyl)-1H-pyrrolo[2,3-b]pyridin-3-yl]phenyl}propanoic acid. LC-MS (ES) m/e 394.0 [M+H]⁺.

### Example 90

{4-[5-(3-quinolinyl)-1H-pyrrolo[2,3-b]pyridin-3-yl]phenyl}acetic acid. LC-MS (ES) m/e 380.4 [M+H]⁺.

### Example 91

(4-[5-(5-quinolinyl)-1H-pyrrolo[2,3-b]pyridin-3-yl]phenylacetic acid. LC-MS (ES) m/e 380.4 [M+H]⁺.

### Example 92

3-{4-[5-(5-quinolinyl)-1H-pyrrolo[2,3-b]pyridin-3-yl]phenyl}propanoic acid. LC-MS (ES) m/e 394.2 [M+H]⁺.

### Example 93

3-(4-{5-[6-(methyloxy)-2-naphthalenyl]-1H-pyrrolo[2,3-b]pyridin-3-yl}phenyl)propanoic acid. LC-MS (ES) m/e 423.2 [M+H]⁺.

### Example 94

3-{4-[5-(3,4-dimethylphenyl)-1H-pyrrolo[2,3-b]pyridin-3-yl]phenyl}propanoic acid. LC-MS (ES) m/e 371.4 [M+H]⁺.

### Example 95

{4-[5-(3,4-dimethylphenyl)-1H-pyrrolo[2,3-b]pyridin-3-yl]phenyl}acetic acid. LC-MS (ES) m/e 357.4 [M+H]⁺.

### Example 96

{4-[5-(2,3-dimethylphenyl)-1H-pyrrolo[2,3-b]pyridin-3-yl]phenyl}acetic acid. LC-MS (ES) m/e 357.2 [M+H]⁺.

### Example 97

3-{4-[5-(2,3-dimethylphenyl)-1H-pyrrolo[2,3-b]pyridin-3-yl]phenyl}propanoic acid. LC-MS (ES) m/e 371.4 [M+H]⁺.

### Example 98

{4-[5-(2,3-dichlorophenyl)-1H-pyrrolo[2,3-b]pyridin-3-yl]phenyl}acetic acid. LC-MS (ES) m/e 399.2 [M+H]⁺.

### Example 99

3-{4-[5-(2,3-dichlorophenyl)-1H-pyrrolo[2,3-b]pyridin-3-yl]phenyl}propanoic acid. LC-MS (ES) m/e 411.2 [M+H]⁺.

### Example 100

{4-[5-(1-benzothien-3-yl)-1H-pyrrolo[2,3-b]pyridin-3-yl]phenyl}acetic acid. LC-MS (ES) m/e 385.2 [M+H]⁺.

### Example 101

[(3-{5-[3-(methyloxy)phenyl]-1H-pyrrolo[2,3-b]pyridin-3-yl}phenyl)methyl]amine. MS m/e 330.4 [M+H]⁺.

### Example 102

7-[5-(2-naphthalenyl)-1H-pyrrolo[2,3-b]pyridin-3-yl]-1,2,3,4-tetrahydroisoquinoline. MS m/e 376.4 [M+H]⁺.

### Example 103

2-fluoro-4-(5-(2-naphthalenyl)-1H-pyrrolo[2,3-b]pyridin-3-yl]benzoic acid. MS m/e 379.4 [M+H]⁺.

### Example 104

2-fluoro-4-{5-[3-(methyloxy)phenyl]-1H-pyrrolo[2,3-b]pyridin-3-yl}benzoic acid. MS m/e 359.2 [M+H]⁺.

### Example 105

2-methyl-4-{5-[3-(methyloxy)phenyl]-1H-pyrrolo[2,3-b]pyridin-3-yl}benzoic acid. MS m/e 359 [M+H]⁺.

### Example 106

5-[5-(2-naphthalenyl)-1H-pyrrolo[2,3-b]pyridin-3-yl]-2-thiophenecarbaldehyde. MS m/e 355.4 [M+H]⁺.

### Example 107

5-{5-[3-(methyloxy)phenyl]-1H-pyrrolo[2,3-b]pyridin-3-yl}-2-thiophenecarbaldehyde. MS m/e 335.2 [M+H]⁺.

### Example 108

2-methyl-4-[5-(2-naphthalenyl)-1H-pyrrolo[2,3-b]pyridin-3-yl]benzoic acid. MS m/e 379.4 [M+H]⁺.

### Example 109

(4-{5-[6-(methyloxy)-2-naphthalenyl]-1H-pyrrolo[2,3-b]pyridin-3-yl}phenyl)acetic acid. MS m/e 409.4 [M+H]⁺.

### Example 110

2-fluoro-4-{5-[6-(methyloxy)-2-naphthalenyl]-1H-pyrrolo[2,3-b]pyridin-3-yl}benzoic acid. MS m/e 413.4 [M+H]⁺.

### Example 111

2-fluoro-4-[5-(5-quinolinyl)-1H-pyrrolo[2,3-b]pyridin-3-yl]benzoic acid. MS m/e 384.4 [M+H]⁺.

### Example 112

4-[5-(5-quinolinyl)-1H-pyrrolo[2,3-b]pyridin-3-yl]benzoic acid. MS m/e 366.2 [M+H]⁺.

### Example 113

2-methyl-4-[5-(5-quinolinyl)-1H-pyrrolo[2,3-b]pyridin-3-yl]benzoic acid. MS m/e 380.4 [M+H]⁺.

### Example 114

4-[5-(3,4-dimethylphenyl)-1H-pyrrolo[2,3-b]pyridin-3-yl]benzoic acid. MS m/e 343.2 [M+H]⁺.

### Example 115

4-[5-(3,4-dimethylphenyl)-1H-pyrrolo[2,3-b]pyridin-3-yl]-2-fluorobenzoic acid. MS m/e 361.4 [M+H]⁺.

### Example 116

4-[5-(3,4-dimethylphenyl)-1H-pyrrolo[2,3-b]pyridin-3-yl]-2-methylbenzoic acid. MS m/e 357.2 [M+H]⁺.

### Example 117

4-[5-(2,3-dichlorophenyl)-1H-pyrrolo[2,3-b]pyridin-3-yl]benzoic acid. MS m/e 383 [M+H]⁺.

### Example 118

4-[5-(2,3-dichlorophenyl)-1H-pyrrolo[2,3-b]pyridin-3-yl]-2-methylbenzoic acid. MS m/e 397 [M+H]⁺.

### Example 119

4-[5-(1-benzothien-3-yl)-1H-pyrrolo[2,3-b]pyridin-3-yl]benzoic acid. MS nm/e 371.2 [M+H]⁺.

### Example 120

4-[5-(1-benzothien-3-yl)-1H-pyrrolo[2,3-b]pyridin-3-yl]-2-fluorobenzoic acid. MS m/e 389.2 [M+H]⁺.

### Example 121

4-[5-(1-benzothien-3-yl)-1H-pyrrolo[2,3-b]pyridin-3-yl]-2-methylbenzoic acid. MS m/e 385.2 [M+H]⁺.

### Example 122

6-{3-[4-(ethylsulfonyl)phenyl]-1H-pyrrolo[2,3-b]pyridin-5-yl}quinoline. MS m/e 414 [M+H]⁺.

### Example 123

4-(5-{3-[(methylsulfonyl)amino]phenyl}-1H-pyrrolo[2,3-b]pyridin-3-yl)benzoic acid. MS m/e 408.4 [M+H]⁺.

### Example 124

3-[4-(butyloxy)phenyl]-5-[3-(methylsulfonyl)phenyl]-1H-pyrrolo[2,3-b]pyridine. MS m/e 421.2 [M+H]⁺.

### Example 125

N-(3-{3-[4-(aminomethyl)phenyl]-1H-pyrrolo[2,3-b]pyridin-5-yl}phenyl)methanesulfonamide. MS m/e 393.0 [M+H]⁺.

### Example 126

N-(3-{3-[3-(aminomethyl)phenyl]-1H-pyrrolo[2,3-b]pyridin-5-yl}phenyl)methanesulfonamide. MS m/e 393.2 [M+H]⁺.

### Example 127

3-amino-5-(5-{3-[(methylsulfonyl)amino]phenyl}-1H-pyrrolo[2,3-b]pyridin-3-yl)benzoic acid. MS m/e 423.2 [M+H]⁺.

### Example 128

2-fluoro-4-(5-{3-[(methylsulfonyl)amino]phenyl}-1H-pyrrolo[2,3-b]pyridin-3-yl}benzoic acid. MS m/e 426.2 [M+H]⁺.

### Example 129

3-amino-5-{5-[3-(methylsulfonyl)phenyl]-1H-pyrrolo[2,3-b]pyridin-3-yl}benzoic acid. MS m/e 408.4 [M+H]⁺.

### Example 130

2-fluoro-4-{5-[3-(methylsulfonyl)phenyl]-1H-pyrrolo[2,3-b]pyridin-3-yl}benzoic acid. MS m/e 411.4 [M+H]⁺.

### Example 131

[(4-{5-[3-(methylsulfonyl)phenyl]-1H-pyrrolo[2,3-b]pyridin-3-yl}phenyl)methyl]amine. MS m/e 378 [M+H]⁺.

### Example 132

[(3-{5-[3-(methylsulfonyl)phenyl]-1H-pyrrolo[2,3-b]pyridin-3-yl}phenyl)methyl]amine. MS m/e 378.4 [M+H]⁺.

### Example 133

5-{5-[3-(methylsulfonyl)phenyl]-1H-pyrrolo[2,3-b]pyridin-3-yl}-2-thiophenecarbaldehyde. MS m/e 383.2 [M+H]⁺.

### Example 134

4-{5-[3-(methylsulfonyl)phenyl]-1H-pyrrolo[2,3-b]pyridin-3-yl}benzoic acid. MS m/e 393.2 [M+H]⁺.

### Example 135

N-(4-{3-[4-(butyloxy)phenyl]-1H-pyrrolo[2,3-b]pyridin-5-yl}phenyl)methanesulfonamide. MS m/e 436.4 [M+H]⁺.

### Example 136

1,1-dimethylethyl[2-(3-{5-[3-(methylsulfonyl)phenyl]-1H-pyrrolo[2,3-b]pyridin-3-yl}phenyl)ethyl]carbamate. MS m/e 492.6 [M+H]⁺.

### Example 137

3-amino-5-(5-{4-[(methylsulfonyl)amino]phenyl}-1H-pyrrolo[2,3-b]pyridin-3-yl)benzoic acid. MS m/e 422.8 [M+H]⁺.

### Example 138

2-fluoro-4-(5-{4-[(methylsulfonyl)amino]phenyl}-1H-pyrrolo[2,3-b]pyridin-3-yl)benzoic acid. MS m/e 426.2 [M+H]⁺.

### Example 139

4-(5-{4-[(methylsulfonyl)amino]phenyl}-1H-pyrrolo[2,3-b]pyridin-3-yl)benzoic acid. MS m/e 408.4 [M+H]⁺.

### Example 140

N-(4-{3-[4-(aminomethyl)phenyl]-1H-pyrrolo[2,3-b]pyridin-5-yl}phenyl)methanesulfonamide. MS m/e 393.2 [M+H]⁺.

### Example 141

N-(4-{3-[3-(aminomethyl)phenyl]-1H-pyrrolo[2,3-b]pyridin-5-yl}phenyl)methanesulfonamide. MS m/e 392.8 [M+H]⁺.

### Example 142

N-{4-[3-(5-formyl-2-thienyl)-1H-pyrrolo[2,3-b]pyridin-5-yl]phenyl}methanesulfonamide. MS m/e 398.0 [M+H]⁺.

### Example 143

7-(5-[3-(methylsulfonyl)phenyl]-1H-pyrrolo[2,3-b]pyridin-3-yl)-1,2,3,4-tetrahydroisoquinoline. MS m/e 404.4 [M+H]⁺.

### Example 144

N-{3-[3-(1,2,3,4-tetrahydro-7-isoquinolinyl)-1H-pyrrolo[2,3-b]pyridin-5-yl]phenyl}methanesulfonamide. MS m/e 419.2 [M+H]⁺.

### Example 145

N-{4-[3-(1,2,3,4-tetrahydro-7-isoquinolinyl)-1H-pyrrolo[2,3-b]pyridin-5-yl]phenyl}methanesulfonamide. MS m/e 419.2 [M+H]⁺.

### Example 146

2-methyl-4-{5-[3-(methylsulfonyl)phenyl]-1H-pyrrolo[2,3-b]pyridin-3-yl}benzoic acid. MS m/e 407.2 [M+H]⁺.

### Example 147

5-{5-[3-(methylsulfonyl)phenyl]-1H-pyrrolo[2,3-b]pyridin-3-yl}-2-thiophenecarboxylic acid. MS m/e 399 [M+H]⁺.

### Example 148

3-{3-[3-(aminomethyl)phenyl]-1H-pyrrolo[2,3-b]pyridin-5-yl}benzonitrile. MS m/e 325.4 [M+H]⁺.

### Example 149

2-fluoro-4-{5-[6-(methyloxy)-3-pyridinyl]-1H-pyrrolo[2,3-b]pyridin-3-yl}benzoic acid. MS m/e 364 [M+H]⁺.

### Example 150

3-[3-(1,2,3,4-tetrahydro-7-isoquinolinyl)-1H-pyrrolo[2,3-b]pyridin-5-yl]benzonitrile. MS m/e 351.4 [M+H]⁺.

### Example 151

7-{5-[3,4,5-tris(methyloxy)phenyl]-1H-pyrrolo[2,3-b]pyridin-3-yl}-1,2,3,4-tetrahydroisoquinoline. MS m/e 416.2 [M+H]⁺.

### Example 152

4-{5-[3,4,5-tris(methyloxy)phenyl]-1H-pyrrolo[2,3-b]pyridin-3-yl}benzoic acid. MS m/e 405.6 [M+H]⁺.

### Example 153

2-fluoro-4-{5-[3,4,5-tris(methyloxy)phenyl]-1H-pyrrolo[2,3-b]pyridin-3-yl}benzoic acid. MS m/e 423.2 [M+H]⁺.

### Example 154

2-amino-4-{5-[3,4,5-tris(methyloxy)phenyl]-1H-pyrrolo[2,3-b]pyridin-3-yl}benzoic acid. MS m/e 420.2 [M+H]⁺.

### Example 155

4-[5-(6-quinolinyl)-1H-pyrrolo[2,3-b]pyridin-3-yl]benzoic acid. MS m/e 366.2 [M+H]⁺.

### Example 156

4-[5-(3-cyanophenyl)-1H-pyrrolo[2,3-b]pyridin-3-yl]-2-fluorobenzoic acid. MS m/e 358.2 [M+H]⁺.

### Example 157

4-[5-(2,3-dimethylphenyl)-1H-pyrrolo[2,3-b]pyridin-3-yl]benzoic acid. MS m/e 343.2 [M+H]⁺.

### Example 158

4-[5-(2,3-dimethylphenyl)-1H-pyrrolo[2,3-b]pyridin-3-yl]-2-fluorobenzoic acid. MS m/e 361.2 [M+H]⁺.

### Example 159

2-methyl-4-[5-(6-quinolinyl)-1H-pyrrolo[2,3-b]pyridin-3-yl]benzoic acid. MS m/e 380.2 [M+H]⁺.

### Example 160

2-methyl-4-[5-(1-naphthalenyl)-1H-pyrrolo[2,3-b]pyridin-3-yl]benzoic acid. MS m/e 379.4 [M+H]⁺.

### Example 161

4-[5-(3-cyanophenyl)-1H-pyrrolo[2,3-b]pyridin-3-yl]-2-methylbenzoic acid. MS m/e 354.2 [M+H]⁺.

### Example 162

2-methyl-4-{5-[6-(methyloxy)-3-pyridinyl]-1H-pyrrolo[2,3-b]pyridin-3-yl}benzoic acid. MS m/e 360.2 [M+H]⁺.

### Example 163

2-methyl-4-{5-[3,4,5-tris(methyloxy)phenyl]-1H-pyrrolo[2,3-b]pyridin-3-yl}benzoic acid. MS m/e 419.2 [M+H]⁺.

### Example 164

2-(1-methylethyl)-4-[5-(1-naphthalenyl)-1H-pyrrolo[2,3-b]pyridin-3-yl]benzoic acid. MS m/e 407.4 [M+H]⁺.

### Example 165

4-[5-(3-cyanophenyl)-1H-pyrrolo[2,3-b]pyridin-3-yl]-2-(1-methylethyl)benzoic acid. MS m/e 382.2 [M+H]⁺.

### Example 166

2-(1-methylethyl)-4-[5-(6-quinolinyl)-1H-pyrrolo[2,3-b]pyridin-3-yl]benzoic acid. MS m/e 408.2 [M+H]⁺.

### Example 167

4-[5-(2,3-dimethylphenyl)-1H-pyrrolo[2,3-b]pyridin-3-yl]-2-methylbenzoic acid. MS m/e 357.4 [M+H]⁺.

### Example 168

2-chloro-4-{5-[3-(methylsulfonyl)phenyl]-1-H-pyrrolo-[2,3-b]pyridin-3-yl}benzoic acid. MS m/e 427 [M+H]⁺.

### Example 169

4-{5-[3-(methylsulfonyl)phenyl]-1-H-pyrrolo-[2,3-b]pyridin-3-yl}-2,6-bis(trifluoromethyl)benzoic acid. MS m/e 529 [M+H]⁺.

### Example 170

methyl 2-(azidomethyl)-4-(5-phenyl-1H-pyrrolo[2,3-b]pyridin-3-yl)benzoate. MS m/e 384 [M+H]⁺.

### Example 171

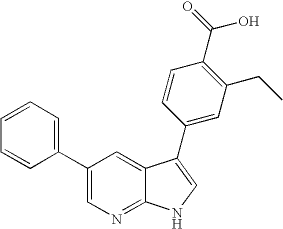

### Preparation of 2-ethyl-4-(5-phenyl-1H-pyrrolo[2,3-b]pyridin-3-yl)benzoic acid

### a) 1,1-dimethylethyl 3-bromo-5-phenyl-1H-pyrrolo[2,3-b]pyridine-1-carboxylate

To a suspension of 3-bromo-5-phenyl-1H-pyrrolo[2,3-b]pyridine hydrobromide (5.16 g, 14.6 mmol) in CH₂Cl₂ (50 mL) was added diisopropylethylamine (7.63 mL, 43.8 mmol) slowly. A dark orange solution resulted, and di-tert-butyldicarbonate (3.83 g, 17.5 mmol) and a spatula tip of dimethylaminopyridine was added. The reaction was stirred for five hours, and then extracted twice with water. The organic layer was dried over Na₂SO₄, filtered, and concentrated. The crude product was purified using flash silica chromatography (1-7% ethyl acetate/hexanes) to give the title compound as a white solid (4.05 g, 74%). ¹H NMR (400 MHz, MeOD) δ 8.68 (d, J=2.4 Hz, 1H), 8.09 (d, J=2.0 Hz, 1H), 7.90 (s, 1H), 7.70 (m, 2H), 7.53 (t, J=7.6 Hz, 2H), 7.44 (m, 1H), 1.72 (6, 9H). LCMS m/e 373.0 [M]⁺.

### b) 2-ethyl-4-(5-phenyl-1H-pyrrolo[2,3-b]pyridin-3-yl)benzoic acid

To a solution of 1,1-dimethylethyl 3-bromo-5-phenyl-1H-pyrrolo[2,3-b]pyridine-1-carboxylate (0.192 g, 0.514 mmol) in dioxane:water (2.5:1, 5 mL) was added 4-(dihydroxyboranyl)-2-ethylbenzoic acid (0.120 g, 0.616 mmol), potassium carbonate (0.21 g, 1.54 mmol), and [1,1′-bis(diphenylphosphino)ferrocene]dichloropalladium(II) complex with dichloromethane (0.021 g, 0.026 mmol). The mixture was heated using a microwave for five minutes at 100° C. to produce the Suzuki product and then for five minutes at 150° C. to thermally deprotect the indole nitrogen. The reaction was diluted with ethyl acetate (10 mL) and 10 mL of 1N HCl and filtered through celite. The filter cake was washed with ethyl acetate:water and the filtrate layers were separated. The aqueous layer was extracted twice with ethyl acetate (10 mL). The combined organic extracts were dried over Na₂SO₄, filtered, and concentrated. The crude material was purified using flash silica chromatography (1-10% MeOH/CH₂Cl₂). The product fractions were concentrated, and the crude solid was triturated in 1% MeOH/CH₂Cl₂. The suspension was filtered to give the title compound as a tan powder (0.0585 g, 33%). ¹H NMR (400 MHz, DMSO-d₆) δ 8.59 (d, J=2.0 Hz, 1H), 8.48 (d, J=2.0 Hz, 1H), 8.08 (d, J=2.4 Hz, 1H), 7.91 (d, J=8.0 Hz, 1H), 7.76 (m, 4H), 7.52 (t, J=8.0 Hz, 2H), 7.40 (t, J=7.2 Hz, 1H), 3.05 (q, J=7.2 Hz, 2H), 1.24 (t, J=7.6 Hz, 3H). LCMS m/e 343.2 [M+H]⁺.

### Examples 172-181

Compounds of Examples 172-181 were prepared following the same general procedure described above for Example 171.

### Example 172

2-(methylamino)-4-(5-phenyl-1H-pyrrolo[2,3-b]pyridin-3-yl)benzoic acid. MS m/e 344.0 [M+H]⁺.

### Example 173

2-(dimethylamino)-4-(5-phenyl-1H-pyrrolo[2,3-b]pyridin-3-yl)benzoic acid. MS m/e 358.4 [M+H]⁺.

### Example 174

2-cyclopentyl-4-(5-phenyl-1H-pyrrolo[2,3-b]pyridin-3-yl)benzoic acid. MS m/e 383.0 [M+H]⁺.

### Example 175

4-(5-phenyl-1H-pyrrolo[2,3-b]pyridin-3-yl)-2-propylbenzoic acid. MS m/e 357.2 [M+H]⁺.

### Example 176

2,6-difluoro-4-(5-phenyl-1H-pyrrolo[2,3-b]pyridin-3-yl)benzoic acid. MS m/e 351.4 [M+H]⁺.

### Example 177

2,6-dimethyl-4-(5-phenyl-1H-pyrrolo[2,3-b]pyridin-3-yl)benzoic acid. MS m/e 343.2 [M+H]⁺.

### Example 178

2-(2-propyl)-4-(5-phenyl-1H-pyrrolo[2,3b]pyridin-3-yl)benzoic acid. MS m/e 357.0 [M+H]⁺.

### Example 179

6-(5-phenyl-1H-pyrrolo[2,3-b]pyridin-3-yl)-1H-indazole. MS m/e 311.2 [M+H]⁺.

### Example 180

2-(2-methylpropyl)-4-(5-phenyl-1H-pyrrolo[2,3-b]pyridin-3-yl)benzoic acid. MS m/e 371.4 [M+H]⁺.

### Example 181

2-methyl-4-(5-phenyl-1H-pyrrolo[2,3-b]pyridin-3-yl)benzoic acid. MS m/e 329 [M+H]⁺.

### Example 182

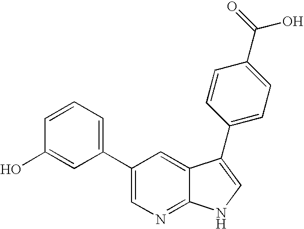

### Preparation of 4-[5-(3-hydroxyphenyl)-1-H-pyrrolo[2,3-b]pyridin-3-yl]benzoic acid

### a) 2,2,2-trifluoroacetic acid 3-(3-bromo-1H-pyrrolo[2,3-b]pyridin-5-yl)-phenyl ester

A solution of 3-(1H)-pyrrolo[2,3-b]pyridin-5-yl-phenol (1.31 g, 6.21 mmol) in chloroform (20 mL) was cooled to 0° C. A solution of trifluoroacetic anhydride (1.29 mL, 9.31 mmol) in chloroform (10 mL) was added dropwise to the reaction mixture. The reaction mixture was stirred at room temperature for 30 minutes, and then diluted with water (50 mL) and extracted with chloroform (2 50-mL portions). The combined organics were dried over anhydrous sodium sulfate and were concentrated to provide 2,2,2-trifluoroacetic acid 3-(3-bromo-1H-pyrrolo[2,3-b]pyridin-5-yl)-phenyl ester, which was used in the next reaction without further purification. ¹H NMR (400 MHz, CDCl₃) δ 8.45 (s, 1H), 8.23 (s, 1H), 7.62 (m, 2H), 7.48 (m, 2H), 7.32 (m, 1H). TLC (50% ethyl acetate in hexanes) Rf=0.45.

### b) 4-[5-(3-hydroxyphenyl)-1-H-pyrrolo[2,3-b]pyridin-3-yl]benzoic acid

The title compound was prepared from 2,2,2-trifluoroacetic acid 3-(3-bromo-1H pyrrolo[2,3-b]pyridin-5-yl)-phenyl ester using the procedure described in Example 1. ¹H NMR (400 MHz, DMSO-d₆) δ 12.2 (s, 1H), 9.56 (s, 1H), 8.53 (d, J=2 Hz, 1H), 8.46 (d, J=2 Hz, 1H), 8.11 (d, J=2.8 Hz, 1H), 8.02 (d, J=8.8 Hz, 2H), 7.94 (d, J=8.8 Hz, 2H), 7.30 (t, J=8.4 Hz, 1H), 7.19 (d, J=7.6 Hz, 1H), 7.13 (s, 1H), 6.79 (dd, J=8, 2.4 Hz, 1H). MS m/e 331.4 [M+H]⁺.

### Examples 183-197

Compounds of Examples 183-197 were prepared following the same general procedure described above for Example 182.

### Example 183

3-amino-5-[5-(3-hydroxyphenyl)1H-pyrrolo[2,3-b]-pyridin-3-yl]-benzoic acid. MS m/e 346.2 [M+H]⁺.

### Example 184

{4-[5-hydroxyphenyl)-1H-pyrrolo[2,3-b]pyridin-3-yl]-phenyl}acetic acid. MS m/e 345.2 [M+H]⁺.

### Example 185

4-[5-(3-aminophenyl)-1-H-pyrrolo[2,3-b]pyridin-3-yl]benzoic acid. MS m/e 330.4 [M+H]⁺.

### Example 186

3-(4-[5-hydroxyphenyl)-1H-pyrrolo[2,3-b]pyridin-3-yl]-phenyl)-propionic acid. MS m/e 359.2 [M+H]⁺.

### Example 187

3-{4-[5-(3-aminophenyl)-1H-pyrrolo[2,3-b]pyridin-3-yl]phenyl}propanoic acid. MS m/e 358.2 [M+H]⁺.

### Example 188

{4-[5-(3-aminophenyl)-1H-pyrrolo[2,3-b]pyridin-3-yl]phenyl}acetic acid. MS m/e 344.0 [M+H]⁺.

### Example 189

4-{5-[3-(aminomethyl)phenyl]-1H-pyrrolo[2,3-b]pyridin-3-yl}benzoic acid. MS m/e 344.2 [M+H]⁺.

### Example 190

(4-{5-[3-(aminomethyl)phenyl]-1H-pyrrolo[2,3-b]pyridin-3-yl}phenyl)acetic acid. MS m/e 372.2 [M+H]⁺.

### Example 191

4-[5-(3-hydroxyphenyl)-1-H-pyrrolo-[2,3-b]pyridin-3-yl]benzoic acid. MS m/e 331 [M+H]⁺.

### Example 192

2-fluoro-4-[5-(3-hydroxyphenyl)-1-H-pyrrolo-[2,3-b]pyridin-3-yl]benzoic acid. MS m/e 349 [M+H]⁺.

### Example 193

5-[5-(3-hydroxyphenyl)-1-H-pyrrolo-[2,3-b]pyridin-3-yl]thiophene-2-carbaldehyde. MS m/e 321 [M+H]⁺.

### Example 194

3-{3-[3-(2-aminoethyl)phenyl]-1-H-pyrrolo-[2,3-b]pyridin-5-yl}phenol. MS m/e 330 [M+H]⁺.

### Example 195

3-[3-(1,2,3,4-tetrahydroisoquinolin-7-yl)-1-H-pyrrolo-[2,3-b]pyridin-5-yl]phenol. MS m/e 342 [M+H]⁺.

### Example 196

3-amino-5-[5-(3-aminophenyl)-1-H-pyrrolo-[2,3-b]pyridin-3-yl]benzoic acid. MS m/e 345 [M+H]⁺.

### Example 197

4-[5-(3-aminophenyl)-1-H-pyrrolo-[2,3-b]pyridin-3-yl]-2-fluorobenzoic acid. MS m/e 348 [M+H]⁺.

### Example 198

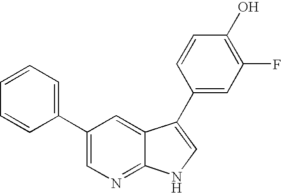

### Preparation of 2-fluoro-4-(5-phenyl-1H-pyrrolo[2,3-b]pyridin-3-yl)phenol

A solution of 3-[3-fluoro-4-(methyloxy)phenyl]-5-phenyl-1H-pyrrolo[2,3-b]pyridine (prepared as in Example 1 a-e) (77 mg, 0.24 mmole) in 5 ml of CH₂Cl₂ at 0° C. was treated with 1M BBr₃ in CH₂Cl₂ (0.7 ml, 0.7 mmole). The reaction was allowed to warm to 23° C. and stirred for 2 hours. MeOH was added, and the solvents were thoroughly evaporated. The oily residue was partitioned between H₂O and EtOAc, and the aqueous layer was neutralized to pH=6 with NaHCO₃. The organic layer was separated, dried, and the solvent evaporated. The residue was passed through a short Florisil column and eluted with 2% MeOH in EtOAc. The solvents were evaporated, and the residue was chromatographed (silica gel, 2% MeOH in CH₂Cl₂). The eluted product was crystallized from Et₂O to give the title compound (14 mg, 19%). ¹H NMR (400 MHz, CDCl₃) δ 10.60 (br s, 1H), 8.55 (s, 1H), 8.54 (s, 1H), 7.62 (d, J=1.2 Hz, 2H), 7.50 (m, 3H), 7.39 (m, 1H), 7.29 (m, 1H), 7.24 (d, J=1.2 Hz, 1H), 7.07 (t, J=8.4 Hz, 1H). MS m/e 305 [M+H]⁺.

### Example 199

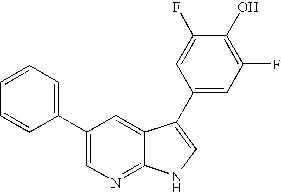

### Preparation of 2,6-difluoro-4-(5-phenyl-1H-pyrrolo[2,3-b]pyridin-3-yl)phenol

### a) 3-[3,5-difluoro-4-(methyloxy)phenyl]-1-[(4-methylphenyl)sulfonyl]-5-phenyl-1H-pyrrolo[2,3-b]pyridine

A mixture of 3-bromo-1-[(4-methylphenyl)sulfonyl]-5-phenyl-1H-pyrrolo[2,3-b]pyridine (188 mg, 0.44 mmol), 2-[3,5-difluoro-4-(methyloxy)phenyl]-4,4,5,5-tetramethyl-1,3,2-dioxaborolane (158 mg, 0.58 mmol), [1,1′-bis(diphenylphosphino)ferrocene]dichloropalladium(II) (40 mg), and K₂CO₃ (240 mg, 1.74 mmol) in dioxane (6 mL) and H₂O (2 mL) was heated at 80° C. for 1 hour. The mixture was diluted with H₂O and extracted with Et₂O. The extracts were washed with H₂O, dried and the solvent evaporated. The residue was purified by silica gel column chromatography (25% EtOAc/hexane) and gave the titled compound, 125 mg (58%). ¹H NMR (400 MHz, CDCl₃) δ 8.73 (s, 1H), 8.17 (d, 3H), 7.90 (s, 1H), 7.17-7.60 (m, 9H), 4.08 (s, 3H), 2.41 (s, 1H).

### b) 3-[3,5-difluoro-4-(methyloxy)phenyl]-5-phenyl-1H-pyrrolo[2,3-b]pyridine

A solution of 3-[3,5-difluoro-4-(methyloxy)phenyl]-1-[(4-methylphenyl)sulfonyl]-5-phenyl-1H-pyrrolo[2,3-b]pyridine (125 mg, 0.26 mmol) in THF (3 mL) was treated with tetrabutylammonium fluoride (1.0 mL of a 1N solution in THF) at 60° C. for 45 minutes. The reaction was diluted with H₂O, and extracted with Et₂O. The extracts were washed with H₂O, 0.001N NaOH, and H₂O, and were dried and the solvent evaporated to give the titled compound as a crystalline solid, 86 mg (98%). ¹H NMR (400 MHz, CDCl₃) δ 8.64 (s, 1H), 8.46 (s, 1H), 7.20-7.67 (m, 8H), 4.07 (s, 3H).

### c) 2,6-difluoro-4-(5-phenyl-1H-pyrrolo[2,3-b]pyridin-3-yl)phenol

The titled compound was prepared from 3-[3,5-difluoro-4-(methyloxy)phenyl]-5-phenyl-1H-pyrrolo[2,3-b]pyridine in a similar manner to that of Example 110. MS m/e 323 (M+H)⁺.

### Example 200

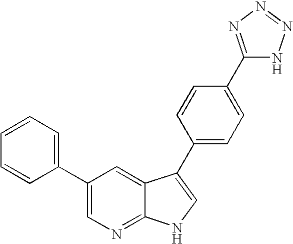

### Preparation of 5-phenyl-3-[4-(1H-tetrazol-5-yl)phenyl]-1H-pyrrolo[2,3-b]pyridine

### a) 1-[(4-methylphenyl)sulfonyl]-5-phenyl-3-[4-(1H-tetrazol-5-yl)phenyl]-1H-pyrrolo[2,3-b]pyridine

A mixture of 4-{1-[(4-methylphenyl)sulfonyl]-5-phenyl-1H-pyrrolo[2,3-b]pyridin-3-yl}benzonitrile (prepared as described in Example 1 a-d) (92 mg, 0.2 mmole), NaN₃ (65 mg, 1 mmole) and ZnBr₂ (56 mg, 0.25 mmole) in 5 ml of EtOH, 1 ml of THF and 1 ml of H₂O was placed in a sealed tube and heated at 140° C. for 30 hours. The reaction was cooled, concentrated, diluted with H₂O, acidified to pH=1 with HCl, and extracted with CH₂Cl₂. The extracts were washed with H₂O, dried, and the solvent evaporated. The residue was chromatographed over a Florisil column. An impurity was removed by eluting with 2% MeOH in EtOAc, and the product was eluted with a mixture of HOAc/MeOH/EtOAc (0.5/4/95) and provided the titled compound, 78 mg (79%). ¹H NMR (400 MHz, CDCl₃) δ 8.69 (s, 1H), 8.20 (m, 5H), 8.01 (s, 1H), 7.84 (d, J=8 Hz, 2H), 7.77 (d, J=8 Hz, 2H), 7.57 (t, J=7.2 Hz, 2H), 7.43 (d, J=7.2 Hz, 1H), 7.34 (d, J=8.4 Hz, 2H). 2.41 (s, 3H). MS m/e 493 [M+H]⁺.

### b) 5-phenyl-3-[4-(1H-tetrazol-5-yl)phenyl]-1H-pyrrolo[2,3-b]pyridine

A solution of 1-[(4-methylphenyl)sulfonyl]-5-phenyl-3-[4-(1H-tetrazol-5-yl)phenyl]-1H pyrrolo[2,3-b]pyridine (78 mg, 0.16 mmole) in 3 ml of THF was treated with 1M tetrabutylammonium fluoride in THF (1 ml, 1 mmole) and heated to 60° C. for 75 minutes. The reaction was cooled, diluted with H₂O and extracted with a mixture of Et₂O and EtOAc. The extracts were washed with H₂O, dried, treated with decolorizing carbon, filtered, and the filtrate evaporated. Trituration of the residue with Et₂O gave the titled compound, 27 mg (50%). ¹H NMR (400 MHz, DMSO-d₆) δ 8.61 (d, J=2.4 Hz, 1H), 8.56 (d, J=2.0 Hz, 1H), 8.13 (d, J=2.0 Hz, 2H), 8.11 (s, 1H), 8.07 (d, J=8.8 Hz, 2H), 7.81 (d, J=7.2 Hz, 2H), 7.52 (t, J=8.4 Hz, 2H), 7.40 (t, J=7.2 Hz, 1H). MS m/e 339 [M+H]⁺.

The compound of Examples 201 was prepared following the same general procedure described above for Example 200.

### Example 201

5-(2-naphthalenyl)-3-[4-(1H-tetrazol-5-yl)phenyl]-1H-pyrrolo[2,3-b]pyridine. MS m/e 389 (M+H)⁺.

### Example 202

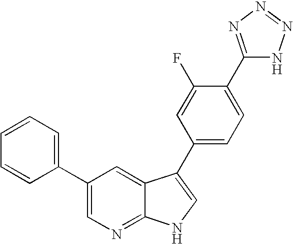

### Preparation of 3-[3-fluoro-4-(1H-tetrazol-5-yl)phenyl]-5-phenyl-1H-pyrrolo[2,3-b]pyridine

### a) {1-[(4-methylphenyl)sulfonyl]-5-phenyl-1H-pyrrolo[2,3-b]pyridin-3-yl}boronic acid

To a solution of 1-[(4-methylphenyl)sulfonyl]-5-phenyl-1H-pyrrolo[2,3-b]pyridine (416 mg, 1.2 mmol) in HOAc (15 mL) and 1 drop of HClO₄ was added Hg(OAc)₂ (380 mg, 1.2 mmol) and the reaction was stirred for 60 hours. The reaction was diluted with H₂O (50 mL), the solid was filtered, washed with H₂O and dried under vacuum. A solution of this solid in THF (15 ml) was added to a solution of BH₃ (10 mL of a 1N solution in THF), and the reaction, which now contained suspended solids, was stirred for 1 hour. H₂O (5 mL) was cautiously added, and the reaction mixture was stirred an additional 1 hour and concentrated. The residue was taken up in EtOAc and filtered. The filtrate was washed with water, dried and the solvent removed to give the titled compound as an off white powder, 383 mg (81%). MS m/e 393 [M+H]⁺.

### b) 5-(4-bromo-2-fluorophenyl)-1H-tetrazole

A solution of 4-bromo-2-fluorobenzonitrile (600 mg, 3 mmol) in EtOH (20 mL) was treated with NaN₃ (650 mg, 10 mmol) and ZnBr₂ (810 mg, 3.6 mmol) and the mixture was heated in a sealed tube at 120° c. for 20 hours. The reaction mixture was diluted with H₂O and extracted with Et₂O. The extracts were washed with H₂O, dried and the solvent evaporated and gave the titled compound, 675 mg (93%). ¹H NMR (400 MHz, CDCl₃) δ 8.02 (m, 1H), 7.90 (m, 1H), 7.68 (d, 1H).

### c) 3-[3-fluoro-4-(1H-tetrazol-5-yl)phenyl]-1-[(4-methylphenyl)sulfonyl]-5-phenyl-1H-pyrrolo[2,3-b]pyridine

A solution of 5-(4-bromo-2-fluorophenyl)-1H-tetrazole (125 mg, 0.51 mmol), {1-[(4-methylphenyl)sulfonyl]-5-phenyl-1H-pyrrolo[2,3-b]pyridin-3-yl}boronic acid (225 mg, 0.57 mmol), K₂CO₃ (315 mg, 2.28 mmol) and bis(diphenylphosphino)ferrocene]dichloropalladium(II) (40 mg) in dioxane (20 mL) and H₂O (5 mL) was heated at 80° C. for 3 hours. The reaction mixture was diluted with H₂O and extracted with Et₂O. The extracts were washed with H₂O, dried and the solvent evaporated. Florisil chromatography with a mixture of 2% HOAc in EtOAc gave the titled compound, 145 mg (55%). MS m/e 511 [M+H]⁺.

### d) 3-[3-fluoro-4-(1H-tetrazol-5-yl)phenyl]-5-phenyl-1H-pyrrolo[2,3-b]pyridine

A solution of 3-[3-fluoro-4-(1H-tetrazol-5-yl)phenyl]-1-[(4-methylphenyl)sulfonyl]-5-phenyl-1H-pyrrolo[2,3-b]pyridine (140 mg, 0.27 mmol) in THF (3 mL) was treated with tetrabutylammonium fluoride (1.5 mL of a 1N solution in THF) at 60° C. for 90 minutes. The reaction mixture was concentrated, diluted with H₂O and extracted with EtOAc. The extracts were washed with H₂O at pH 3, dried and the solvent evaporated. Trituration of the residue with a mixture of MeCN and MeOH gave the titled compound, 37 mg (38%). MS m/e 357 [M+H]⁺.

### Example 203

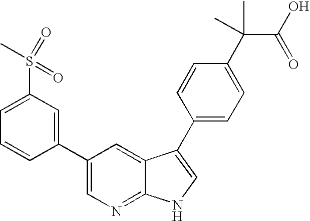

### Preparation of 2-methyl-2-(4-{5-[3-(methylsulfonyl)phenyl]-1H-pyrrolo[2,3-b]pyridin-3-yl}phenyl)propanoic acid

[1,1′-Bis(diphenylphosphino)ferrocene]dichloropalladium(II) complex with dichloromethane (30 mg, 0.04 mmol, 0.12 equiv) was added to a suspension of 3-bromo-5-[3-(methylsulfonyl)phenyl]-1H-pyrrolo[2,3-b]pyridine (0.152 g, 0.30 mmol, 1 equiv), methyl 2-methyl-2-[4-(4,4,5,5-tetramethyl-1,3,2-dioxaborolan-2-yl)phenyl]propanoate, (0.1 g, 0.33 mmol, 1.1 equiv) and potassium carbonate (0.13 g, 9.0 mmol, 3 equiv) in 2.5:1 dioxane/water (4 mL). The reaction flask was equipped with a water-cooled condenser, and the reaction mixture was heated to reflux under a nitrogen atmosphere. After three hours, a solution of 2N NaOH (1 mL) and MeOH (2 mL) was added and the reaction mixture was stirred at 80° C. for 12 h. The mixture was cooled to room temperature and acidified with concentrated HCl. The mixture was then filtered through a pad of Celite, and the filtrate was partitioned between ethyl acetate (10 mL) and water (10 mL). The layers were separated, and the aqueous layer was further extracted with ethyl acetate. The combined organics were dried over sodium sulfate and were concentrated. The crude product was purified via HPLC to give the product (16.8 mg, 11%) as a light yellow solid. ¹H-NMR (DMSO-d₆) δ: 1.527 (6H, s), 3.337 (3H, s), 7.442 (2H, d, J=8.4 Hz), 7.75 (3H, m), 7.923 (2H, m), 8.17 (1H, d, J=7.6 Hz), 8.281 (1H, s), 8.555 (1H, d, J=1.6 Hz), 8.671 (1H, d, J=1.6 Hz), 12.105 (1H, s), 12.367 (1H, s); MS m/e 407.4 [M+H]⁺.

### Examples 204-220

Compounds of Examples 204-220 were prepared following the same general procedure described above for Example 203.

### Example 204

2-{4-[5-(2-naphthalenyl)-1H-pyrrolo[2,3-b]pyridin-3-yl]phenyl}propanoic acid. MS m/e 393.2 [M+H]⁺.

### Example 205

2-(4-{5-[3-(aminocarbonyl)phenyl]-1H-pyrrolo[2,3-b]pyridin-3-yl}phenyl)-2-methylpropanoic acid. MS m/e 400.4 [M+H]⁺.

### Example 206

2-{4-[5-(3-cyanophenyl)-1H-pyrrolo[2,3-b]pyridin-3-yl]phenyl}-2-methylpropanoic acid. MS m/e 382.4 [M+H]⁺.

### Example 207

2-methyl-2-{4-[5-(6-quinolinyl)-1H-pyrrolo[2,3-b]pyridin-3-yl]phenyl}propanoic acid. MS m/e 408.4 [M+H]⁺.

### Example 208

2-methyl-2-{4-[5-(2-naphthalenyl)-1H-pyrrolo[2,3-b]pyridin-3-yl]phenyl}propanoic acid. MS m/e 435.2 [M+H]⁺.

### Example 209

2-methyl-2-[4-(5-{3-[(methylsulfonyl)amino]phenyl}-1H-pyrrolo[2,3-b]pyridin-3-yl)phenyl]propanoic acid. MS m/e 450.2 [M+H]⁺.

### Example 210

2-methyl-2-[4-(5-{4-[(methylsulfonyl)amino]phenyl}-1H-pyrrolo[2,3-b]pyridin-3-yl)phenyl]propanoic acid. MS m/e 450.2 [M+H]⁺.

### Example 211

2-methyl-2-(4-{5-[6-(methyloxy)-2-naphthalenyl]-1H-pyrrolo[2,3-b]pyridin-3-yl}phenyl)propanoic acid. MS m/e 437.4 [M+H]⁺.

### Example 212

2-methyl-2-{4-[5-(5-quinolinyl)-1H-pyrrolo[2,3-b]pyridin-3-yl]phenyl}propanoic acid. MS m/e 408.0 [M+H]⁺.

### Example 213

2-methyl-2-{4-[5-(3-quinolinyl)-1H-pyrrolo[2,3-b]pyridin-3-yl]phenyl}propanoic acid. MS m/e 408.2 [M+H]⁺.

### Example 214

2-{4-[5-(6-quinolinyl)-1H-pyrrolo[2,3-b]pyridin-3-yl]phenyl}propanoic acid. MS m/e 394.2 [M+H]⁺.

### Example 215

2-{4-[5-(5-quinolinyl)-1H-pyrrolo[2,3-b]pyridin-3-yl]phenyl}propanoic acid. MS m/e 394.0 [M+H]⁺.

### Example 216

2-{4-[5-(3-quinolinyl)-1H-pyrrolo[2,3-b]pyridin-3-yl]phenyl}propanoic acid. MS m/e 394.4 [M+H]⁺.

### Example 217

2-(4-{5-[3-(methylsulfonyl)phenyl]-1H-pyrrolo[2,3-b]pyridin-3-yl}phenyl)propanoic acid. MS m/e 421.0 [M+H]⁺.

### Example 218

2-(4-{5-[6-(methyloxy)-2-naphthalenyl]-1H-pyrrolo[2,3-b]pyridin-3-yl}phenyl)propanoic acid. MS m/e 423.0 [M+H]⁺.

### Example 219

2-methyl-2-[4-(5-phenyl-1H-pyrrolo[2,3-b]pyridin-3-yl)phenyl]propanoic acid. MS m/e 357.2 [M+H]⁺.

### Example 220

2-methyl-2-{4-[5-(2-naphthalenyl)-1H-pyrrolo[2,3-b]pyridin-3-yl]phenyl}propanoic acid. MS m/e 407.4 [M+H]⁺.

### Example 221

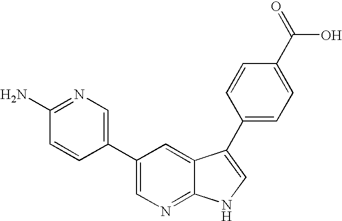

### Preparation of 4-[5-(6-amino-3-pyridinyl)-1H-pyrrolo[2,3-b]pyridin-3-yl]benzoic acid

### a) tert-butyl[5-(1H-pyrrolo[2,3-b]pyridin-5-yl)pyridin-2-yl]carbamate

A solution of tert-butyl (5-iodopyridin-2-yl)carbamate (195 mg, 0.61 mmol), 5-(4,4,5,5-tetramethyl-1,3,2-dioxaborolan-2-yl)-1H-pyrrolo[2,3-b]pyridine (165 mg, 0.68 mmol), bis(diphtenylphosphino)ferrocene]dichloropalladium(II) (40 mg) and K₂CO₃ (282 mg, 2.04 mmol) in dioxane (20 mL) and H₂O (4 mL) was heated at 75° C. for 1 hour. The reaction was diluted with H₂O and extracted with Et₂O. The extracts were washed with H₂O, dried and the solvent evaporated. The residue was purified by ISCO chromatography (12 g silica column, dichloromethane for 5 min, dichloromethane grading to 2% methanol/dichloromethane over 1 minute, 2% methanol/dichloromethane for 40 minutes) and afforded the titled compound, 70 mg (37%). MS m/e 311 [M+H]⁺.

### b) tert-butyl[5-(3-bromo-1H-pyrrolo[2,3-b]pyridin-5-yl)pyridin-2-yl]carbamate

A solution of tert-butyl [5-(1H-pyrrolo[2,3-b]pyridin-5-yl)pyridin-2-yl]carbamate (240 mg, 0.77 mmol) in a mixture of THF (10 mL) and DMF (5 mL) was treated with N-bromosuccinimide (158 mg, 0.89 mmol) for 2 hours at 22° C. The reaction mixture was concentrated, diluted with H₂O, and extracted with a mixture of Et₂O and EtOAc. The extracts were washed with H₂O and 0.2N NaOH, and were dried and concentrated. The residue crystallized from the solvent mixture during concentration to gave 230 mg of the title compound (77%). ¹H NMR (400 MHz, CDCl₃) δ 9.90 (s, 1H), 8.65 (m, 1H), 8.62 (d, 1H), 8.07 (d, 1H), 7.90 (d, 1H), 7.78 (d, 1H), 1.50 (s, 9H).

### c) tert-butyl 3-bromo-5-(6-[(tert-butoxycarbonyl)amino]pyridin-3-yl]-1H-pyrrolo[2,3-b]pyridine-1-carboxylate

A solution of tert-butyl [5-(3-bromo-1H-pyrrolo[2,3-b]pyridin-5-yl)pyridin-2-yl]carbamate (230 mg, 0.59 mmol) and 4-dimethylaminopyridine (7.5 mg, 0.06 mmol) in THF (20 mL) was treated with di-t-butyl dicarbonate (138 mg, 0.64 mmol) at 22° C. for 15 minutes. The reaction mixture was diluted with Et₂O and washed with 30 mL of 0.03N HCl. The Et₂O layer was dried, and the solvent evaporated. The residue was purified by ISCO chromatography (12 g silica column, 10% EtOac/hexane for 5 minutes grading to 20% EtOAc/hexane over 1 minute, then 20% EtOAc/hexane for 15 minutes) and afforded the titled compound as a white, crystalline solid, 197 mg (68%). ¹H NMR (400 MHz, CDCl₃) δ 9.09 (s, 1H), 8.73 (d, 1H), 8.63 (t, 1H), 8.18 (m, 1H), 7.98 (m, 2H), 7.78 (s, 1H), 1.70 (s, 9H), 1.57 (s, 9H).

### d) tert-butyl 5-[6-[(tert-butoxycarbonyl)amino]pyridin-3-yl]-3-[4-(tert-butoxycarbonyl)phenyl]-1H-pyrrolo[2,3-b]pyridine-1-carboxylate

A solution of tert-butyl 3-bromo-5-{6-[(tert-butoxycarbonyl)amino]pyridin-3-yl}-1H-pyrrolo[2,3-b]pyridine-1-carboxylate (197 mg, 0.4 mmol), 4-t-butoxycarbonylphenyl boronic acid (111 mg, 0.5 mmol), K₂CO₃ (165 mg, 1.2 mmol) and bis(diphenylphosphino)ferrocene]dichloropalladium(II) (50 mg) in dioxane (20 mL) and H₂O (4 mL) was heated at 80° C. for 90 minutes. The reaction mixture was diluted with H₂O and extracted with Et₂O. The extracts were washed with H₂O, dried and the solvent evaporated. The residue was purified by ISCO chromatography (12 g silica column, 10% EtOAc/hexane for 5 minutes grading to 20% EtOAc/hexane over 1 minute, then 20% EtOAc/hexane for 15 minutes) and afforded the titled compound as a white, crystalline solid, 124 mg (53%). ¹H NMR (400 MHz, CDCl₃) δ 8.88 (d, 1H), 8.58 (d, 1H), 8.40 (s, 1H), 8.28 (d, 1H), 8.16 (m, 3H), 7.92 (m, 1H), 7.70 (d, 2H), 1.74 (s, 9H), 1.64 (s, 9H), 1.55 (s, 9H).

### e) 4-[5-(6-aminopyridin-3-yl)-1H-pyrrolo[2,3-b]pyridin-3-yl]benzoic acid

A solution of tert-butyl 5-{6-[(tert-butoxycarbonyl)amino]pyridin-3-yl}-3-[4-(tert-butoxycarbonyl)phenyl]-1H-pyrrolo[2,3-b]pyridine-1-carboxylate (124 mg, 0.21 mmol) in CH₂Cl₂ (2 mL) was treated with TFA (2 mL) at 22° C. for 1 hour. All solvent was thoroughly evaporated, and the residue was triturated with Et₂O to give the titled compound as the trifluoroacetate salt, 84 mg (72%) MS m/e 331 [M+H]⁺.

### Example 222

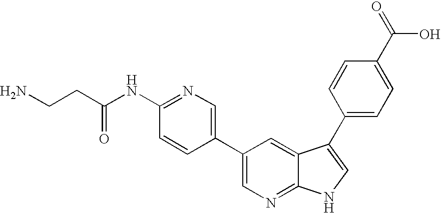

### Preparation of 4-(5-[6-(β-alanylamino)-3-pyridinyl]-1H-pyrrolo[2,3-b]pyridin-3-yl)benzoic acid

### a) 1,1-dimethylethyl{3-[(5-iodo-2-pyridinyl)amino]-3-oxopropyl}carbamate

A solution of 2-amino-5-iodopyridine (770 mg, 3.5 mmol), N-t-butoxycarbonyl-β-alanine (662 mg, 3.5 mmol) and 1-cyclohexyl-3-(2-morpholinoethyl)carbodiimide metho-p-toluene sulfonate (1.63 gm, 3.85 mmol) in CH₂Cl₂ (40 mL) was stirred for 1.8 hours. The reaction mixture was washed with aqueous Na₂CO₃ and 0.1N HCl. The organics were dried and the solvent evaporated. The residue was crystallized from acetonitrile and gave the titled compound, 240 mg (18%). ¹H NMR (400 MHz, CDCl₃). 810.58 (s, 1H), 8.50 (s, 1H), 8.12 (d, 1H), 7.96 (d, 1H), 6.85 (s, 1H), 3.20 (m, 2H), 2.50 (m, 2H), 1.36 (s, 9H).

### b) 1,1-dimethylethyl (3-oxo-3-{[5-(1H-pyrrolo[2,3-b]pyridin-5-yl)-2-pyridinyl]amino}propyl)carbamate

A solution of 1,1-dimethylethyl {3-[(5-iodo-2-pyridinyl)amino]-3-oxopropyl}carbamate (233 mg, 0.8 mmol), 5-(4,4,5,5-tetramethyl-1,3,2-dioxaborolan-2-yl)-1H-pyrrolo[2,3-b]pyridine (240 mg, 1 mmol), bis(diphenylphosphino)ferrocene]dichloropalladium(II) (50 mg), and K₂CO₃ 414 mg, 3 mmol) in dioxane (25 mL) and H₂O (95 mL) was heated at 80° C. for 1 hour. The reaction was diluted with H₂O and extracted with Et₂O. The extracts were washed with H₂O, dried and the solvent evaporated. The residue was purified by ISCO chromatography (12 g silica column, 2% MeOH/dichloromethane for 40 min, grading to 5% methanol/dichloromethane over 10 minutes) and afforded the titled compound as a white crystalline solid, 222 mg (95%). ¹H NMR (400 MHz, DMSO-d₆) δ 11.75 (s, 1H), 10.55 (s, 1H), 8.67 (s, 1H), 8.54 (d, 1H), 8.26 (s, 1H), 8.15 (m, 2H), 7.53 (s, 1H), 6.88 (s, 1H), 6.50 (s, 1H), 3.24 (m, 2H), 2.57 (m, 2H), 1.38 (s, 9H)

### c) 1,1-dimethylethyl (3-{[5-(3-bromo-1H-pyrrolo[2,3-b]pyridin-5-yl)-2-pyridinyl]amino}-3-oxopropyl)carbamate

A solution of 1,1-dimethylethyl (3-oxo-3-{[5-(1H-pyrrolo[2,3-b]pyridin-5-yl)-2-pyridinyl]amino}propyl)carbamate (220 mg, 0.78 mmol) in THF (8 mL) and DMF (4 mL) was treated with N-bromosuccinimide (153 mg, 0.86 mmol) for 30 minutes. The solvent was concentrated, and the residue taken up in Et₂O, washed with H₂O and 0.01N NaOH. The solvent was dried and evaporated, and gave the titled compound, 157 mg, (56%). MS m/e 361 [M+H]⁺.

### d) 1,1-dimethylethyl 3-bromo-5-{6-[(N{[(1,1-dimethylethyl)oxy]carbonyl}-β-alanyl)amino]-3-pyridinyl}-1H-pyrrolo[2,3-b]pyridine-1-carboxylate

A solution of 1,1-dimethylethyl (3-{[5-(3-bromo-1H-pyrrolo[2,3-b]pyridin-5-yl)-2-pyridinyl]amino}-3-oxopropyl)carbamate (157 mg, 0.44 mmol) and 4-dimethylaminopyridine (6 mg, 0.05 mmol) in THF (20 mL) was treated with di-t-butyl dicarbonate (105 mg, 0.48 mmol) at 22° C. for 15 minutes. The reaction was diluted with Et₂O and washed with 30 mL of 0.03N HCl. The Et₂O was dried, and the solvent evaporated. The residue was purified by ISCO chromatography (12 g silica column, 20% EtOAc/hexane for 10 minutes grading to 50% EtOAc/hexane over 10 minutes, then 50% EtOAc/hexane for 20 minutes) and afforded the titled compound as a white, crystalline solid, 97 mg (50%). ¹H NMR (400 MHz, CDCl₃) δ 8.72 (s, 1H), 8.56 (s, 1H), 8.30 (m, 1H), 7.97 (m, 2H), 7.73 (s, 1H), 5.30 (s, broad, 1H), 3.54 (m, 2H), 2.72 (m, 2H), 1.69 (s, 9H), 1.44 (s, 9H).

### e) 1,1-dimethylethyl 5-{6-[(N-{[(1,1-dimethylethyl)oxy]carbonyl}-β-alanyl)amino]-3-pyridinyl}-3-(4-{[(1,1-dimethylethyl)oxy]carbonyl}phenyl)-1H-pyrrolo[2,3-b]pyridine-1-carboxylate

A solution of 1,1-dimethylethyl 3-bromo-5-{6-[(N-([(1,1-dimethylethyl)oxy]carbonyl}-β-alanyl)amino]-3-pyridinyl}-1H-pyrrolo[2,3-b]pyridine-1-carboxylate (95 mg, 0.2 mmol), 4-t-butoxycarbonylphenyl boronic acid (56 mg, 0.25 mmol), K₂CO₃ (97 mg, 0.7 mmol) and bis(diphenylphosphino)ferrocene]dichloropalladium(II) (50 mg) in dioxane (20 mL) and H₂O (4 mL) was heated at 80° C. for 90 minutes. The reaction was diluted with H₂O and extracted with Et₂O. The extracts were washed with H₂O, dried and the solvent evaporated. The residue was purified by ISCO chromatography (12 g silica column, 25% EtOAc/hexane for 10 minutes, then grading to 50% EtOAc/hexane over 1 minute, then 50% EtOAc/hexane for 25 minutes) and afforded the titled compound 96 mg (78%). ¹H NMR (400 MHz, CDCl₃) δ 8.76 (s, 1H), 8.56 (s, 1H), 8.34 (m, 2H), 8.28 (s, 1H), 8.16 (m, 2H), 7.99 (m, 1H), 7.90 (s, 1H), 7.70 (d, 1H), 5.24 (s, 1H), 3.54 (m, 2H), 2.71 (m, 2H), 1.74 (s, 9H0, 1.65 (s, 9H), 1.45 (s, 9H).

### f). 4-{5-[6-(β-alanylamino)-3-pyridinyl]-1H-pyrrolo[2,3-b]pyridin-3-yl}benzoic acid

A solution of 1,1-dimethylethyl 5-(6-[(N-([(1,1-dimethylethyl)oxy]carbonyl)-β-alanyl)amino]-3-pyridinyl)-3-(4-{[(1,1-dimethylethyl)oxy]carbonyl}phenyl)-1H-pyrrolo[2,3-b]pyridine-1-carboxylate (94 mg, 0.17 mmol) in CH₂Cl₂ (1 mL) was treated with TFA (1 mL) for 1 hour. The solvents were thoroughly evaporated, and the residue was triturated with Et₂O and gave the titled compound as the trifluoroacetate salt, 38 mg (56%). MS m/e 402 [M+H]⁺.

### Example 223

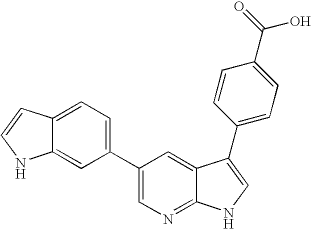

### Preparation of 4-(5-(6-indolyl)-1H-pyrrolo[2,3-b]pyridin-3-yl)benzoic acid

### a) Preparation of 1-(1,1-dimethylethyloxycarbonyl)-6-(5-[1H-pyrrolo[2,3-b]pyridin-3-yl])indole

A mixture of 5-bromo-7-azaindole (100 mg, 0.51 mmol), 1-(1,1-dimethylethyloxycarbonyl)-6-(4,4,5,5-tetramethyl-1,3,2-dioxaborolan-2-yl)indole (192 mg, 0.56 mmol), potassium carbonate (210 mg, 1.5 mmol), and [1,1′-bis(diphenylphosphino)ferrocene]dichloropalladium(II) complex with dichloromethane (21 mg, 0.025 mmol) in 2.5:1 dioxane/water (5.1 mL) was heated to 80° C. in a microwave oven for 5 m. The reaction mixture was diluted with ethyl acetate (25 mL) and washed once with saturated aqueous sodium chloride (25 mL). The organics were dried over anhydrous sodium sulfate and were concentrated. Purification of the residue by flash column chromatography (20% ethyl acetate in hexanes grading to 50% ethyl acetate in hexanes) provided 1-(1,1-dimethylethyloxycarbonyl)-6-(5-[1H-pyrrolo[2,3-b]pyridin-3-yl])indole (70 mg, 40%) as a white foam. MS m/e 334.2 [M+H]⁺.

### b) Preparation of 1-(1,1-dimethylethyloxycarbonyl)-6-(3-bromo-5-[1H-pyrrolo[2,3-b]pyridin-3-yl])indole

N-bromosuccinimide (37 mg, 0.21 mmol) was added as a suspension in dichloromethane (1 mL) to a solution of 1-(1,1-dimethylethyloxycarbonyl)-6-(5-[1H-pyrrolo[2,3-b]pyridin-3-yl])indole (65 mg, 0.2 mmol) and N,N-diisolpropylethylamine (35 uL, 26 mg, 0.2 mmol) in dichloromethane (2 mL). The resultant yellow solution was stirred at room temperature for 24 h, and then three more portions of N-bromosuccinimide (37 mg, 0.21 mmol) and N,N-diisolpropylethylamine (35 uL, 26 mg, 0.2 mmol) were added at 1-hour intervals. The reaction mixture was diluted with dichloromethane (10 mL) and washed with 1N NaOH (10 mL). The aqueous layer was extracted with dichloromethane (2 5-mL portions), and the combined organics were dried over anhydrous sodium sulfate and were concentrated. Purification of the residue by flash column chromatography (10% ethyl acetate in dichloromethane grading to 50% ethyl acetate in dichloromethane) provided 1-(1,1-dimethylethyloxycarbonyl)-6-(3-bromo-5-[1H-pyrrolo[2,3-b]pyridin-3-yl])indole (60 mg, 73%) as an orange oil. MS m/e 271.4 [M-t-BuO]⁺.

### c) Preparation of 1-(1,1-dimethylethyloxycarbonyl)-6-(1-[1,1-dimethylethyloxycarbonyl]-3-bromo-5-[1H-pyrrolo[2,3-b]pyridin-3-yl])indole

Prepared as described in Example 171(a).

### d) Preparation of 4-(5-(1-[1,1-dimethylethyloxycarbonyl]-6-indolyl)-(1-[1,1-dimethylethyloxycarbonyl]-1H-pyrrolo[2,3-b]pyridin-3-yl)benzoic acid tert-butyl ester

Prepared as described in Example 171(b).

### e) Preparation of 4-(5-(6-indolyl)-1H-pyrrolo[2,3-b]pyridin-3-yl)benzoic acid

Trifluoroacetic acid (0.2 mL) was added to a solution of 4-(5-(1-[1,1-dimethylethyloxycarbonyl]-6-indolyl)-(1-[1,1-dimethylethyloxycarbonyl]-1H-pyrrolo[2,3-b]pyridin-3-yl)benzoic acid tert-butyl ester (23 mg, 0.038 mmol) in dichloromethane (2 mL). The reaction mixture was stirred at room temperature for 18 h, and then concentrated in vacuo. The residue was stirred with dichloromethane (2 mL), and the insolubles were collected by filtration to provide 4-(5-(6-indolyl)-1H-pyrrolo[2,3-b]pyridin-3-yl)benzoic acid as a red powder (7.2 mg, 54%). ¹H NMR (400 MHz, DMSO-d₆) δ 11.1 (s, 1H), 8.6 (s, 1H), 8.5 (s, 1H), 8.1 (s, 1H), 8.0 (m, 4H), 7.7 (s, 1H), 7.6 (d, J=8.0 Hz, 1H), 7.4 (m, 2H), 6.5 (s, 1H); MS m/e 354.2 [M+H]⁺.

### Example 224

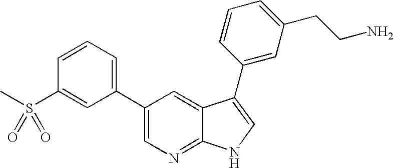

### Preparation of [2-(3-{5-[3-(methylsulfonyl)phenyl]-1H-pyrrolo[2,3-b]pyridin-3-yl}phenyl)ethyl]amine

A solution of 1,1-dimethylethyl [2-(3-{5-[3-(methylsulfonyl)phenyl]-1H-pyrrolo[2,3-b]pyridin-3-yl}phenyl)ethyl]carbamate (prepared as described in Example MHX, 0.0663 g, 0.135 mmol) in CH₂Cl₂ (2 mL) was treated with trifluoroacetic acid (0.5 mL) and stirred for two hours. The solvent was removed under rotary evaporation, and excess trifluoroacetic acid was removed using a Genevac vacuum system to give the title compound (0.0821 g, 98%) as the bis-TFA salt in the form of an off-white solid. ¹H NMR (400 MHz, DMSO-d₆) δ 8.68 (d, J=2 Hz, 1H), 8.55 (d, J=2 Hz, 1H), 8.26 (d, J=1.6 Hz, 1H), 8.16 (dd, J=1.2, 8.0 Hz, 1H), 7.94 (m, 1H), 7.73 (m, 4H), 7.45 (m, 1H), 7.20 (m, 1H), 3.33 (s, 3H), 3.15 (m, 2H), 2.96 (m, 2H). LCMS m/e 392.2 [M+H]⁺.

### Examples 225-228

Compounds in Examples 225-228 were prepared following the same general procedure described above for Example 224.

### Example 225

N-(3-{3-[3-(2-aminoethyl)phenyl]-1H-pyrrolo[2,3-b]pyridin-5-yl}phenyl)methanesulfonamide. MS m/e 407.6 [M+H]⁺.

### Example 226

N-(4-{3-[3-(2-aminoethyl)phenyl]-1H-pyrrolo[2,3-b]pyridin-5-yl}phenyl)methanesulfonamide. MS m/e 407.4 [M+H]⁺.

### Example 227

4-{5-[3-(2-aminoethyl)phenyl]-1-H-pyrrolo-[2,3-b]pyridin-3-yl}benzoic acid. MS m/e 358 [M+H]⁺.

### Example 228

4-{5-[3-(2-aminoethyl)phenyl]-1-H-pyrrolo-[2,3-b]pyridin-3-yl}-2-methylbenzoic acid. MS m/e 372 [M+H]⁺.

### Example 229

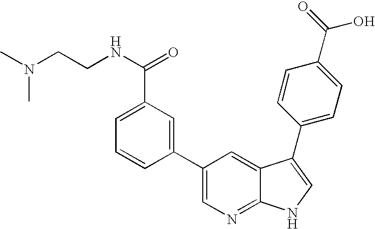

### Preparation of 4-{5-[3-({[2-(dimethylamino)ethyl]amino}carbonyl)phenyl]-1H-pyrrolo[2,3-b]pyridin-3-yl}benzoic acid

To a solution of 3-[3-(4-{[(1,1-dimethylethyl)oxy]carbonyl}phenyl)-1H-pyrrolo[2,3-b]pyridin-5-yl]benzoic acid (0.050 g, 0.12 mmol) in CH₂Cl₂ (1 mL) was added N,N-dimethylethylenediamine (0.015 mL, 0.13 mmol) and 1-ethyl-3-(3-dimethylaminopropyl)carbodiimide hydrochloride (0.028 g, 0.14 mmol). The reaction mixture was stirred for 18 hours and then was diluted with water (2 mL). The aqueous layer was extracted twice with CH₂Cl₂ (2 mL). The combined organic extracts were dried over Na₂SO₄, filtered, and concentrated. The crude product was purified by flash silica gel chromatography (10% MeOH/CH₂Cl₂). The product fractions were concentrated to an oil, which was dissolved in 1 mL CH₂Cl₂ and 1 mL trifluoroacetic acid. The reaction mixture was stirred for 18 hours and then concentrated. The mixture was concentrated several times from CH₂Cl₂ and then put under high vacuum to obtain the title compound as an orange foam (0.036 g, 55%). ¹H NMR (400 MHz, MeOD) δ 8.71 (s, 1H), 8.64 (s, 1H), 8.22 (s, 1H), 8.13 (d, J=7.6 Hz, 2H), 7.94 (t, J=6.4 Hz, 3H), 7.86 (d, J=8.0 Hz, 2H), 7.64 (t, J=7.6 Hz, 1H), 3.83 (t, J=5.2 Hz, 2H), 3.44 (t, J=5.2 Hz, 2H), 3.02 (s, 6H). LCMS m/e 429.2 [M+H]⁺.

### Example 230

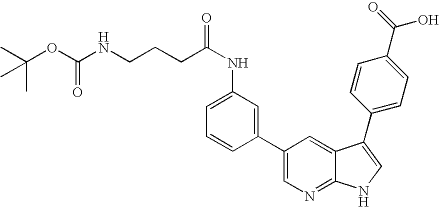

### Preparation of 4-(5-(3-[4-(1,1-dimethylethyloxycarbonyl)aminobutanoyl]amino)phenyl-1H-pyrrolo[2,3-b]pyridin-3-yl)benzoic acid

### a) 5-(3-[4-(1,1-dimethylethyloxycarbonyl)aminobutanoyl]amino)phenyl-1H-pyrrolo[2,3-b]pyridine

Prepared as described in Example 229 starting with 5-(3-aminophenyl)-1H-pyrrolo[2,3-b]pyridine and 4-tbutoxycarbonylaminobutyric acid. MS m/e 295.4 [M-t-BuOCO]⁺.

### b) 3-bromo-5-(3-[4-(1,1-dimethylethyloxycarbonyl)aminobutanoyl]amino)phenyl-1H-pyrrolo[2,3-b]pyridine

Prepared as described in Example 223(b).

### c) 1-(1,1-dimethylethyloxycarbonyl)-3-bromo-5-(3-[4-(2,2-dimethylethyloxycarbonyl)aminobutanoyl]amino)phenyl-1H-pyrrolo[2,3-b]pyridine

Prepared as described in 168(a) to provide 1-(1,1-dimethylethyloxycarbonyl)-3-bromo-5-(3-[4-(2,2-dimethylethyloxycarbonyl)aminobutanoyl]amino)phenyl-1H-pyrrolo[2,3-b]pyridine. MS m/e 573.2 [M]⁺.

### d) 4-(5-(3-[4-(1,1-dimethylethyloxycarbonyl)aminobutanoyl]amino)phenyl-1H-pyrrolo[2,3-b]pyridin-3-yl)benzoic acid

Prepared as described in Example 168(b). ¹H NMR (400 MHz, CD₃OD) 8.5 (s, 2H), 8.1 (d, J=7.6 Hz, 2H), 7.9 (s, 1H), 7.8 (m, 3H), 7.6 (br s, 1H), 7.5 (m, 2H), 3.2 (t, J=5.6 Hz, 2H), 2.5 (t, J=6.8 Hz, 2H), 1.9 (t, m, 2H), 1.4 (s, 9H); MS m/e 415.4 [M-t-BuOCO]⁺.

### Examples 231-232

Compounds in Examples 231 and 232 were prepared following the same general procedure described above for Example 230.

### Example 231

4-(5-(3-[3-(1,1-dimethylethyloxycarbonyl)aminopropanoyl]amino)phenyl-1H-pyrrolo[2,3-b]pyridin-3-yl)benzoic acid. MS m/e 401.2 [M-t-BuOCO]⁺.

### Example 232

2-(2-propyl)-4-[5-(3-[3-(1,1-dimethylethyloxycarbonyl)aminopropanoyl]amino)phenyl-1H-pyrrolo[2,3-b]pyridin-3-yl]benzoic acid. MS m/e 543.4 [M+H]⁺.

### Example 233

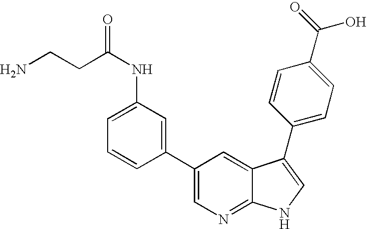

### Preparation of 4-{5-[3-(β-alanylamino)phenyl]-1H-pyrrolo[2,3-b]pyridin-3-yl}benzoic acid

Trifluoroacetic acid (0.5 mL) was added to a solution of 4-(5-(3-[3-(1,1-dimethylethyloxycarbonyl)aminopropanoyl]amino)phenyl-1H-pyrrolo[2,3-b]pyridin-3-yl)benzoic acid (32 mg, 0.064 mmol) in dichloromethane (1 mL). The resultant solution was stirred at room temperature for 7 h and the reaction mixture was concentrated in vacuo. The residue was concentrated in vacuo from toluene (5 mL), and then taken up in methanol. DOWEX-50WX2-400 ion exchange resin (50 mg, previously washed and dried) was added, and the mixture was stirred gently for ½ h. The resin was isolated by filtration and washed with dichloromethane and methanol. The product was released from the resin by washing with 4N ammonia in methanol (4 50-mL portions). The filtrate was concentrated in vacuo to afford 4-{5-[3-(O-alanylamino)phenyl]-1H-pyrrolo[2,3-b]pyridin-3-yl}benzoic acid (3.2 mg, 12%). ¹H NMR (400 MHz, DMSO-d₆) δ 8.5 (s, 1H), 8.4 (s, 1H), 8.0 (m, 3H), 7.9 (s, 1H), 7.8 (d, J=8.4 Hz, 2H), 7.7 (d, J=6.4 Hz, 1H), 7.4 (m, 2H), 3.0 (m, 2H), 2.5 (m, 2H).

### Examples 234-238

Compounds in Examples 234-238 were prepared following the same general procedure described above for Example 233.

### Example 234

4-(5-{3-[(4-aminobutanoyl)amino]phenyl}-1H-pyrrolo[2,3-b]pyridin-3-yl)benzoic acid. MS m/e 415.0 [M+H]⁺.

### Example 235

4-{5-[3-(beta-alanylamino)phenyl]-1H-pyrrolo[2,3-b]pyridin-3-yl}-2-methylbenzoic acid. MS m/e 415.0 [M+H]⁺.

### Example 236

4-{5-[3-(beta-alanylamino)phenyl]-1H-pyrrolo[2,3-b]pyridin-3-yl}-2-(1-methylethyl)benzoic acid. MS m/e 443.2 [M+H]⁺.

### Example 237

4-[5-(3-{[(2-aminoethyl)amino]carbonyl}phenyl)-1H-pyrrolo[2,3-b]pyridin-3-yl]benzoic acid. MS m/e 401.4 [M+H]⁺.

### Example 238

4-[5-(3-{[(3-aminopropyl)amino]carbonyl}phenyl)-1H-pyrrolo[2,3-b]pyridin-3-yl]benzoic acid. MS m/e 415.4 [M+H]⁺.

### Example 239

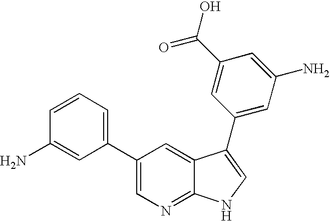

### Preparation of 3-amino-5-[5-(3-aminophenyl)-1-H-pyrrolo-[2,3-b]pyridin-3-yl]benzoic acid

### a) Preparation of 2,2,2-trifluoro-N-[3-(1-H-pyrrolo-[2,3-b]pyridin-5-yl)phenyl]acetamide

A solution of 3-(1-H-pyrrolo-[2,3-b]pyridin-5-yl)aniline ((1.1 g, 5.07 mmol) and diisopropylethylamine (1.76 mL, 10.1 mmol) in methylene chloride (20 mL) was cooled to 0° C. A solution of trifluoroacetic anhydride (1.12 g, 5.32 mmol) in chloroform (10 mL) was added dropwise to the reaction mixture. The reaction mixture was diluted with water (50 mL) and extracted with chloroform (2×50 mL). The combined organics were dried over anhydrous sodium sulfate and were concentrated to provide 2,2,2-trifluoro-N-[3-(1-H-pyrrolo-[2,3-b]pyridin-5-yl)phenyl]acetamide, which was used in the next reaction without further purification. ¹H NMR (400 MHz, CDCl₃) δ 8.46 (s, 1H), 8.25 (s, 1H), 7.96 (s, 1H), 7.70 (m, 1H), 7.56-7.45 (m, 3H), 6.59 (s, 1H). TLC (25% ethyl acetate in hexanes) Rf=0.3.

### b) Preparation of N-[3-(3-bromo-1H-pyrrolo[2,3-b]pyridin-5-yl)phenyl]-2,2,2-trifluoroacetamide

Prepared according to the general procedure given in Example 1(b).

### c) Preparation of N(3-{3-bromo-1-[4-methylphenyl)sulfonyl]-1-H-pyrrolo-[2,3-b]pyridin-5-yl}phenyl)-2,2,2-trifluoroacetamide

Prepared according to the general procedure described in Example 1(c).

### d) 3-amino-5-[5-(3-aminophenyl)-1-H-pyrrolo-[2,3-b]pyridin-3-yl]benzoic acid

Prepared according to the general procedure described in Example 1(d) followed by deprotection of both the trifluoroacetamide and the tosyl groups using the general procedure described in Example 1(e). ¹H NMR (400 MHz, CDCl₃) δ 8.55 (m, 2H), 7.88 (s, 1H), 7.80 (s, 1H), 7.69 (m, 1H), 7.61 (m, 2H), 7.57 (m, 2H), 7.28 (m, 1H).

### Examples 240-241

Compounds in Examples 240-241 were prepared following the same general procedure described above for Example 239.

### Example 240

3-amino-5-{5-[3-(aminomethyl)phenyl]-1-H-pyrrolo-[2,3-b]pyridin-3-yl}benzoic acid. MS m/e 358 [M+H]⁺.

### Example 241

4-{5-[3-(aminomethyl)phenyl]-1-H-pyrrolo-[2,3-b]pyridin-3-yl}-2-fluorobenzoic acid. MS m/e 362 [M+H]⁺.

### Example 242

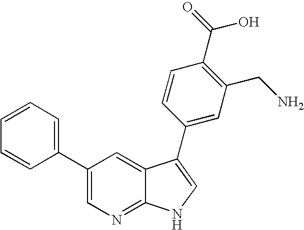

### Preparation of 2-(aminomethyl)-4-(5-phenyl-1H-pyrrolo[2,3-b]pyridin-3-yl)benzoic acid

### a) Preparation of 2-(azidomethyl)-4-(5-phenyl-1H-pyrrolo[2,3-b]pyridin-3-yl)benzoic acid

Methyl 2-(azidomethyl)-4-(5-phenyl-1H-pyrrolo[2,3-b]pyridin-3-yl)benzoate (0.24 g, 0.63 mmol) was dissolved in methanol/THF (1 mL) and hydrolyzed with 1N NaOH (1 mL) for 8 h. 1N HCl was added to bring the pH down to 3. A green precipitate was formed which was isolated and dried to give the pure product (0.2 g, 86%).

### b) 2-(aminomethyl)-4-(5-phenyl-1H-pyrrolo[2,3-b]pyridin-3-yl)benzoic acid

Triphenylphosphine (0.05 g, 0.2 mmol) was added to a solution of 2-(azidomethyl)-4-(5-phenyl-1H-pyrrolo[2,3-b]pyridin-3-yl)benzoic acid (0.05 g, 0.13 mmol) in THF (0.8 mL). Water (0.012 mL, 0.67 mmol) was added, followed by DMF (2 drops) for solubility. The reaction was stirred for 4 days, and then concentrated and purified by HPLC(XTerra Prep RP, 19 mL/min, 18 min, 10-99% CH₃CN/H₂O, 0.05% TFA) to obtain 0.017 g (38%) of 2-(aminomethyl)-4-(5-phenyl-1H-pyrrolo[2,3-b]pyridin-3-yl)benzoic acid. ¹H-NMR (MeOD) δ: 8.62 (d, 1H), 8.31 (d, 1H), 8.00 (m, 3H), 7.74 (d, 2H), 7.51 (m, 2H), 7.41 (m, 2H), 4.50 (s, 2H). MS m/e 344 [M+H]⁺.

### Example 243

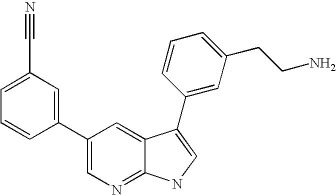

### Preparation of 3-{3-[3-(2-aminoethyl)phenyl]-1 pyrrolo[2,3-b]pyridin-5-yl}benzonitrile

1,1-Dimethylethyl (2-{3-[5-(3-cyanophenyl)-1H-pyrrolo[2,3-b]pyridin-3-yl]phenyl}ethyl)carbamate (prepared as described in Example 1 (a-e)) (0.18 g, 0.38 mmol) was treated with 4M HCl/dioxane (1 mL) at room temperature for 30 min. Solvent was removed and the residue was neutralized with 1N NaOH and partitioned between ethyl acetate and water. The organic layer was dried over sodium sulfate, concentrated and purified via HPLC to give the product (30 mg, 23%) as a yellow solid. ¹H-NMR (DMSO-d₆) δ:2.956 (2H, m), 3.145 (2H, m), 7.184 (1H, d, J=8.0 Hz), 7.447 (1H, t, J=7.6 Hz), 7.709 (3H, m), 7.855 (3H, m), 7.960 (1H, d, J=2.4 Hz), 8.147 (1H, m), 8.318 (1H, m) 8.563 (1H, d, J=2.0 Hz), 8.653 (1H, d, J=2.0 Hz), 12.130 (1H, s). LC-MS (ES) 339.2 [M+H]⁺.

### Example 244

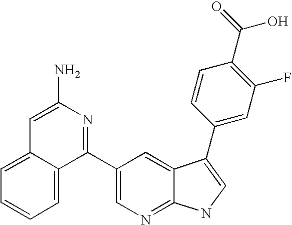

### Preparation of 4-[5-(3-amino-1-isoquinolinyl)-1H-pyrrolo[2,3-b]pyridin-3-yl]-2-fluorobenzoic acid

### a) Preparation of 5-(4,4,5,5-tetramethyl-1,3,2-dioxaborolan-2-yl)-1H-pyrrolo[2,3-b]pyridine

[1,1′-Bis(diphenylphosphino)ferrocene]dichloropalladium(II) complex with dichloromethane (0.031 g, 0.038 mmol) was added to a suspension of 1H-pyrrolo[2,3]pyridine (0.25 g, 1.27 mmol), bis(pinacolato)diboron (0.52 g, 2.03 mmol), and potassium acetate (0.5 g, 5.08 mmol) in dioxane (13 mL). The reaction mixture was heated in the microwave at 150° C. for 20 min, and then filtered through a pad of Celite. The filtrate was partitioned between water and ethyl acetate, and the organic layer was dried over sodium sulfate and concentrated. The residue was purified by flash column chromatography with 20% ethyl acetate in hexane grading to 50% ethyl acetate in hexane to give the pure product (0.134 g, 43%) as a white solid. ¹H-NMR (CDCl₃) δ: 1.406 (12H, s), 6.454 (1H, d, J=3.6 Hz), 7.371 (1H, d, J=3.6 Hz), 8.440 (1H, d, J=1.2 Hz), 8.735 (1H, d, J=1.2 Hz), 10.820 (1H, s). MS (ES) m/e 245.0 [M+H]⁺.

### b) Preparation of 1,1-dimethylethyl [1-(1H-pyrrolo[2,3-b]pyridin-5-yl)-3-isoquinolinyl]carbamate

[1,1′-Bis(diphenylphosphino)ferrocene]dichloropalladium(II) complex with dichloromethane (200 mg, 0.24 mmol) was added to a suspension of 5-(4,4,5,5-tetramethyl-1,3,2-dioxaborolan-2-yl)-1H-pyrrolo[2,3-b](0.680 g, 2.79 mmol), 1,1-dimethylethyl(1-bromo-3-isoquinolinyl)carbamate (0.818 g, 2.53 mmol), and potassium carbonate (1.4 g, 10.13 mmol) in 2.5:1 dioxane/water (28 mL). The reaction mixture was stirred at 95° C. for 1 h. After cooling to room temperature, the mixture was filtered through a pad of Celite. The filtrate was partitioned between water and ethyl acetate, and the organic layer was dried over sodium sulfate and concentrated. The crude product was washed several times with ethyl acetate to give the pure product (0.52 g, 57%) as a light yellow solid. ¹H-NMR (DMSO-d₆) δ: 1.519 (9H, s), 6.590 (1H, t, J=1.6 Hz), 7.460 (1H, m), 7.597 (1H, t, J=4.0 Hz), 7.710 (1H, m), 7.947 (1H, m), 8.156 (1H, s), 8.259 (1H, d, J=2.0 Hz), 8.508 (1H, d, J=2 Hz), 9.862 (1H, s), 11.880 (1H, s). LC-MS (ES) 361.0 [M+H]⁺.

### c) Preparation of 1,1-dimethylethyl [1-(3-bromo-1H-pyrrolo[2,3-b]pyridin-5-yl)-3-isoquinolinyl]carbamate

N-bromosuccinimide (0.295 g, 1.66 mmol) in tetrahydrofuran (4 mL) was added drop wise to a solution of 1,1-dimethylethyl [1-(1H-pyrrolo[2,3-b]pyridin-5-yl)-3-isoquinolinyl]carbamate (0.520 g, 1.44 mmol) in tetrahydrofuran (10 mL). The reaction mixture was stirred at room temperature for 45 min, and then partitioned between water and ethyl acetate. The organic layer was washed with 1M NaOH (10 mL), dried over sodium sulfate, and concentrated. The resulting solid was washed several times with ethyl acetate to give the pure product (0.45 g, 79%) as a light yellow solid. ¹H-NMR (DMSO-d₆) δ: 1.180 (9H, s), 7.50 (1H, m), 7.750 (1H, m), 7.87 (1H, s), 7.97 (2H, m), 8.12 (1H, s), 8.18 (1H, s), 8.60 (1H, s), 9.92 (1H, s), 12.35 (1H, s). LC-MS (ES) 441.4 [M+H]⁺.

### d) Preparation of 1,1-dimethylethyl 3-bromo-5-[3-({[(1,1-dimethylethyl)oxy]carbonyl}amino)-1-isoquinolinyl]-1H-pyrrolo[2,3-b]pyridine-1-carboxylate

Di-tert-butyldicarbonate (0.28 b, 1.3 mmol) was added to a solution of 1,1-dimethylethyl [1-(3-bromo-1H-pyrrolo[2,3-b]pyridin-5-yl)-3-isoquinolinyl]carbamate (0.48 g, 1.1 mmol) and 4-dimethylaminopyridine (0.013 g, 0.11 mmol) in tetrahydrofuran (10 mL). The reaction was maintained at room temperature for 30 min. Solvent was removed, and the residue was partitioned between ethyl acetate and water. The layers were separated and the organic layer was washed with brine, dried over sodium sulfate, and concentrated to give the product as a light green foam (0.5 g, 84%). ¹H-NMR (DMSO-d₆) δ: 1.52 (9H, s), 1.66 (9H, s), 7. 50 (1H, m), 7.72 (1H, m), 7.97 (2H, m), 8.18 (1H, s), 8.23 (1H, m), 8.77 (1H, d, J=2.0 Hz), 9.97 (1H, s). LC-MS (ES) 541.4 [M+H]⁺.

### e) Preparation of 4-[5-(3-amino-1-isoquinolinyl)-1H-pyrrolo[2,3-b]pyridin-3-yl]-2-fluorobenzoic acid

[1,1′-Bis(diphenylphosphino)ferrocene]dichloropalladium(II) complex with dichloromethane (15 mg, 0.02 mmol) was added to a suspension of 1,1-dimethylethyl 3-bromo-5-[3-({[(1,1-dimethylethyl)oxy]carbonyl}amino)-1-isoquinolinyl]-1H-pyrrolo[2,3-b]pyridine-1-carboxylate (0.10 g, 0.185 mmol), 4-(dihydroxyboranyl)-2-fluorobenzoic acid (0.05 g, 0.28 mmol) and potassium carbonate (0.10 g, 0.74 mmol) in 3:1 dioxane/water (2 mL). The reaction flask was equipped with a water-cooled condenser, and the reaction mixture was heated to reflux under a nitrogen atmosphere. After three hours, the mixture was cooled to room temperature and acidified with concentrated HCl. The mixture was then filtered through a pad of Celite, and the filtrate was partitioned between ethyl acetate (10 mL) and water (10 mL). The layers were separated, and the aqueous layer was further extracted with ethyl acetate. The combined organics were dried over sodium sulfate and concentrated. The residue was then treated with 4M HCl/dioxane (0.2 mL) at room temperature for 30 min. The reaction mixture was concentrated and purified via HPLC to give the product (15 mg, 20%) as a yellow solid. ¹H-NMR (DMSO-d₆) δ: 6.89 (1H, s), 7.21 (1H, m), 7.57 (1H, m), 7.75 (4H, m), 7.95 (1H, m), 8.33 (1H, d, J=2.4 Hz), 8.57 (1H, d, J=2.0 Hz), 8.68 (1H, s), 12.52 (1H, s), 13.09 (1H, s). LC-MS (ES) 399.0 [M+H]⁺.

### Example 245

### FP assay of SGK1

The SGK1 assay used fluorescence polarization to monitor the binding of test compounds to the enzyme. An assay mixture, containing 1 nM competent SGK1 enzyme, 0.5 nM ligand A, 1 mM CHAPS, 1 mM DTT, and 10 mM MgCl₂ in 50 mM HEPES, was prepared and allowed to incubate for 15 minutes. After incubation, 20 ul or 40 ul of the assay mixture solution was added to plates containing 0.1 ul or 1 ul of test compound/well. (The amount of enzyme-ligand assay solution was scaled relative to the volume of compound plated keeping the % DMSO at or below 2.5%). The plates were then centrifuged at 1500 rev/min for 1 min after 1 ul of 120 uM ligand B was added to the low control wells. Compound plates were incubated for 2 hours at room temperature and then counted on a LJL Acquest (Molecular Devices). The plates were read in fluorescence polarization mode with excitation at 485 nm and emission at 530 nm using a 505 nm dichromic cut off filter.

### Preparation of Ligand B

### N¹-{3-[5-amino-6-(1-ethyl-1H-imidazo[4,5-c]pyridin-2-yl)-2-pyrazinyl]phenyl}glycinamide (ligand B)

### Preparation of Ethyl-(3-nitropyridin-4-yl)amine

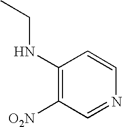

4-Methoxy-3-nitropyridine hydrochloride (11.2 g, 58.9 mmol) in ethanol (75 ml) was treated with a 70% solution of ethylamine in water (64 ml) and heated under reflux for 2 hours. After cooling to room temperature, the solvent was removed in vacuo and the residue dissolved in ethyl acetate and water. The mixture was extracted (×3) with ethyl acetate, washed with water and saturated aqueous sodium chloride solution before drying over magnesium sulfate. Evaporation of the solvent afforded the title compound (11.7 g, 96%).

¹H NMR (400 MHz, DMSO-D6) 8 ppm 1.18 (t, J=7.14 Hz, 3H), 3.41 (m, 2H), 6.98 (d, J=6.32 Hz, 1H), 8.24 (d, J=6.32 Hz, 1H), 8.39 (br, 1H), 9.00 (s, 1H). MS m/z 168 (M+1)⁺.

### Preparation of N⁴-ethylpyridine-3,4-diamine

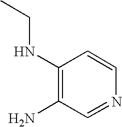

Ethyl-(3-nitropyridin-4-yl)amine (8.7 g, 52.0 mmol) in ethanol (150 ml) was hydrogenated for 18 hours in the presence of 10% palladium on carbon. After filtration of the catalyst through celite, the filtrate was concentrated in vacuo to afford the title compound (6.7 g, 94%).

¹H NMR (400 MHz, DMSO-D6) δ ppm 1.19 (m, 3H), 3.09 (m, 2H), 4.53 (br, 2H), 5.21 (br, 1H), 6.31 (d, J=5.22 Hz, 1H), 7.57 (d, J=5.36 Hz, 1H), 7.62 (s, 1H). MS (ES+) m/e 138 [M+H]⁺.

### Preparation of 3-aminopyrazine-2-carbaldehyde

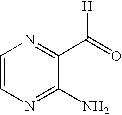

Methyl-3-aminopyrazine-2-carboxylate (11 g) was dissolved in THF and cooled to −78° C. Diisobutylaluminum hydride (1M in hexanes, 250 mL) was added, and the reaction stirred at −78° C. for 4 hours. The reaction was then warmed to 0° C. for one hour before being quenched slowly by addition of 1M hydrochloric acid. Ethyl acetate was added and the layers separated. The organic layer was dried over magnesium sulfate, filtered and concentrated. The residue was triturated in hexanes to afford title compound (3.0 g, 34%).

¹H NMR (400 MHz, DMSO-D6) 8 ppm 7.73 (br, 2H), 8.07 (d, J=2.25 Hz, 1H), 8.36 (d, J=2.11 Hz, 1H), 9.95 (s, 1H).

### Preparation of: 3-(1-ethyl-1H-imidazo[4,5-c]pyridin-2-yl)pyrazin-2-amine

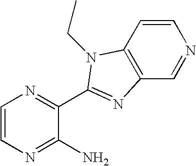

3-aminopyrazine-2-carbaldehyde (0.30 g, 2.4 mmol), N⁴-ethylpyridine-3,4-diamine and sodium hydrogensulfite (0.30 g) were combined in 3 mL of dimethylacetamide and heated to 200° C. in a SmithSynthesizer microwave for 10 minutes. The reaction mixture was partitioned between ethyl acetate and water. The reaction was extracted with ethyl acetate (×3), and the combined organic layers were washed with water and saturated aqueous sodium chloride solution, and dried over magnesium sulfate. After filtration, the organics were concentrated in vacuo. Trituration with diethyl ether affords the title compound as tan solids (0.46 g, 80%).

¹H NMR (400 MHz, DMSO-D6) 3 ppm 1.37 (t, J=7.14 Hz, 3H), 4.82 (q, J=6.87 Hz, 2H), 7.76 (d, J=6.18 Hz, 1H), 8.01 (d, J=2.06 Hz, 1H) 8.07 (br, 2H), 8.17 (d, J=2.34 Hz, 1H), 8.42 (d, J=5.50, 1H) 9.05 (s, 1H) MS m/z 241 (M+1)⁺.

### Preparation of 5-bromo-3-(1-ethyl-1H-imidazo[4,5-c]pyridin-2-yl)pyrazin-2-amine

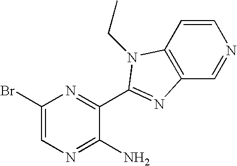

To a solution of 3-(1-ethyl-1H-imidazo[4,5-c]pyridin-2-yl)pyrazin-2-amine (0.45 g, 1.8 mmol) in THF is added N-bromosuccinimide (0.37 g, 2.1 mmol). The reaction is stirred for 4 hours at ambient temperature. Sodium sulfite (excess) was added and stirred vigorously for 30 minutes. The mixture was then concentrated to dryness and treated with water. After sonication, the solids were collected by filtration. Rinsing (×2) with diethyl ether afforded 5-bromo-3-(1-ethyl-1H-imidazo[4,5-c]pyridin-2-yl)pyrazin-2-amine (0.47 g, 79%) as tan solids.

¹H NMR (400 MHz, DMSO-D6) 5 ppm 1.45 (t, J=7.16 Hz, 3H) 4.79 (q, J=7.02 Hz, 2H) 7.82 (dd, J=5.62 0.84 Hz, 1H), 8.38 (s, 1H), 8.49 (d, J=5.76 Hz, 1H) 9.12 (d, J=0.56 Hz, 1H) MS m/z 318/320 (M+1)⁺.

### Preparation of 2-(1,3-dioxo-1,3-dihydro-2H-isoindol-2-yl)-N-[3-(4,4,5,5-tetramethyl-1,3,2-dioxaborolan-2-yl)phenyl]acetamide

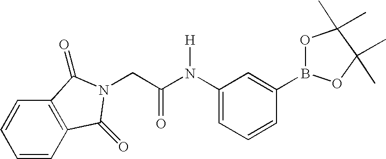

A solution of 3-(4,4,5,5-tetramethyl-1,3,2-dioxaborolan-2-yl)aniline (6 g, 27.4 mmol), N-phthaloylglycine (5.5 g, 27 mmol) and HOBT (6.2 g, 32.4 mmol) in DMF (85 mL) was treated with the water soluble carbodiimide EDC (5.1 g, 37.8 mmol) and stirred at ambient temperature for 18 hours. The reaction mixture was poured into ice water (500 mL). The precipitated solids were collected by filtration, washed with water (3×200 mL) and Et₂O (3×200 mL) and dried to give the title compound (6.6 g, 60%).

¹H NMR (300 MHz, DMSO-D6) 8 ppm 10.4 (s, 1H), 8.0 (m, 5H), 7.7 (d, 1H, J=7.3 Hz), 7.4 (m, 2H), 4.5 (s, 2H), 1.3 (m, 12H).

### Preparation of Compound B: N¹-{3-[5-amino-6-(1-ethyl-1H-imidazo[4,5-c]pyridin-2-yl)-2-pyrazinyl]phenyl}glycinamide

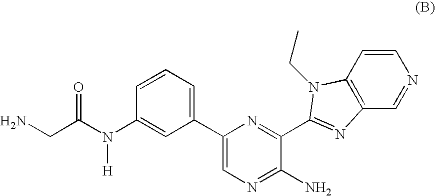

5-bromo-3-(1-ethyl-1H-imidazo[4,5-c]pyridin-2-yl)pyrazin-2-amine (preparation e above, 200 mg, 0.62 mmol), 3-(4,4,5,5-tetramethyl-1,3,2-dioxaborolan-2-yl)aniline (preparation f above, 378 mg, 0.93 mmol) and bistriphenylphosphine palladium dichloride (22 mg, 0.03 mmol) were combined in a 2-5 mL microwave vial from Personal Chemistry. DMF (2 mL) and EtOH (1 mL) were added, then the suspension was treated with aqueous Na₂CO₃ (0.62 mL of a 2M aqueous solution, 1.24 mmol). The vial was sealed and heated in a SmithSynthesizer microwave at 160° C. for 1200 seconds. The reaction mixture was cooled, and the residue was dissolved in MeOH and filtered through celite. The solvent was evaporated and the residue was taken up in EtOH (50 mL), treated with hydrazine hydrate (0.18 mL, 3.72 mmol), and heated at 70° C. for 12 hours. The reaction mixture was concentrated, and the residue purified by reverse phase HPLC (gradient elution, 0 to 100% CH₃CN with 0.01% TFA). The appropriate fractions were combined and the solvent evaporated to give yellow solids. These solids were suspended in EtOH and the solvent evaporated (3×) to afford the title compound (100 mg, 41%).

¹H NMR (300 MHz, DMSO-D6) δ ppm 10.7 (s, 1H), 9.5 (s, 1H), 8.9 (s, 1H), 8.7 (d, 1H, J=6.4), 8.3 (m, 6H), 7.9 (m, 1H), 7.8 (m, 1H), 7.6 (s, 1H), 7.5 (m, 2H), 5.1 (m, 2H), 3.9 (broad, 2H), 1.6 (t, 3H, J=7.1).

### Preparation of Compound A: (5-Rodamine Green conjugate of Compound B) N¹-{3-[5-amino-6-(1-ethyl-1H-imidazo[4,5-c]pyridin-2-yl)-2-pyrazinyl]phenyl}glycinamide

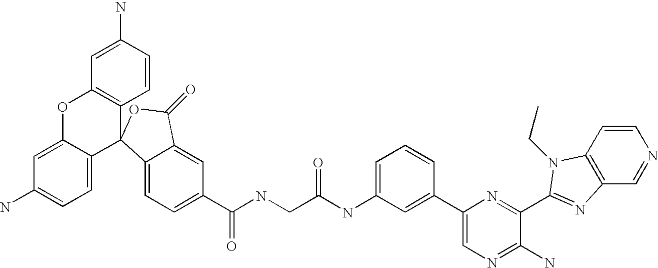

Compound A (5.88 mg, 11.7 umol) was dissolved in dimethylformamide (200 ul), diluted with water (100 ul) and solid sodium carbonate added to saturation. Rhodamine Green hydroxysuccinimide ester (Molecular Probes, 5,6-mixed isomers, 5 mg, 9.8 umol) was added as a solution in dimethylformamide (300 ul total) and the mixture agitated gently for 16 h. Analysis by analytical HPLC (Spherisorb ODS2, 20 to 50% B/1 h. where A=0.1% Trifluoroacetic acid in water and B=0.1% Trifluoroacetic acid in 90% acetonitrile/10% water) showed removal of the active ester. ES LC/MS identified both isomers, mass observed 745.53. Calculated for C₄₁H₃₂N₁₀O₅, M+H⁺=745.78.

The reaction mixture was evaporated to dryness in vacuo, the residue re-dissolved in acetic acid/acetonitrile/water (1/5/4,1 ml) and filtered. The solution was purified by preparative reverse phase HPLC (Phenomenex Jupiter C18, 10 u, 300 A, 250×21.2 mm) using a 20 to 50% B gradient over 1 h, detection at 214 nm. Fractions were analysed by ES LC/MS and reverse phase HPLC. The slower eluting 5-isomer (Compound B) was collected (>95% purity) and evaporated to dryness (3.0 mg, 34%). ES LC/MS M+2H/2⁺ (obs): 373.47, calc. 373.40, M+H⁺ obs. 745.53 (weak), Calc. 745.78.

The examples of this invention were tested in the FP assay protocol. All of the compounds displayed SGK-1 inhibitory activity with IC_{50's} below 1.5 uM.

It is to be understood that the invention is not limited to the embodiments illustrated hereinabove and the right is reserved to the illustrated embodiments an all modifications coming within the scope of the following claims.

What is claimed is:

1. A method of treating a disorder in a mammal, said disorder being mediated by SGK activity, comprising administering to said mammal a therapeutically effective amount of a compound of formula (I), 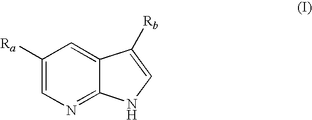
wherein: 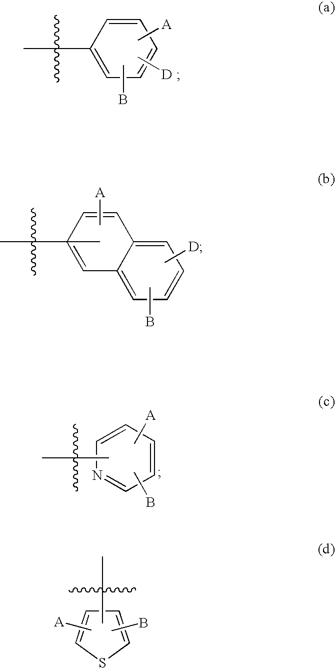
Rₐ is 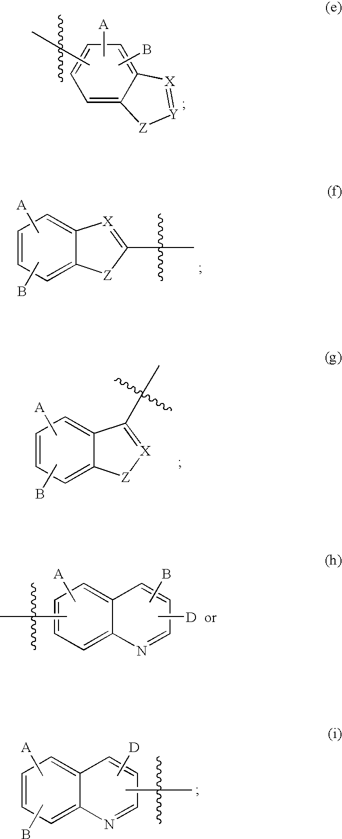
wherein:
A, B, D, and R^{1a} are each independently hydrogen; OR; CN; halogen; CO₂R; CONR₁R₂; NR₁R₂; NR₃R₄; aryl; heteroaryl; (C₁₋₃)alkyl-NR₁R₂; (C₁₋₆)alkyl, or (C₁₋₆)haloalkyl;
X and Y are each independently CR^{1a} or N;
Z is NR, O or S; 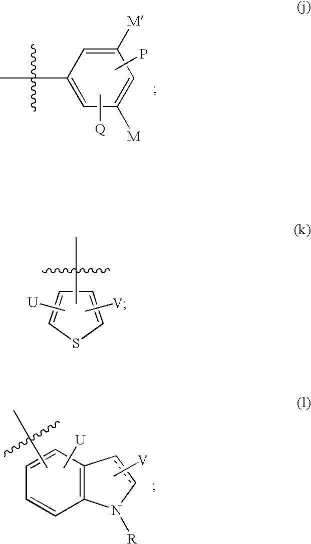
R_{b} is 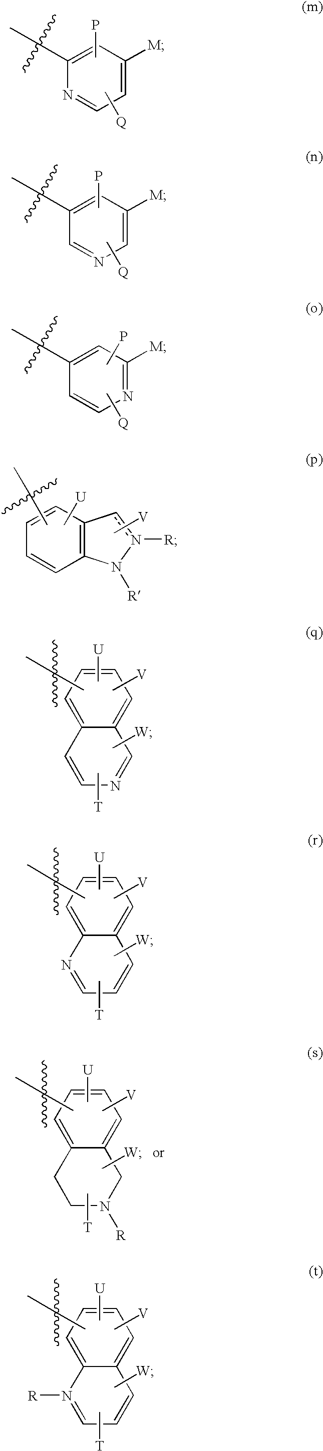
M is independently hydrogen; (C₁₋₃)alkyl-NR₁R₂; (C₁₋₃)alkyl-OR; halogen; CO₂R; OR; NR₁R₂; (C₁.)alkyl-NR₃R₄; CONR₁R₂; (C₁₋₆)alkyl-CONR₁R₂; CHO; (C₁₋₆)alkylCO₂R; (C₁₋₆)alkyl; or NR₃R₄;
M′ is independently hydrogen; (C₁₋₃)alkyl-NR₁R₂; (C₁₋₃)alkyl-OR; halogen; CO₂R; OR; NR₁R₂; (C₁₋₃)alkyl-NR₃R₄; CONR₁R₂; (C₁₋₆)alkyl-CONR₁R₂; CHO; (C₁₋₆)alkylCO₂R; NR₃R₄; (C₁₋₆)alkyl; or phenyl;
P, Q, T, U, V and W are each independently hydrogen; halogen; (C₁₋₆)alkyl; (C₁₋₃)alkylOR; (C₁₋₆)haloalkyl; CO₂R; CHO; (C₁₋₆)alkyl-CO₂R; (C₁₋₃)alkyl-NR₁R₂; OR; NR₁R₂; NR₃R₄; CONR₁R₂; (C₁₋₆)alkyl-CONR₁R₂; aryl; or heteroaryl;
R and R′ are independently at each occurrence hydrogen; (C₁₋₃)alkylaryl; (C₁₋₃)alkylheteroaryl; (C₁₋₆)alkyl; or (C₁₋₆)haloalkyl;
R₁ and R₂ are independently at each occurrence hydrogen; (C₁₋₆)alkyl; (C₁₋₃)alkylNRR′; (C₁₋₃)alkylOR; (C₁₋₆)cyanoalkyl; NRR′; (C₁₋₃)alkylaryl; (C₁₋₃)alkylheteroaryl; (C₁₋₆)haloalkyl; or together with the nitrogen that they are attached form a 4, 5, 6, or 7 member non-aromatic ring, said ring optionally containing up to 2 additional heteroatoms selected from the group consisting of NR; O; or S(O)ₙ; and said ring is unsubstituted or substituted with from 1-3 substituents selected from the group consisting of halogen; (C₁₋₆)alkyl; OR; NRR′; CN; halogen; (C₁₋₆)haloalkyl; phenyl; heteroaryl and heterocyclyl;
n is independently at each occurrence 0, 1 or 2;
R₃ is independently at each occurrence hydrogen; (C₁₋₆)alkyl; or (C₁₋₆)haloalkyl;
R₄ is C(═O)(C₁₋₆)alkyl; C(═O)(C₁₋₃)alkyl-NRR′; or C(═O)—(C₁₋₃)alkylaryl wherein said aryl is unsubstituted or substituted with 1-3 substituents selected from the group consisting of halogen, (C₁₋₃)alkyl and (C₁₋₃)alkoxy); C(═O)—(C₁₋₃)alkylheteroaryl; C(═O)phenyl wherein the phenyl group is unsubstituted or substituted with 1-3 substituents selected from the group consisting of halogen, (C₁₋₆)alkyl and OR;
or a pharmaceutically acceptable salt or solvate thereof.

2. The method of treatment of claim 1 wherein said disorder is a proliferative response to an insult or injury.

3. The method of treatment of claim 1 wherein said disorder is undesired water retention.

4. The method of claim 1 wherein said disorder is renal disease.

5. The method of claim 1 wherein said disorder is cardiovascular disease.

6. A compound of formula (II), 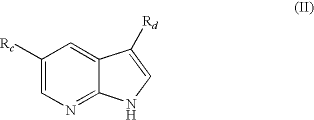
wherein: 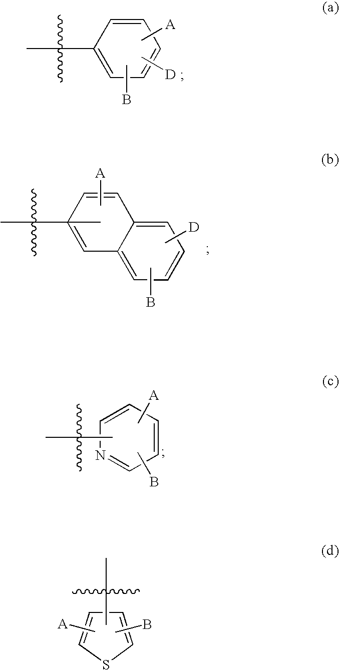
R_{c} is 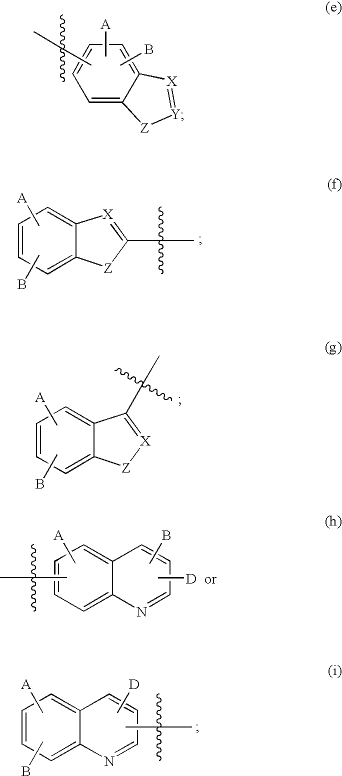
wherein:
A, B, D, and R^{1a} are each independently hydrogen; OR; CN; halogen; CO₂R; CONR₁R₂; NR₁R₂; NR₃R₄; S(O)ₙ(C₁₋₆)alkyl wherein said alkyl is unsubstituted or substituted with 1-3 substituents selected from the group consisting of halogen; OR; NRR′; and CN; aryl; heteroaryl; (C₁₋₃)alkyl-NR₁R₂; (C₁₋₆)alkyl; or (C₁₋₆)haloalkyl;
X and Y are each independently CR^{1a} or N;
Z is NR, O or S; 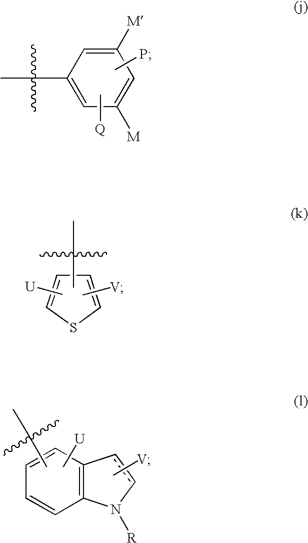
R_{d} is 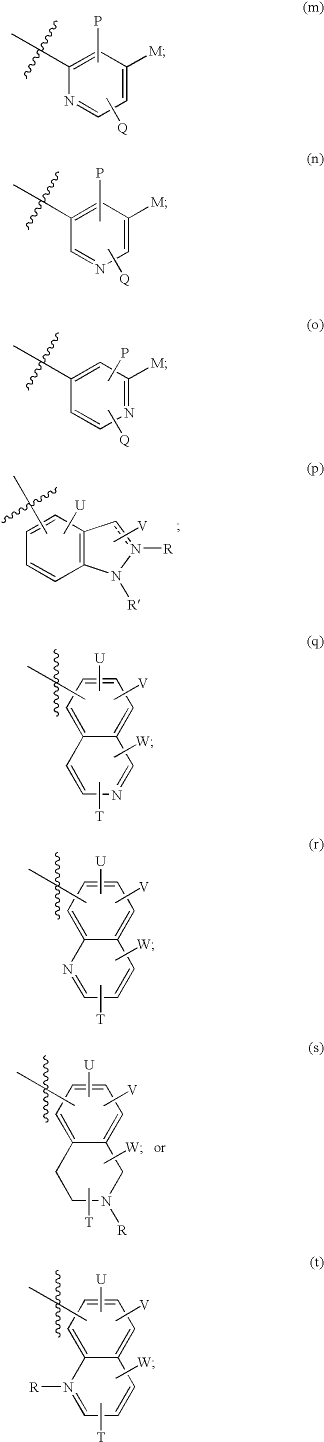
M is independently hydrogen; (C₁₋₃)alkyl-NR₁R₂; (C₁₋₃)alkyl-OR; halogen; CO₂R; OR; S(O)ₙ(C₁₋₆)alkyl wherein said alkyl is unsubstituted or substituted with 1-3 substituents selected from the group consisting of halogen; OR; NRR′; and CN); NR₁R₂; (C₁₋₃)alkyl-NR₃R₄; SO₂NR₁R₂; (C₁₋₃)alkyl-SO₂NR₁R₂; CONR₁R₂; (C₁₋₆)alkyl-CONR₁R₂; CHO; (C₁₋₆)alkylCO₂R; (C₁₋₆)alkyl; and NR₃R₄;
M′ is independently hydrogen; (C₁₋₃)alkyl-NR₁R₂; (C₁₋₃)alkyl-OR; halogen; CO₂R; OR; S(O)ₙ(C₁₋₆)alkyl wherein said alkyl is unsubstituted or substituted with 1-3 substituents selected from the group consisting of halogen; OR; NRR′; and CN); NR₁R₂; (C₁₋₃)alkyl-NR₃R₄; SO₂NR₁R₂; (C₁₋₃)alkyl-SO₂NR₁R₂; CONR₁R₂; (C₁₋₆)alkyl-CONR₁R₂; CHO; (C₁₋₆)alkylCO₂R; NR₃R₄; (C₁₋₆)alkyl; and phenyl;
P and Q are each independently hydrogen; halogen; (C₁₋₆)alkyl; (C₁₋₃)alkylOR; (C₁₋₆)haloalkyl; CO₂R; CHO; S(O)ₙ(C₁₋₆)alkyl wherein said alkyl is unsubstituted or substituted with 1-3 substituents selected from the group consisting of halogen; OR; NRR′; and CN); (C₁₋₆)alkyl-CO₂R; (C₁₋₃)alkyl-NR₁R₂; (C₁₋₃)alkyl-NR₃R₄; OR; NR₁R₂; NR₃R₄; CONR₁R₂; (C₁₋₆)alkyl-CONR₁R₂; SO₂NR₁R₂; (C₁₋₃)alkyl-SO₂NR₁R₂; aryl; and heteroaryl;
T, U, V and W are each independently hydrogen; halogen; (C₁₋₆)alkyl; (C₁₋₃)alkylOR; (C₁₋₆)haloalkyl; CO₂R; CHO; S(O)ₙ(C₁₋₆)alkyl wherein said alkyl is unsubstituted or substituted with 1-3 substituents selected from the group consisting of halogen; OR; NRR′; and CN); (C₁₋₆)alkyl-CO₂R; (C₁₋₃)alkyl-NR₁R₂; (C₁₋₃)alkyl-NR₃R₄; OR; NR₁R₂; NR₃R₄; CONR₁R₂; (C₁₋₆)alkyl-CONR₁R₂; SO₂NR₁R₂; (C₁₋₃)alkyl-SO₂NR₁R₂; aryl; and heteroaryl;
R and R′ are independently at each occurrence hydrogen; (C₁₋₃)alkylaryl; (C₁₋₃)alkylheteroaryl; (C₁₋₆)alkyl; or (C₁₋₆)haloalkyl;
R₁ and R₂ are independently at each occurrence hydrogen; (C₁₋₆)alkyl; (C₁₋₃)alkylNRR′; (C₁₋₃)alkylOR; (C₁₋₆)cyanoalkyl; NRR′; (C₁₋₃)alkylaryl, (C₁₋₃)alkylheteroaryl; (C₁₋₆)haloalkyl; or together with the nitrogen that they are attached form a 4, 5, 6, or 7 member non-aromatic ring, said ring optionally containing up to 2 additional heteroatoms selected from the group consisting of NR; O; or S(O)ₙ; and said ring being unsubstituted or substituted with from 1-3 substituents selected from the group consisting of halogen; (C₁₋₆)alkyl; OR; NRR′; CN; halogen; (C₁₋₆)haloalkyl; phenyl; heteroaryl or heterocyclyl;
n is independently at each occurrence 0, 1 or 2;
R₃ is independently at each occurrence hydrogen; (C₁₋₆)alkyl; or (C₁₋₆)haloalkyl;
R₄ is C(═O)(C₁₋₆)alkyl; C(═O)(C₁₋₃)alkyl-NRR′; C(═O)—(C₁₋₃)alkylaryl wherein said aryl is unsubstituted or substituted with 1-3 substituents selected from halogen, (C₁₋₃)alkyl and C₁₋₃alkoxy); C(═O)—(C₁₋₃)alkylheteroaryl; S(O)ₙ(C₁₋₆)alkyl; SO₂NR₁R₂; C(═O)-phenyl wherein the phenyl group is unsubstituted or substituted with 1-3 substituents selected from the group consisting of halogen, (C₁₋₆)alkyl and OR;
or a pharmaceutically acceptable salt or solvate thereof; and
provided at least one of M, P, and Q is (C₁₋₆)alkyl-CO₂R or (C₁₋₆)alkyl-CONR₁R₂; and
provided at least one of U and V, or at least one of U, V, T and W is (C₁₋₆)alkyl-CO₂R or (C₁₋₆)alkyl-CONR₁R₂.

7. A compound according to claim 6 wherein R_{c} is (a), (b), (c) or (d).

8. A compound according to claim 7 wherein R_{c} is (a).

9. A compound according to claim 7 wherein R_{d} is (j), (l), (m), (n) or (o).

10. A compound according to claim 9 wherein R_{d} is (j).

11. A compound according to claim 10 wherein at least one of m, p, or q is (C₁₋₆)alkylCO₂H.

12. A compound according to claim 10 wherein R_{c} is (a) or (b).

13. A compound according to claim 12 wherein at least one of m, p, or q is (C₁₋₆)alkylCO₂H.

14. A compound selected from the group consisting of:
a) 4-(5-phenyl-1H-pyrrolo[2,3-b]pyridin-3-yl)-benzoic acid;
b) {3-[5-(2-naphthyl)-1H-pyrrolo[2,3-b]pyridin-3-yl]benzyl}amine;
c) 4-[5-(2-naphthalenyl)-1H-pyrrolo[2,3-b]pyridin-3-yl]benzoic acid;
d) {4-[5-(2-naphthalenyl)-1H-pyrrolo[2,3-b]pyridin-3-yl]phenyl}acetic acid;
e) 3-{4-[5-(2-naphthalenyl)-1H-pyrrolo[2,3-b]pyridin-3-yl]phenyl}propanoic acid;
f) {3-[5-(2-naphthyl)-1H-pyrrolo[2,3-b]pyridin-3-yl]phenyl}methanol;
g) 4-{5-[3-(methyloxy)phenyl]-1H-pyrrolo[2,3-b]pyridin-3-yl}benzoic acid;
h) 3-(4-{5-[3-(methyloxy)phenyl]-1H-pyrrolo[2,3-b]pyridin-3-yl}phenyl)propanoic acid;
i) 3-{3-[4-(aminomethyl)phenyl]-1H-pyrrolo[2,3-b]pyridin-5-yl}benzonitrile;
j) 4-{5-[3-(aminocarbonyl)phenyl]-1H-pyrrolo[2,3-b]pyridin-3-yl}benzoic acid;
k) 4-[5-(3-cyanophenyl)-1H-pyrrolo[2,3-b]pyridin-3-yl]benzoic acid;
l) 4-{5-[6-(methyloxy)-3-pyridinyl]-1H-pyrrolo[2,3-b]pyridin-3-yl}benzoic acid;
m) 3-{3-[3-(aminomethyl)phenyl]-1-pyrrolo[2,3-b]pyridin-5-yl}benzonitrile;
n) 4-[5-(1-naphthalenyl)-1H-pyrrolo[2,3-b]pyridin-3-yl]benzoic acid;
o) 2-fluoro-4-[5-(1-naphthalenyl)-1H-pyrrolo[2,3-b]pyridin-3-yl]benzoic acid;
p) 3-amino-5-[5-(1-naphthalenyl)-1H-pyrrolo[2,3-b]pyridin-3-yl]benzoic acid;
q) 3-{4-[5-(1-naphthalenyl)-1H-pyrrolo[2,3-b]pyridin-3-yl]phenyl}propanoic acid;
r) 3-(4-{5-[3-(aminocarbonyl)phenyl]-1H-pyrrolo[2,3-b]pyridin-3-yl}phenyl)propanoic acid;
s) 3-{4-[5-(3-cyanophenyl)-1H-pyrrolo[2,3-b]pyridin-3-yl]phenyl}propanoic acid;
t) 3-(4-{5-[6-(methyloxy)-3-pyridinyl]-1H-pyrrolo[2,3-b]pyridin-3-yl}phenyl)propanoic acid;
u) {4-[5-(1-naphthalenyl)-1H-pyrrolo[2,3-b]pyridin-3-yl]phenyl}acetic acid;
v) (4-{5-[3-(aminocarbonyl)phenyl]-1H-pyrrolo[2,3-b]pyridin-3-yl}phenyl)acetic acid;
w) {4-[5-(3-cyanophenyl)-1H-pyrrolo[2,3-b]pyridin-3-yl]phenyl}acetic acid;
x) (4-{5-[6-(methyloxy)-3-pyridinyl]-1H-pyrrolo[2,3-b]pyridin-3-yl}phenyl)acetic acid;
y) 3-{4-[5-(3-Methanesulfonylamino-phenyl)-1H-pyrrolo[2,3-b]pyridin-3-yl}-phenyl]-propionic acid;
z) {4-[5-(3-Methanesulfonylamino-phenyl)-1H-pyrrolo[2,3-b]pyridin-3-yl]-phenyl}-acetic acid;
aa) 3-{4-[5-(3-Methanesulfonyl-phenyl)-1H-pyrrolo[2,3-b]pyridin-3-yl]-phenyl}-propionic acid;
ab) {4-[5-(3-Methanesulfonyl-phenyl)-1H-pyrrolo[2,3-b]pyridin-3-yl]-phenyl}-acetic acid;
ac) 3-[3-fluoro-4-(methyloxy)phenyl]-5-phenyl-1H-pyrrolo[2,3-b]pyridine;
ad) 5-phenyl-3-pyridin-4-yl-1H-pyrrolo[2,3-b]pyridine;
ae) 3-(5-phenyl-1H-pyrrolo[2,3-b]pyridin-3-yl)-benzoic acid;
af) N-[3-(5-phenyl-1H-pyrrolo[2,3-b]pyridin-3-yl)-phenyl]-acetamide;
ag) 4-(5-phenyl-1H-pyrrolo[2,3-b]pyridin-3-yl)-phenylamine;
ah) 4-(5-phenyl-1H-pyrrolo[2,3-b]pyridin-3-yl)-phenol;
ai) 4-(5-phenyl-1H-pyrrolo[2,3-b]pyridin-3-yl)-benzylamine;
aj) 3-(5-phenyl-1H-pyrrolo[2,3-b]pyridin-3-yl)-phenol;
ak) 5-(3,4-dimethoxyphenyl)-3-pyridin-4-yl-1H-pyrrolo[2,3-b]pyridine;
al) 4-[5-(3,4-dimethoxyphenyl)-1H-pyrrolo[2,3-b]pyridin-yl]-phenol;
am) 4-[5-(3,4-dimethoxyphenyl)-1H-pyrrolo[2,3-b]pyridin-yl]-phenylamine;
an) 4-[5-(3,4-dimethoxyphenyl)-1H-pyrrolo[2,3-b]pyridin-3-yl]-benzoic acid;
ao) 4-[5-(4-chlorophenyl)-1H-pyrrolo[2,3-b]pyridin-3-yl]-benzoic acid;
ap) N-[4-(5-phenyl-1H-pyrrolo[2,3-b]pyridin-3-yl)-phenyl]-acetamide;
aq) 3,5-bis-(4-hydroxyphenyl)-1H-pyrrolo[2,3-b]pyridine;
ar) 3,5-bis-(4-carboxyphenyl)-1H-pyrrolo[2,3-b]pyridine;
as) 4-[5-(4-aminophenyl)-1H-pyrrolo[2,3-b]pyridin-3-yl)-benzylamine;
at) 4-[5(4-aminophenyl)-1H-pyrrolo[2,3-b]pyridin-3-yl)-benzoic acid;
au) 4-[5-(2-fluoro-biphen-4-yl)-1H-pyrrolo[2,3-b]pyridin-3-yl)-benzoic acid;
av) N-[3-(5-thiophen-3-yl-1H-pyrrolo[2,3-b]pyridin-3-yl)-phenyl]-acetamide;
aw) 4-(5-thiophen-3-yl-1H-pyrrolo[2,3-b]pyridin-3-yl)-benzoic acid;
ax) 4-(5-thiophen-3-yl-1H-pyrrolo[2,3-b]pyridin-3-yl)-phenol;
ay) 4-(5-thiophen-3-yl-1H-pyrrolo[2,3-b]pyridin-3-yl)-benzamide;
az) N-[3-(5-pyridin-3-yl-1H-pyrrolo[2,3-b]pyridin-3-yl)-phenyl]-acetamide;
ba) 4-(5-pyridin-3-yl-1H-pyrrolo[2,3-b]pyridin-3-yl)-benzoic acid;
bb) 4-(5-pyridin-3-yl-1H-pyrrolo[2,3-b]pyridin-3-yl)-phenol;
bc) 4-(5-thiophen-3-yl-1H-pyrrolo[2,3-b]pyridin-3-yl)-benzylamine;
bd) 3-(1H-indol-5-yl)-5-thiophen-3-yl-1H-pyrrolo[2,3-b]pyridine;
be) N-[4-(5-thiophen-3-yl-1H-pyrrolo[2,3-b]pyridin-3-yl)-phenyl]-acetamide;
bf) 5-(3-pyridinyl)-3-(4-pyridinyl)-1H-indole;
bg) 4-(5-pyridin-3-yl-1H-pyrrolo[2,3-b]pyridin-3-yl)-benzamide;
bh) 4-[3-(2-fluorobiphenyl-4-yl-1H-pyrrolo[2,3-b]pyridin-5-yl]-benzylamine;
bi) 4-(5-pyridin-3-yl-1H-pyrrolo[2,3-b]pyridin-3-yl)-phenylamine;
bj) {3-[5-(4-methanesulfonylphenyl-1H-pyrrolo[2,3-b]pyridin-3-yl]-phenyl}-acetic acid;
bk)-[3-(3-thiophen-3-yl-1H-pyrrolo[2,3-b]pyridin-5-yl)-phenyl]-acetamide;
bl) N-{3-[3-(3-pyridinyl)-1H-pyrrolo[2,3-b]pyridin-5-yl]phenyl}acetamide;
bm) 4-[5-(3-acetylaminophenyl)-1H-pyrrolo[2,3-b]pyridin-3-yl]-benzoic acid;
bn) N-{3-[3-(2,3-difluorophenyl)-1H-pyrrolo[2,3-b]pyridin-5-yl]-phenyl}-acetamide;
bo) N-{3-[3-(4-hydroxyphenyl)-1H-pyrrolo[2,3-b]pyridin-5-yl]-phenyl}-acetamide;
bp) N-{3-[3-(4-aminomethylphenyl)-1H-pyrrolo[2,3-b]pyridin-5-yl]-phenyl}-acetamide;
bq) N-{3-[3-(4-aminophenyl)-1H-pyrrolo[2,3-b]pyridin-5-yl]-phenyl}-acetamide;
br) N-{3-[3-(1H-indol-5-yl)-1H-pyrrolo[2,3-b]pyridin-5-yl]-phenyl}-acetamide;
bs) 4-[3-(2-fluorobiphenyl-4-yl)-1H-pyrrolo[2,3-b]pyridin-5-yl]-benzoic acid;
bt) N-{3-[3-(4-pyridinyl)-1H-pyrrolo[2,3-b]pyridin-5-yl]phenyl}acetamide;
bu) N-{3-[5-(3-fluorophenyl)-1H-pyrrolo[2,3-b]pyridin-3-yl]-phenyl}-acetamide;
by) 4-[5-(3-acetylaminophenyl)-1H-pyrrolo[2,3-b]pyridin-3-yl]-benzamide;
bw) 4-[S(3-fluorophenyl)-1H-pyrrolo[2,3-b]pyridin-3-yl]-benzoic acid;
bx) 4-[S(3-fluorophenyl)-1H-pyrrolo[2,3-b]pyridin-3-yl]-phenylamine;
by) N-{4-[5-(3-fluorophenyl)-1H-pyrrolo[2,3-b]pyridin-3-yl]-phenyl}-acetamide;
bz) 4-[5-(3-fluorophenyl)-1H-pyrrolo[2,3-b]pyridin-3-yl]-benzamide;
ca) 2-chloro-N-[4-(5-phenyl-1H-pyrrolo[2,3-b]pyridin-3-yl)-phenyl]-benzamide;
cb) 2-phenyl-N-[4-(5-phenyl-1H-pyrrolo[2,3-b]pyridin-3-yl)-phenyl]-acetamide;
cc) 2-chloro-N-[3-(5-phenyl-1H-pyrrolo[2,3-b]pyridin-3-yl)-benzyl]1-benzamide;
cd) 2-phenyl-N-[3-(5-phenyl-1H-pyrrolo[2,3-b]pyridin-3-yl)-benzyl]-acetamide;
ce) (4-{5-[3-(methyloxy)phenyl]-1H-pyrrolo[2,3-b]pyridin-3-yl}phenyl)acetic acid;
cf) 3-[4-(5-{4-[(methylsulfonyl)amino]phenyl}-1H-pyrrolo[2,3-b]pyridin-3-yl)phenyl]propanoic acid;
cg) [4-(5-{4-[(methylsulfonyl)amino]phenyl}-1H-pyrrolo[2,3-b]pyridin-3-yl)phenyl]acetic acid;
ch) (4-{5-[3,4,5-tris(methyloxy)phenyl]-1H-pyrrolo[2,3-b]pyridin-3-yl}phenyl)acetic acid;
ci) 3-(4-{5-[3,4,5-tris(methyloxy)phenyl]-1H-pyrrolo[2,3-b]pyridin-3-yl}phenyl)propanoic acid;
cj) {4-[5-(6-quinolinyl)-1H-pyrrolo[2,3-b]pyridin-3-yl]phenyl}acetic acid;
ck) 3-{4-[5-(6-quinolinyl)-1H-pyrrolo[2,3-b]pyridin-3-yl]phenyl}propanoic acid;
cl) {4-[5-(3-quinolinyl)-1H-pyrrolo[2,3-b]pyridin-3-yl]phenyl}acetic acid;
cm) {4-[5-(5-quinolinyl)-1H-pyrrolo[2,3-b]pyridin-3-yl]phenyl}acetic acid;
cn) 3-{4-[5-(5-quinolinyl)-1H-pyrrolo[2,3-b]pyridin-3-yl]phenyl}propanoic acid;
co) 3-(4-{5-[6-(methyloxy)-2-naphthalenyl]-1H-pyrrolo[2,3-b]pyridin-3-yl}phenyl)propanoic acid;
cp) 3-{4-[5-(3,4-dimethylphenyl)-1H-pyrrolo[2,3-b]pyridin-3-yl]phenyl}propanoic acid;
cq) {4-[5-(3,4-dimethylphenyl)-1H-pyrrolo[2,3-b]pyridin-3-yl]phenyl}acetic acid;
cr) {4-[5-(2,3-dimethylphenyl)-1H-pyrrolo[2,3-b]pyridin-3-yl]phenyl}acetic acid;
cs) 3-{4-[5-(2,3-dimethylphenyl)-1H-pyrrolo[2,3-b]pyridin-3-yl]phenyl}propanoic acid;
ct) {4-[5-(2,3-dichlorophenyl)-1H-pyrrolo[2,3-b]pyridin-3-yl]phenyl}acetic acid;
cu) 3-{4-[5-(2,3-dichlorophenyl)-1H-pyrrolo[2,3-b]pyridin-3-yl]phenyl}propanoic acid;
cv) {4-[5-(1-benzothien-3-yl)-1H-pyrrolo[2,3-b]pyridin-3-yl]phenyl}acetic acid;
cw) [(3-{5-[3-(methyloxy)phenyl]-1H-pyrrolo[2,3-b]pyridin-3-yl}phenyl)methyl]amine;
cx) 7-[5-(2-naphthalenyl)-1H-pyrrolo[2,3-b]pyridin-3-yl]-1,2,3,4-tetrahydroisoquinoline;
cy) 2-fluoro-4-[5-(2-naphthalenyl)-1H-pyrrolo[2,3-b]pyridin-3-yl]benzoic acid;
cz) 2-fluoro-4-{5-[3-(methyloxy)phenyl]-1H-pyrrolo[2,3-b]pyridin-3-yl}benzoic acid;
da) 2-methyl-4-{5-[3-(methyloxy)phenyl]-1H-pyrrolo[2,3-b]pyridin-3-yl}benzoic acid;
db) 5-[5-(2-naphthalenyl)-1H-pyrrolo[2,3-b]pyridin-3-yl]-2-thiophenecarbaldehyde;
dc) 5-{5-[3-(methyloxy)phenyl]-1H-pyrrolo[2,3-b]pyridin-3-yl}-2-thiophenecarbaldehyde;
dd) 2-methyl-4-[5-(2-naphthalenyl)-1H-pyrrolo[2,3-b]pyridin-3-yl]benzoic acid;
de) (4-{5-[6-(methyloxy)-2-naphthalenyl]-1H-pyrrolo[2,3-b]pyridin-3-yl}phenyl)acetic acid;
df) 2-fluoro-4-{5-[6-(methyloxy)-2-naphthalenyl]-1H-pyrrolo[2,3-b]pyridin-3-yl}benzoic acid;
dg) 2-fluoro-4-[5-(5-quinolinyl)-1H-pyrrolo[2,3-b]pyridin-3-yl]benzoic acid;
dh) 4-[5-(5-quinolinyl)-1H-pyrrolo[2,3-b]pyridin-3-yl]benzoic acid;
di) 2-methyl-4-[5-(5-quinolinyl)-1H-pyrrolo[2,3-b]pyridin-3-yl]benzoic acid;
dj) 4-[5-(3,4-dimethylphenyl)-1H-pyrrolo[2,3-b]pyridin-3-yl]benzoic acid;
dk) 4-[5-(3,4-dimethylphenyl)-1H-pyrrolo[2,3-b]pyridin-3-yl]-2-fluorobenzoic acid;
dl) 4-[5-(3,4-dimethylphenyl)-1H-pyrrolo[2,3-b]pyridin-3-yl]-2-methylbenzoic acid;
dm) 4-[5-(2,3-dichlorophenyl)-1H-pyrrolo[2,3-b]pyridin-3-yl]benzoic acid;
dn) 4-[5-(2,3-dichlorophenyl)-1H-pyrrolo[2,3-b]pyridin-3-yl]-2-methylbenzoic acid;
do) 4-[5-(1-benzothien-3-yl)-1H-pyrrolo[2,3-b]pyridin-3-yl]benzoic acid;
dp) 4-[5-(1-benzothien-3-yl)-1H-pyrrolo[2,3-b]pyridin-3-yl]-2-fluorobenzoic acid;
dq) 4-[5-(1-benzothien-3-yl)-1H-pyrrolo[2,3-b]pyridin-3-yl]-2-methylbenzoic acid;
dr) 6-{3-[4-(ethylsulfonyl)phenyl]-1H-pyrrolo[2,3-b]pyridin-5-yl}quinoline;
ds) 4-(5-(3-[(methylsulfonyl)amino]phenyl)-1H-pyrrolo[2,3-b]pyridin-3-yl)benzoic acid;
dt) 3-[4-(butyloxy)phenyl]-5-[3-(methylsulfonyl)phenyl]-1H-pyrrolo[2,3-b]pyridine;
du) N-(3-{3-[4-(aminomethyl)phenyl]-1H-pyrrolo[2,3-b]pyridin-5-yl}phenyl)methanesulfonamide;
dv) N-(3-{3-[3-(aminomethyl)phenyl]-1H-pyrrolo[2,3-b]pyridin-5-yl}phenyl)methanesulfonamide;
dw) 3-amino-5-(5-{3-[(methylsulfonyl)amino]phenyl}-1H-pyrrolo[2,3-b]pyridin-3-yl)benzoic acid;
dx) 2-fluoro-4-(5-{3-[(methylsulfonyl)amino]phenyl}-1H-pyrrolo[2,3-b]pyridin-3-yl)benzoic acid;
dy) 3-amino-5-{5-[3-(methylsulfonyl)phenyl]-1H-pyrrolo[2,3-b]pyridin-3-yl}benzoic acid;
dz) 2-fluoro-4-{5-[3-(methylsulfonyl)phenyl]-1H-pyrrolo[2,3-b]pyridin-3-yl}benzoic acid;
ea) [(4-{5-[3-(methylsulfonyl)phenyl]-1H-pyrrolo[2,3-b]pyridin-3-yl}phenyl)methyl]amine;
eb) [(3-{5-[3-(methylsulfonyl)phenyl]-1H-pyrrolo[2,3-b]pyridin-3-yl}phenyl)methyl]amine;
ec) 5-{5-[3-(methylsulfonyl)phenyl]-1H-pyrrolo[2,3-b]pyridin-3-yl}-2-thiophenecarbaldehyde;
ed) 4-{5-[3-(methylsulfonyl)phenyl]-1H-pyrrolo[2,3-b]pyridin-3-yl}benzoic acid;
ee) N-(4-{3-[4-(butyloxy)phenyl]-1H-pyrrolo[2,3-b]pyridin-5-yl}phenyl)methanesulfonamide;
ef) 1,1-dimethylethyl [2-(3-{5-[3-(methylsulfonyl)phenyl]-1H-pyrrolo[2,3-b]pyridin-3-yl}phenyl)ethyl]carbamate;
eg) 3-amino-5-(5-{4-[(methylsulfonyl)amino]phenyl}-1H-pyrrolo[2,3-b]pyridin-3-yl)benzoic acid;
eh) 2-fluoro-4-(5-{4-[(methylsulfonyl)amino]phenyl}-1H-pyrrolo[2,3-b]pyridin-3-yl)benzoic acid;
ei) 4-(5-{4-[(methylsulfonyl)amino]phenyl}-1H-pyrrolo[2,3-b]pyridin-3-yl)benzoic acid;
ej) N-(4-{3-[4-(aminomethyl)phenyl]-1H-pyrrolo[2,3-b]pyridin-5-yl}phenyl)methanesulfonamide;
ek) N-(4-{3-[3-(aminomethyl)phenyl]-1H-pyrrolo[2,3-b]pyridin-5-yl}phenyl)methanesulfonamide;
el) N-{4-[3-(5-formyl-2-thienyl)-1H-pyrrolo[2,3-b]pyridin-5-yl]phenyl}methanesulfonamide;
em) 7-{5-[3-(methylsulfonyl)phenyl]-1H-pyrrolo[2,3-b]pyridin-3-yl}-1,2,3,4-tetrahydroisoquinoline;
en) N-{3-[3-(1,2,3,4-tetrahydro-7-isoquinolinyl)-1H-pyrrolo[2,3-b]pyridin-5-yl]phenyl}methanesulfonamide;
eo) N-{4-[3-(1,2,3,4-tetrahydro-7-isoquinolinyl)-1H-pyrrolo[2,3-b]pyridin-5-yl]phenyl}methanesulfonamide;
ep) 2-methyl-4-{5-[3-(methylsulfonyl)phenyl]-1H-pyrrolo[2,3-b]pyridin-3-yl}benzoic acid;
eq) 5-{5-[3-(methylsulfonyl)phenyl]-1H-pyrrolo[2,3-b]pyridin-3-yl}-2-thiophenecarboxylic acid;
er) 3-{3-[3-(aminomethyl)phenyl]-1H-pyrrolo[2,3-b]pyridin-5-yl}benzonitrile;
es) 2-fluoro-4-{5-[6-(methyloxy)-3-pyridinyl]-1H-pyrrolo[2,3-b]pyridin-3-yl}benzoic acid;
et) 3-[3-(1,2,3,4-tetrahydro-7-isoquinolinyl)-1H-pyrrolo[2,3-b]pyridin-5-yl]benzonitrile;
eu) 7-{5-[3,4,5-tris(methyloxy)phenyl]-1H-pyrrolo[2,3-b]pyridin-3-yl}-1,2,3,4-tetrahydroisoquinoline;
ev) 4-{5-[3,4,5-tris(methyloxy)phenyl]-1H-pyrrolo[2,3-b]pyridin-3-yl}benzoic acid;
ew) 2-fluoro-4-{5-[3,4,5-tris(methyloxy)phenyl]-1H-pyrrolo[2,3-b]pyridin-3-yl}benzoic acid;
ex) 2-amino-4-{5-[3,4,5-tris(methyloxy)phenyl]-1H-pyrrolo[2,3-b]pyridin-3-yl}benzoic acid;
ey) 4-[5-(6-quinolinyl)-1H-pyrrolo[2,3-b]pyridin-3-yl]benzoic acid;
ez) 4-[5-(3-cyanophenyl)-1H-pyrrolo[2,3-b]pyridin-3-yl]-2-fluorobenzoic acid;
fa) 4-[5-(2,3-dimethylphenyl)-1H-pyrrolo[2,3-b]pyridin-3-yl]benzoic acid;
fb) 4-[5-(2,3-dimethylphenyl)-1H-pyrrolo[2,3-b]pyridin-3-yl]-2-fluorobenzoic acid;
fc) 2-methyl-4-[5-(6-quinolinyl)-1H-pyrrolo[2,3-b]pyridin-3-yl]benzoic acid;
fd) 2-methyl-4-[5-(1-naphthalenyl)-1H-pyrrolo[2,3-b]pyridin-3-yl]benzoic acid;
fe) 4-[5-(3-cyanophenyl)-1H-pyrrolo[2,3-b]pyridin-3-yl]-2-methylbenzoic acid;
ff) 2-methyl-4-{5-[6-(methyloxy)-3-pyridinyl]-1H-pyrrolo[2,3-b]pyridin-3-yl}benzoic acid;
fg) 2-methyl-4-{5-[3,4,5-tris(methyloxy)phenyl]-1H-pyrrolo[2,3-b]pyridin-3-yl}benzoic acid;
fh) 2-(1-methylethyl)-4-[5-(1-naphthalenyl)-1H-pyrrolo[2,3-b]pyridin-3-yl]benzoic acid;
fi) 4-[5-(3-cyanophenyl)-1H-pyrrolo[2,3-b]pyridin-3-yl]-2-(1-methylethyl)benzoic acid;
fj) 2-(1-methylethyl)-4-[5-(6-quinolinyl)-1H-pyrrolo[2,3-b]pyridin-3-yl]benzoic acid;
fk) 4-[5-(2,3-dimethylphenyl)-1H-pyrrolo[2,3-b]pyridin-3-yl]-2-methylbenzoic acid;
fl) 2-chloro-4-{5-[3-(methylsulfonyl)phenyl]-1-H-pyrrolo-[2,3-b]pyridin-3-yl}benzoic acid;
fm) 4-{5-[3-(methylsulfonyl)phenyl]-1-H-pyrrolo-[2,3-b]pyridin-3-yl}-2,6-bis(trifluoromethyl)benzoic acid;
fn) methyl 2-(azidomethyl)-4-(5-phenyl-1H-pyrrolo[2,3-b]pyridin-3-yl)benzoate;
fo) 2-ethyl-4-(5-phenyl-1H-pyrrolo[2,3-b]pyridin-3-yl)benzoic acid;
fp) 2-(methylamino)-4-(5-phenyl-1H-pyrrolo[2,3-b]pyridin-3-yl)benzoic acid;
fq) 2-(dimethylamino)-4-(5-phenyl-1H-pyrrolo[2,3-b]pyridin-3-yl)benzoic acid;
fr) 2-cyclopentyl-4-(5-phenyl-1H-pyrrolo[2,3-b]pyridin-3-yl)benzoic acid;
fs) 4-(5-phenyl-1H-pyrrolo[2,3-b]pyridin-3-yl)-2-propylbenzoic acid;
ft) 2,6-difluoro-4-(5-phenyl-1H-pyrrolo[2,3-b]pyridin-3-yl)benzoic acid;
fu) 2,6-dimethyl-4-(5-phenyl-1H-pyrrolo[2,3-b]pyridin-3-yl)benzoic acid;
fv) 2-(2-propyl)-4-(5-phenyl-1H-pyrrolo[2,3-b]pyridin-3-yl)benzoic acid;
fw) 6-(5-phenyl-1H-pyrrolo[2,3-b]pyridin-3-yl)-1H-indazole;
fx) 2-(2-methylpropyl)-4-(5-phenyl-1H-pyrrolo[2,3-b]pyridin-3-yl)benzoic acid;
fy) 2-methyl-4-(5-phenyl-1H-pyrrolo[2,3-b]pyridin-3-yl)benzoic acid;
fz) 4-[5-(3-hydroxyphenyl)-1-H-pyrrolo[2,3-b]pyridin-3-yl]benzoic acid;
ga) 3-amino-5-[5-(3-hydroxyphenyl)1H-pyrrolo[2,3-b]-pyridin-3-yl]-benzoic acid;
gb) {4-[5-hydroxyphenyl)-1H-pyrrolo[2,3-b]pyridin-3-yl]-phenyl}acetic acid;
gc) 4-[5-(3-aminophenyl)-1H-pyrrolo[2,3-b]pyridin-3-yl]benzoic acid;
gd) 3-{4-[5-(3-hydroxyphenyl)-1-H-pyrrolo[2,3-b]pyridine-3-yl]-phenyl}-propionic acid;
gd) 3-{4-[5-(3-aminophenyl)-1H-pyrrolo[2,3-b]pyridin-3-yl]phenyl}propanoic acid;
gf) {4-[5-(3-aminophenyl)-1H-pyrrolo[2,3-b]pyridin-3-yl]phenyl}acetic acid;
gg) 4-{5-[3-(aminomethyl)phenyl]-1H-pyrrolo[2,3-b]pyridin-3-yl}benzoic acid;
gh) (4-{5-[3-(aminomethyl)phenyl]-1H-pyrrolo[2,3-b]pyridin-3-yl}phenyl)acetic acid;
gi) 4-[5-(3-hydroxyphenyl)-1-H-pyrrolo-[2,3-b]pyridin-3-yl]benzoic acid;
gj) 2-fluoro-4-[5-(3-hydroxyphenyl)-1-H-pyrrolo-[2,3-b]pyridin-3-yl]benzoic acid;
gk) 5-[5-(3-hydroxyphenyl)-1-H-pyrrolo-[2,3-b]pyridin-3-yl]thiophene-2-carbaldehyde;
gl) 3-{3-[3-(2-aminoethyl)phenyl]-1-H-pyrrolo-[2,3-b]pyridin-5-yl}phenol;
gm) 3-[3-(1,2,3,4-tetrahydroisoquinolin-7-yl)-1-H-pyrrolo-[2,3-b]pyridin-5-yl]phenol;
gn) 3-amino-5-[5-(3-aminophenyl)-1-H-pyrrolo-[2,3-b]pyridin-3-yl]benzoic acid;
go) 4-[5-(3-aminophenyl)-1-H-pyrrolo-[2,3-b]pyridin-3-yl]-2-fluorobenzoic acid;
gp) 2-fluoro-4-(5-phenyl-1H-pyrrolo[2,3-b]pyridin-3-yl)phenol;
gq) 2,6-difluoro-4-(5-phenyl-1H-pyrrolo[2,3-b]pyridin-3-yl)phenol;
gr) 5-phenyl-3-[4-(1H-tetrazol-5-yl)phenyl]-1H-pyrrolo[2,3-b]pyridine;
gs) 5-(2-naphthalenyl)-3-[4-(1H-tetrazol-5-yl)phenyl]-1H-pyrrolo[2,3-b]pyridine;
gt) 3-[3-fluoro-4-(1H-tetrazol-5-yl)phenyl]-5-phenyl-1H-pyrrolo[2,3-b]pyridine;
gu) 2-methyl-2-(4-{5-[3-(methylsulfonyl)phenyl]-1H-pyrrolo[2,3-b]pyridin-3-yl}phenyl)propanoic acid;
gv) 2-{4-[5-(2-naphthalenyl)-1H-pyrrolo[2,3-b]pyridin-3-yl]phenyl}propanoic acid;
gw) 2-(4-{5-[3-(aminocarbonyl)phenyl]-1H-pyrrolo[2,3-b]pyridin-3-yl}phenyl)-2-methylpropanoic acid;
gx) 2-{4-[5-(3-cyanophenyl)-1H-pyrrolo[2,3-b]pyridin-3-yl]phenyl}-2-methylpropanoic acid;
gy) 2-methyl-2-{4-[5-(6-quinolinyl)-1H-pyrrolo[2,3-b]pyridin-3-yl]phenyl}propanoic acid;
gz) 2-methyl-2-{4-[5-(2-naphthalenyl)-1H-pyrrolo[2,3-b]pyridin-3-yl]phenyl}propanoic acid;
ha) 2-methyl-2-[4-(5-{3-[(methylsulfonyl)amino]phenyl}-1H-pyrrolo[2,3-b]pyridin-3-yl)phenyl]propanoic acid;
hb) 2-methyl-2-[4-(5-{4-[(methylsulfonyl)amino]phenyl}-1H-pyrrolo[2,3-b]pyridin-3-yl)phenyl]propanoic acid;
hc) 2-methyl-2-(4-{5-[6-(methyloxy)-2-naphthalenyl]-1H-pyrrolo[2,3-b]pyridin-3-yl}phenyl)propanoic acid;
hd) 2-methyl-2-{4-[5-(5-quinolinyl)-1H-pyrrolo[2,3-b]pyridin-3-yl]phenyl}propanoic acid;
he) 2-methyl-2-{4-[5-(3-quinolinyl)-1H-pyrrolo[2,3-b]pyridin-3-yl]phenyl}propanoic acid;
hf) 2-{4-[5-(6-quinolinyl)-1H-pyrrolo[2,3-b]pyridin-3-yl]phenyl}propanoic acid;
hg) 2-{4-[5-(5-quinolinyl)-1H-pyrrolo[2,3-b]pyridin-3-yl]phenyl}propanoic acid;
hh) 2-{4-[5-(3-quinolinyl)-1H-pyrrolo[2,3-b]pyridin-3-yl]phenyl}propanoic acid;
hi) 2-(4-{5-[3-(methylsulfonyl)phenyl]-1H-pyrrolo[2,3-b]pyridin-3-yl}phenyl)propanoic acid;
hj) 2-(4-{5-[6-(methyloxy)-2-naphthalenyl]-1H-pyrrolo[2,3-b]pyridin-3-yl}phenyl)propanoic acid;
hk) 2-methyl-2-[4-(5-phenyl-1H-pyrrolo[2,3-b]pyridin-3-yl)phenyl]propanoic acid;
hi) 2-methyl-2-{4-[5-(2-naphthalenyl)-1H-pyrrolo[2,3-b]pyridin-3-yl]phenyl}propanoic acid;
hm) 4-[5-(6-amino-3-pyridinyl)-1H-pyrrolo[2,3-b]pyridin-3-yl]benzoic acid;
hn) 4-{5-[6-(β-alanylamino)-3-pyridinyl]-1H-pyrrolo[2,3-b]pyridin-3-yl}benzoic acid;
ho) 4-(5-(6-indolyl)-1H-pyrrolo[2,3-b]pyridin-3-yl)benzoic acid;
hp) [2-(3-{5-[3-(methylsulfonyl)phenyl]-1H-pyrrolo[2,3-b]pyridin-3-yl}phenyl)ethyl]amine;
hq) N-(3-{3-[3-(2-aminoethyl)phenyl]-1H-pyrrolo[2,3-b]pyridin-5-yl}phenyl)methanesulfonamide;
hr) N-(4-{3-[3-(2-aminoethyl)phenyl]-1H-pyrrolo[2,3-b]pyridin-5-yl}phenyl)methanesulfonamide;
hs) 4-{5-[3-(2-aminoethyl)phenyl]-1-H-pyrrolo-[2,3-b]pyridin-3-yl}benzoic acid;
ht) 4-{5-[3-(2-aminoethyl)phenyl]-1-H-pyrrolo-[2,3-b]pyridin-3-yl}-2-methylbenzoic acid;
hu) 4-{5-[3-({[2-(dimethylamino)ethyl]amino)carbonyl)phenyl]-1H-pyrrolo[2,3-b]pyridin-3-yl}benzoic acid;
hv) 4-(5-(3-[4-(1,1-dimethylethyloxycarbonyl)aminobutanoyl]amino)phenyl-1H-pyrrolo[2,3-b]pyridin-3-yl)benzoic acid;
hw) 4-(5-(3-[3-(1,1-dimethylethyloxycarbonyl)aminopropanoyl]amino)phenyl-1H-pyrrolo[2,3-b]pyridin-3-yl)benzoic acid;
hx) 2-(2-propyl)-4-[5-(3-[3-(1,1-dimethylethyloxycarbonyl)aminopropanoyl]amino)phenyl-1H-pyrrolo[2,3-b]pyridin-3-yl]benzoic acid;
hy) 4-{5-[3-(β-alanylamino)phenyl]-1H-pyrrolo[2,3-b]pyridin-3-yl}benzoic acid;
hz) 4-(5-{3-[(4-aminobutanoyl)amino]phenyl}-1H-pyrrolo[2,3-b]pyridin-3-yl)benzoic acid;
ia) 4-{5-[3-(beta-alanylamino)phenyl]-1H-pyrrolo[2,3-b]pyridin-3-yl}-2-methylbenzoic acid;
ib) 4-{5-[3-(beta-alanylamino)phenyl]-1H-pyrrolo[2,3-b]pyridin-3-yl}-2-(1-methylethyl)benzoic acid;
ic) 4-[5-(3-{[(2-aminoethyl)amino]carbonyl}phenyl)-1H-pyrrolo[2,3-b]pyridin-3-yl]benzoic acid;
id) 4-[5-(3-{[(3-aminopropyl)amino]carbonyl}phenyl)-1H-pyrrolo[2,3-b]pyridin-3-yl]benzoic acid;
ie) 3-amino-5-[5-(3-aminophenyl)-1-H-pyrrolo-[2,3-b]pyridin-3-yl]benzoic acid;
if) 3-amino-5-{5-[3-(aminomethyl)phenyl]-1-H-pyrrolo-[2,3-b]pyridin-3-yl}benzoic acid;
ig) 4-{5-[3-(aminomethyl)phenyl]-1-H-pyrrolo-[2,3-b]pyridin-3-yl}-2-fluorobenzoic acid;
ih) 2-(aminomethyl)-4-(5-phenyl-1H-pyrrolo[2,3-b]pyridin-3-yl)benzoic acid;
ii) 3-{3-[3-(2-aminoethyl)phenyl]-1H-pyrrolo[2,3-b]pyridin-5-yl}benzonitrile; and
ij) 4-[5-(3-amino-1-isoquinolinyl)-1H-pyrrolo[2,3-b]pyridin-3-yl]-2-fluorobenzoic acid;
or a pharmaceutically acceptable salt or solvate thereof.

15. A pharmaceutical composition comprising a therapeutically effective amount of a compound as recited in claim 6 or 14, or a pharmaceutically acceptable salt or solvate thereof, and one or more of pharmaceutically acceptable carriers, diluents and excipients.
